(19) United States
(12) Patent Application Publication
(10) Pub. No.: US 2026/0207590 A1
(43) Pub. Date: Jul. 23, 2026

(54) CTPS1 INHIBITORS FOR USE IN THE TREATMENT OF CTPS2 DEFICIENT CANCER

(71) Applicant: STEP PHARMA S.A.S., Paris, Cedex 06 (FR)

(72) Inventors: Philip BEER, Paris (FR); Andrew PARKER, Paris (FR)

(21) Appl. No.: 19/140,541

(22) PCT Filed: Dec. 21, 2023
(86) PCT No.: PCT/EP2023/087338

§ 371 Date: Jun. 18, 2025

(30) Foreign Application Priority Data
Dec. 21, 2022 (EP) 22306966.7

(51) Int. Cl. A61K 31/506

(52) U.S. Cl. CPC A61K 31/506

(57) ABSTRACT

The invention provides, inter alia, methods of treating CTPS2 deficient cancers by administering CTPS1 inhibitors, and related aspects.

### FIELD OF THE INVENTION

The invention relates to methods of treating CTPS2 deficient cancers by administering CTPS1 inhibitors, and related aspects.

### BACKGROUND OF THE INVENTION

Cancer can affect multiple cell types and tissues but the underlying cause is a breakdown in the control of cell division. This process is highly complex, requiring careful coordination of multiple pathways, many of which remain to be fully characterised. Cell division requires the effective replication of the cell's DNA and other constituents. Interfering with a cell's ability to replicate by targeting nucleic acid synthesis has been a core approach in cancer therapy for many years. Examples of therapies acting in this way are 6-thioguanine, 6-mecaptopurine, 5-fluorouracil, cytarabine, gemcitabine and pemetrexed.

Cancer therapeutics against a wide array of specific targets are available. Small molecule targeted therapy drugs are generally inhibitors of enzymatic domains on mutated, overexpressed, or otherwise critical proteins within the cancer cell. Monoclonal antibody therapy is another strategy in which the therapeutic agent is an antibody which specifically binds to a protein on the surface of the cancer cells.

All proliferating cells, including neoplastic cells, are reliant on a ready source of purine and pyrimidine nucleotides for DNA and RNA synthesis. Whilst salvage pathways may be sufficient for steady state metabolism, DNA replication to enable cell division is dependent on synthesis of nucleotides via the de novo pathway. A key bottleneck in the de novo pyrimidine synthesis pathway is the enzyme cytidine triphosphate synthase (CTPS) which catalyses the conversion of UTP to CTP (van Kuilenburg 2000). CTPS also has two isoforms in humans (CTPS1 and CTPS2; see FIG. 1). Both isoforms are ubiquitously expressed in normal and malignant human cells (BioGPS and EMBL-EBI Expression Atlas). Differential roles of CTPS1 and CTPS2 have been shown in cell proliferation (Minet 2023), with human genetic studies have identified an essential and non-redundant role for CTPS1 in the proliferation of normal immune (B and T) cells (Martin 2014; Martin 2020).

Whilst cancer cells are dependent on CTPS activity in order to proliferate, the precise role that CTPS1 and CTPS2 play in cancer is currently not completely clear. Several CTPS inhibitors that inhibit both CTPS1 and CTPS2 have been developed for oncology indications up to phase I/II clinical trials, but were stopped due to toxicity and efficacy issues. Most of the developed inhibitors are nucleoside-analogue prodrugs (3-deazauridine (DAU), CPEC, carbodine), which are converted to the active triphosphorylated metabolite by the kinases involved in pyrimidine biosynthesis: uridine/cytidine kinase, nucleoside monophosphate-kinase (NMP-kinase) and nucleoside diphosphatekinase (NDP-kinase). Other inhibitors (acivicin, 6-diazo-5-oxo-L-norleucine (DON)) are reactive analogues of glutamine, which irreversibly inhibit the glutaminase domain of CTPS. Importantly, none of the inhibitors of CTPS developed to date are selective for one isoform of CTPS over the other. As such, available CTPS inhibitors block all CTPS activity and, therefore, block the ability of all cells in the body to undergo cell division.

Cancer is a disease of the genome. Cancer cells harbour different types of alterations in their DNA that underpin the biological changes in cell biology that define cancer. These DNA alterations include base level mutations as well as gene and chromosome level structural alterations such as deletions, amplifications and gene fusions. Cancer cells harbour widespread genomic disruption; however, only a small proportion of these changes directly alter cell biology, with the remainder constituting collateral genomic damage or bystander effects from the mutational processes that result in the genetic changes. For example, deletion of genetic material in the cancer cell may result in loss of a specific tumour suppressor gene that increases the fitness of the cancer cell (and thus contributes to the process of cancer development); this same deletion may also result in loss of other genes in the same region which are of no consequence to the cancer cell. In some such cases, deletion of genes with no consequence to the cancer cell may generate a state where the cancer cells become critically dependent on a specific metabolic pathway or enzyme, a situation that may be termed metabolic collateral lethality. As an example, deletion of CTPS2 from a cancer cell does not have any direct effect on the fitness of the cell (FIG. 2), but creates a critical dependence on the activity of CTPS1 in order to generate the building blocks required for DNA synthesis (and thus cell division). In humans, CTPS2 is found on the X chromosome (ChrX (p22.2); transcript IDs ENST00000359276.9, NM_175859; Position: hg38 chrX:16,587,999-16,712,669).

There remains a need for new approaches to cancer therapies, such approaches may demonstrate high in vivo efficacy, reduction in the dose required for effect in vivo, an improved safety profile/reduced side effects, or the like.

### SUMMARY OF THE INVENTION

The invention provides a method for the treatment of CTPS2 deficient cancer in a subject, the method comprising administering a CTPS1 inhibitor to the subject.

The invention also provides a method for the treatment of cancer in a subject, the method comprising the steps of:
i) identifying that the subject has a cancer which is deficient in CTPS2; and
ii) administering a CTPS1 inhibitor to the subject.

The invention also provides a method for the treatment of cancer in a subject, the method comprising the steps of:
i) providing a sample from the subject;
ii) identifying using the sample that a subject has a cancer which is deficient in CTPS2; and
iii) administering a CTPS1 inhibitor to the subject.

The invention also provides a method for the treatment of cancer in a subject, the method comprising the steps of:
i) obtaining a sample from the subject;
ii) identifying using the sample that a subject has a cancer which is deficient in CTPS2; and
iii) administering a CTPS1 inhibitor to the subject.

The invention also provides a method for the treatment of cancer which may be susceptible to treatment with a CTPS1 inhibitor in a subject, the method comprising the steps of:
i) identifying that the cancer may be deficient in CTPS2; and
ii) administering a CTPS1 inhibitor to the subject.

The invention also provides a method for the treatment of cancer which may be susceptible to treatment with a CTPS1 inhibitor in a subject, the method comprising the steps of:
i) providing a sample from the subject;
ii) identifying using the sample that the cancer may be deficient in CTPS2; and
iii) administering a CTPS1 inhibitor.

The invention also provides a method for the treatment of cancer which may be susceptible to treatment with a CTPS1 inhibitor in a subject, the method comprising the steps of:
i) obtaining a sample from the subject;
ii) identifying using the sample that the cancer may be deficient in CTPS2; and
iii) administering a CTPS1 inhibitor.

The invention also provides a method of determining that cancer cells may be susceptible to treatment with a CTPS1 inhibitor, the method comprising the step of identifying that the cancer cells are deficient in CTPS2.

The invention also provides a method of determining that a cancer in a subject may be susceptible to treatment with a CTPS1 inhibitor, the method comprising the step of identifying that the cancer is deficient in CTPS2.

The invention also provides a method of determining that cancer cells may be susceptible to treatment with a CTPS1 inhibitor, the method comprising the step of identifying that the cancer cells may be deficient in CTPS2.

The invention also provides a method of determining that a cancer in a subject may be susceptible to treatment with a CTPS1 inhibitor, the method comprising the step of identifying that the cancer may be deficient in CTPS2.

The invention also provides a CTPS1 inhibitor for use in the treatment of CTPS2 deficient cancer. The invention also provides a CTPS1 inhibitor for use in the treatment of CTPS2 deficient cancer in a subject.

The invention also provides a CTPS1 inhibitor for use in the treatment of cancer which may be deficient in CTPS2. The invention also provides a CTPS1 inhibitor for use in the treatment of cancer which may be deficient in CTPS2 in a subject.

The invention also provides the use of a CTPS1 inhibitor in the manufacture of a medicament for the treatment of CTPS2 deficient cancer. The invention also provides the use of a CTPS1 inhibitor in the manufacture of a medicament for the treatment of CTPS2 deficient cancer in a subject.

The invention also provides the use of a CTPS1 inhibitor in the manufacture of a medicament for the treatment of cancer which may be deficient in CTPS2. The invention also provides the use of a CTPS1 inhibitor in the manufacture of a medicament for the treatment of cancer which may be deficient in CTPS2 in a subject.

The invention also provides a pharmaceutical composition comprising a CTPS1 inhibitor and a pharmaceutically acceptable excipient or carrier for use in the treatment of CTPS2 deficient cancer. The invention also provides a pharmaceutical composition comprising a CTPS1 inhibitor and a pharmaceutically acceptable excipient or carrier for use in the treatment of CTPS2 deficient cancer in a subject.

The invention also provides a pharmaceutical composition comprising a CTPS1 inhibitor and a pharmaceutically acceptable excipient or carrier for use in the treatment of cancer which may be deficient in CTPS2. The invention also provides a pharmaceutical composition comprising a CTPS1 inhibitor and a pharmaceutically acceptable excipient or carrier for use in the treatment of cancer which may be deficient in CTPS2 in a subject.

### Summary of the Sequences

SEQ ID NO: 1
FLAG-His₈-tag
SEQ ID NO: 2
FLAG-His-Avi tag

### SUMMARY OF THE FIGURES

FIG. 1: De novo CTP production pathway.

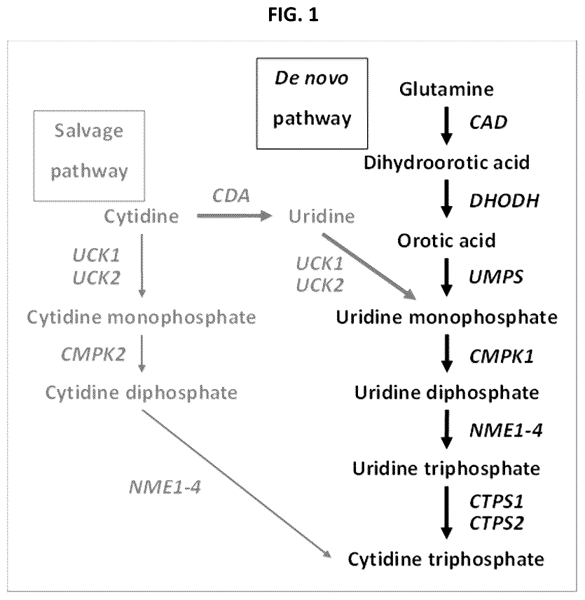

FIG. 2: Achilles CRISPR screen comprising 1032 cancer cell lines; graph shows box and whisker plots representing number of cell lines by median, interquartile range and overall range; x-axis shows CERES score: 0=no effect, <0=reduced proliferation, -1=median of all common essential genes.

FIG. 3: Analysis of whole genome sequencing data from 2,348 cancer samples in the ICGC PCAWG cohort, accessed via cBioPortal, showing proportion of samples by cancer type with a homozygous deletion of CTPS2. NSCLC, non-small cell lung cancer; CNS, central nervous system.

FIG. 4: Analysis of 573 cancer cell lines with CTPS1 knockout data from the Achilles project and CTPS2 RNA expression from the Cancer Cell Line Encyclopedia; data accessed via the CellMinerCDB portal.

FIG. 5: Comparison of CTPS2 loss ascertained by whole genome sequencing data from samples in the ICGC PCAWG cohort with CTPS2 loss ascertained by immunohistochemistry analysis of tumour microarrays.

### DETAILED DESCRIPTION OF THE INVENTION

The inventors have identified that some cancers are deficient in CTPS2, and therefore are likely to be particularly receptive to treatment with a CTPS1 inhibitor.

The invention provides a method for the treatment of CTPS2 deficient cancer in a subject, the method comprising administering a CTPS1 inhibitor to the subject.

The invention also provides a method for the treatment of cancer in a subject, the method comprising the steps of:
i) identifying that the subject has a cancer which is deficient in CTPS2; and
ii) administering a CTPS1 inhibitor to the subject.

The invention also provides a method for the treatment of cancer in a subject, the method comprising the steps of:
i) providing a sample from the subject;
ii) identifying using the sample that a subject has a cancer which is deficient in CTPS2; and
iii) administering a CTPS1 inhibitor to the subject.

The invention also provides a method for the treatment of cancer in a subject, the method comprising the steps of:
i) obtaining a sample from the subject;
ii) identifying using the sample that a subject has a cancer which is deficient in CTPS2; and
iii) administering a CTPS1 inhibitor to the subject.

The invention also provides a method for the treatment of cancer which may be susceptible to treatment with a CTPS1 inhibitor in a subject, the method comprising the steps of:
i) identifying that the cancer may be deficient in CTPS2; and
ii) administering a CTPS1 inhibitor to the subject.

The invention also provides a method for the treatment of cancer which may be susceptible to treatment with a CTPS1 inhibitor in a subject, the method comprising the steps of:
i) providing a sample from the subject;
ii) identifying using the sample that the cancer may be deficient in CTPS2; and
iii) administering a CTPS1 inhibitor.

The invention also provides a method for the treatment of cancer which may be susceptible to treatment with a CTPS1 inhibitor in a subject, the method comprising the steps of:
i) obtaining a sample from the subject;
ii) identifying using the sample that the cancer may be deficient in CTPS2; and
iii) administering a CTPS1 inhibitor.

The invention also provides a method of determining that cancer cells may be susceptible to treatment with a CTPS1 inhibitor, the method comprising the step of identifying that the cancer cells are deficient in CTPS2.

The invention also provides a method of determining that a cancer in a subject may be susceptible to treatment with a CTPS1 inhibitor, the method comprising the step of identifying that the cancer is deficient in CTPS2.

The invention also provides a method of determining that cancer cells may be susceptible to treatment with a CTPS1 inhibitor, the method comprising the step of identifying that the cancer cells may be deficient in CTPS2.

The invention also provides a method of determining that a cancer in a subject may be susceptible to treatment with a CTPS1 inhibitor, the method comprising the step of identifying that the cancer may be deficient in CTPS2.

The invention also provides a CTPS1 inhibitor for use in the treatment of CTPS2 deficient cancer. The invention also provides a CTPS1 inhibitor for use in the treatment of CTPS2 deficient cancer in a subject.

The invention also provides a CTPS1 inhibitor for use in the treatment of cancer which may be deficient in CTPS2. The invention also provides a CTPS1 inhibitor for use in the treatment of cancer which may be deficient in CTPS2 in a subject.

The invention also provides the use of a CTPS1 inhibitor in the manufacture of a medicament for the treatment of CTPS2 deficient cancer. The invention also provides the use of a CTPS1 inhibitor in the manufacture of a medicament for the treatment of CTPS2 deficient cancer in a subject.

The invention also provides the use of a CTPS1 inhibitor in the manufacture of a medicament for the treatment of cancer which may be deficient in CTPS2. The invention also provides the use of a CTPS1 inhibitor in the manufacture of a medicament for the treatment of cancer which may be deficient in CTPS2 in a subject.

The invention also provides a pharmaceutical composition comprising a CTPS1 inhibitor and a pharmaceutically acceptable excipient or carrier for use in the treatment of CTPS2 deficient cancer. The invention also provides a pharmaceutical composition comprising a CTPS1 inhibitor and a pharmaceutically acceptable excipient or carrier for use in the treatment of CTPS2 deficient cancer in a subject.

The invention also provides a pharmaceutical composition comprising a CTPS1 inhibitor and a pharmaceutically acceptable excipient or carrier for use in the treatment of cancer which may be deficient in CTPS2. The invention also provides a pharmaceutical composition comprising a CTPS1 inhibitor and a pharmaceutically acceptable excipient or carrier for use in the treatment of cancer which may be deficient in CTPS2 in a subject.

CTPS1 inhibitors A CTPS1 inhibitor, as used herein, is an agent which directly inhibits the enzymatic activity of the CTPS1 enzyme through interaction with the enzyme. Direct inhibition of the CTPS1 enzyme may be quantified using any suitable assay procedure, though is suitably performed using the procedure set out in Example 1.

CTPS1 inhibitors may demonstrate an IC₅₀ of 10 uM or lower, such as 1 uM or lower, especially 100 nM or lower, in respect of CTPS1 enzyme. CTPS1 inhibitors of particular interest are those demonstrating an IC₅₀ of 10 uM or lower, such as 1 uM or lower, especially 100 nM or lower, in respect of CTPS1 enzyme using the assay procedure set out in Example 1.

CTPS1 inhibitors ideally demonstrate a selectivity for CTPS1 over CTPS2 (i.e. by ratio of IC₅₀ values). Suitably the inhibitors demonstrate a selectivity of at least 2-fold, such as at least 30-fold, especially at least 60-fold and in particular at least 1000-fold. CTPS1 inhibitors of particular interest are those demonstrating a selectivity for CTPS1 over CTPS2, suitably of at least 2-fold, such as at least 30-fold, especially at least 60-fold and in particular at least 1000-fold using the assay procedure set out in Example 2. Desirably the selectivity is for human CTPS1 over human CTPS2.

In the case of medicaments intended for human use, CTPS1 inhibition and CTPS1 vs CTPS2 selectivity should be based on human forms of the enzymes.

Suitably the CTPS1 inhibitor may be selected from the following compounds:

A compound of formula (I):

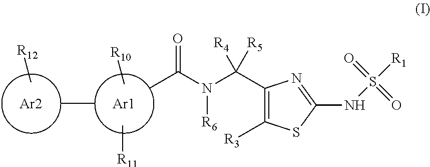

wherein
R₁ is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R₃ is H, CH₃, halo, OC₁₋₂alkyl or CF₃;
R₄ and R₅ are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl,
   or R₄ and R₅ together with the carbon atom to which they are attached form a C₃₋₆cycloalkyl or C₃₋₆heterocycloalkyl ring;
R₆ is H or C₁₋₃alkyl;
Ar1 is a 6-membered aryl or heteroaryl;
Ar2 is a 6-membered aryl or heteroaryl and is attached to Ar1 in the para position relative to the amide;
R₁₀ is H, halo, C₁₋₃alkyl, OC₁₋₂alkyl, C₁₋₂haloalkyl, OC₁₋₂haloalkyl or CN;
R₁₁ is H, F, Cl, CH₃, ethyl, OCH₃, CF₃, OCF₃ or CN;
R₁₂ is attached to Ar2 in the meta or ortho position relative to Ar1 and R₁₂ is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR₂₃R₂₄, SO₂CH₃, C(O)N(CH₃)₂, NHC(O)C₁₋₃alkyl, or a C₃₋₆heterocycloalkyl comprising one nitrogen located at the point of attachment to Ar2, or R₁₂ together with a nitrogen atom to which it is attached forms an N-oxide (N^{+—O—}
R₂₃ is H or C₁₋₂alkyl;
R₂₄ is H or C₁₋₂alkyl;
or a pharmaceutically acceptable salt and/or pharmaceutically acceptable solvate thereof.

More suitably the CTPS1 inhibitor is selected from the following (‘List A’) compounds:
N-((2-(cyclopropanesulfonamido)thiazol-4-yl)methyl)-5-phenylpicolinamide;
N-((2-(cyclopropanesulfonamido)thiazol-4-yl)methyl)-4-(pyridin-3-yl)benzamide;
N-(1-(2-(cyclopropanesulfonamido)thiazol-4-yl)propyl)-4-(5-(trifluoromethyl)pyridin-3-yl)benzamide;
N-(1-(2-(cyclopropanesulfonamido)thiazol-4-yl)propyl)-4-(5-(trifluoromethyl)pyridin-3-yl)benzamide (R enantiomer);
N-(1-(2-(cyclopropanesulfonamido)thiazol-4-yl)propyl)-4-(5-(trifluoromethyl)pyridin-3-yl)benzamide (S enantiomer);
N-(1-(2-(cyclopropanesulfonamido)thiazol-4-yl)propyl)-4-(6-(trifluoromethyl)pyrazin-2-yl)benzamide;
N-(2-(2-(cyclopropanesulfonamido)thiazol-4-yl)propan-2-yl)-4-(6-ethoxypyrazin-2-yl)-2-fluorobenzamide;
N-(2-(2-(cyclopropanesulfonamido)thiazol-4-yl)propan-2-yl)-4-(6-ethoxypyrazin-2-yl)-2-methoxybenzamide;
N-((2-(cyclopropanesulfonamido)thiazol-4-yl)methyl)-[1,1′-biphenyl]-4-carboxamide;
N-((2-(cyclopropanesulfonamido)thiazol-4-yl)methyl)-2-fluoro-4-(6-(trifluoromethyl)pyrazin-2-yl)benzamide;
N-((2-(cyclopropanesulfonamido)thiazol-4-yl)methyl)-4-(6-ethoxypyrazin-2-yl)-2-fluorobenzamide;
N-((2-(cyclopropanesulfonamido)thiazol-4-yl)methyl)-4-(6-(trifluoromethyl)pyrazin-2-yl)benzamide;
N-((2-(cyclopropanesulfonamido)thiazol-4-yl)methyl)-4-(6-isopropoxypyrazin-2-yl)benzamide;
N-((2-(cyclopropanesulfonamido)thiazol-4-yl)methyl)-4-(6-ethoxypyrazin-2-yl)benzamide;
N-(3-(2-(cyclopropanesulfonamido)thiazol-4-yl)pentan-3-yl)-4-(5-(trifluoromethyl)pyridin-3-yl)benzamide;
N-(3-(2-(cyclopropanesulfonamido)thiazol-4-yl)pentan-3-yl)-4-(5-fluoropyridin-3-yl)benzamide;
N-(3-(2-(cyclopropanesulfonamido)thiazol-4-yl)pentan-3-yl)-4-(5-methylpyridin-3-yl)benzamide;
N-(3-(2-(cyclopropanesulfonamido)thiazol-4-yl)pentan-3-yl)-4-(pyridin-3-yl)benzamide;
N-(3-(2-(cyclopropanesulfonamido)thiazol-4-yl)pentan-3-yl)-4-(6-(trifluoromethyl)pyrazin-2-yl)benzamide;
4-(6-chloropyrazin-2-yl)-N-(3-(2-(cyclopropanesulfonamido)thiazol-4-yl)pentan-3-yl)benzamide;
N-(3-(2-(cyclopropanesulfonamido)thiazol-4-yl)pentan-3-yl)-4-(6-methylpyrazin-2-yl)benzamide;
N-(3-(2-(cyclopropanesulfonamido)thiazol-4-yl)pentan-3-yl)-4-(pyrazin-2-yl)benzamide;
N-(2-(2-(cyclopropanesulfonamido)thiazol-4-yl)propan-2-yl)-5-(6-ethoxypyrazin-2-yl)-3-fluoropicolinamide;
N-(2-(2-(cyclopropanesulfonamido)thiazol-4-yl)propan-2-yl)-5-(6-(trifluoromethyl)pyrazin-2-yl)picolinamide;
5-(6-chloropyrazin-2-yl)-N-(2-(2-(cyclopropanesulfonamido)thiazol-4-yl)propan-2-yl)picolinamide;
N-(2-(2-(cyclopropanesulfonamido)thiazol-4-yl)propan-2-yl)-5-(6-ethoxypyrazin-2-yl)picolinamide;
N-(2-(2-(cyclopropanesulfonamido)thiazol-4-yl)propan-2-yl)-[2,2′-bipyridine]-5-carboxamide;
4-(5-chloropyridin-3-yl)-N-(2-(2-(cyclopropanesulfonamido)thiazol-4-yl)propan-2-yl)benzamide;
N-(2-(2-(cyclopropanesulfonamido)thiazol-4-yl)propan-2-yl)-2-fluoro-4-(5-(trifluoromethyl)pyridin-3-yl)benzamide;
4-(5-chloropyridin-3-yl)-N-(2-(2-(cyclopropanesulfonamido)thiazol-4-yl)propan-2-yl)-2-fluorobenzamide;
N-(2-(2-(cyclopropanesulfonamido)thiazol-4-yl)propan-2-yl)-2-fluoro-4-(5-fluoropyridin-3-yl)benzamide;
N-(2-(2-(cyclopropanesulfonamido)thiazol-4-yl)propan-2-yl)-2-methoxy-4-(5-(trifluoromethyl)pyridin-3-yl)benzamide;
4-(5-acetylpyridin-3-yl)-N-(2-(2-(cyclopropanesulfonamido)thiazol-4-yl)propan-2-yl)benzamide;
N-(2-(2-(cyclopropanesulfonamido)thiazol-4-yl)propan-2-yl)-4-(5-(trifluoromethyl)pyridin-3-yl)benzamide;
N-(2-(2-(cyclopropanesulfonamido)thiazol-4-yl)propan-2-yl)-4-(5-fluoropyridin-3-yl)benzamide;
N-(2-(2-(cyclopropanesulfonamido)thiazol-4-yl)propan-2-yl)-4-(5-methylpyridin-3-yl)benzamide;
N-(2-(2-(cyclopropanesulfonamido)thiazol-4-yl)propan-2-yl)-4-(5-methoxypyridin-3-yl)benzamide;
N-(2-(2-(cyclopropanesulfonamido)thiazol-4-yl)propan-2-yl)-4-(pyridin-3-yl)benzamide;
N-(2-(2-(cyclopropanesulfonamido)thiazol-4-yl)propan-2-yl)-3′-(trifluoromethyl)-[1,1′-biphenyl]-4-carboxamide;
N-(2-(2-(cyclopropanesulfonamido)thiazol-4-yl)propan-2-yl)-4-(6-ethylpyrazin-2-yl)-2-fluorobenzamide;
N-(2-(2-(cyclopropanesulfonamido)thiazol-4-yl)propan-2-yl)-2-fluoro-4-(6-(trifluoromethyl)pyrazin-2-yl)benzamide;
N-(2-(2-(cyclopropanesulfonamido)thiazol-4-yl)propan-2-yl)-2-fluoro-4-(6-isopropoxypyrazin-2-yl)benzamide;
N-(2-(2-(cyclopropanesulfonamido)thiazol-4-yl)propan-2-yl)-2-fluoro-4-(6-(2,2,2-trifluoroethoxy)pyrazin-2-yl)benzamide;
N-(2-(2-(cyclopropanesulfonamido)thiazol-4-yl)propan-2-yl)-2-methyl-4-(6-(trifluoromethyl)pyrazin-2-yl)benzamide;
N-(2-(2-(cyclopropanesulfonamido)thiazol-4-yl)propan-2-yl)-4-(6-ethoxypyrazin-2-yl)-2-methylbenzamide;
N-(2-(2-(cyclopropanesulfonamido)thiazol-4-yl)propan-2-yl)-4-(6-ethoxypyrazin-2-yl)-2-(trifluoromethyl)benzamide;
N-(2-(2-(cyclopropanesulfonamido)thiazol-4-yl)propan-2-yl)-2-methoxy-4-(6-(trifluoromethyl)pyrazin-2-yl)benzamide;
4-(6-chloropyrazin-2-yl)-N-(2-(2-(cyclopropanesulfonamido)thiazol-4-yl)propan-2-yl)-2-methoxybenzamide;
4-(6-cyanopyrazin-2-yl)-N-(2-(2-(cyclopropanesulfonamido)thiazol-4-yl)propan-2-yl)-2-methoxybenzamide;
N-(2-(2-(cyclopropanesulfonamido)thiazol-4-yl)propan-2-yl)-4-(6-(trifluoromethyl)pyrazin-2-yl)benzamide;
4-(6-chloropyrazin-2-yl)-N-(2-(2-(cyclopropanesulfonamido)thiazol-4-yl)propan-2-yl)benzamide;
N-(2-(2-(cyclopropanesulfonamido)thiazol-4-yl)propan-2-yl)-4-(6-methylpyrazin-2-yl)benzamide;
N-(2-(2-(cyclopropanesulfonamido)thiazol-4-yl)propan-2-yl)-4-(6-methoxypyrazin-2-yl)benzamide;
N-(2-(2-(cyclopropanesulfonamido)thiazol-4-yl)propan-2-yl)-4-(6-ethoxypyrazin-2-yl)benzamide;
N-(2-(2-(cyclopropanesulfonamido)thiazol-4-yl)propan-2-yl)-4-(6-isopropoxypyrazin-2-yl)benzamide;
N-(2-(2-(cyclopropanesulfonamido)thiazol-4-yl)propan-2-yl)-4-(6-(2,2,2-trifluoroethoxy)pyrazin-2-yl)benzamide;
N-(2-(2-(cyclopropanesulfonamido)thiazol-4-yl)propan-2-yl)-4-(pyrazin-2-yl)benzamide;
N-(1-(2-(cyclopropanesulfonamido)thiazol-4-yl)propyl)-4-(5-fluoropyridin-3-yl)benzamide;
N-(1-(2-(cyclopropanesulfonamido)thiazol-4-yl)propyl)-4-(5-methylpyridin-3-yl)benzamide;
N-(1-(2-(cyclopropanesulfonamido)thiazol-4-yl)propyl)-4-(pyridin-3-yl)benzamide;
N-(1-(2-(cyclopropanesulfonamido)thiazol-4-yl)propyl)-4-(6-ethoxypyrazin-2-yl)-2-fluorobenzamide;
N-(1-(2-(cyclopropanesulfonamido)thiazol-4-yl)propyl)-4-(6-ethoxypyrazin-2-yl)-2-fluoro-N-methylbenzamide;
N-(1-(2-(cyclopropanesulfonamido)thiazol-4-yl)propyl)-2-fluoro-4-(6-isopropoxypyrazin-2-yl)benzamide;
4-(6-chloropyrazin-2-yl)-N-(1-(2-(cyclopropanesulfonamido)thiazol-4-yl)propyl)benzamide;
N-(1-(2-(cyclopropanesulfonamido)thiazol-4-yl)propyl)-4-(6-methylpyrazin-2-yl)benzamide;
N-(1-(2-(cyclopropanesulfonamido)thiazol-4-yl)propyl)-4-(pyrazin-2-yl)benzamide;
N-(1-(2-(cyclopropanesulfonamido)thiazol-4-yl)propyl)-4-(5-fluoropyridin-3-yl)benzamide (R enantiomer);
N-(1-(2-(cyclopropanesulfonamido)thiazol-4-yl)propyl)-4-(5-fluoropyridin-3-yl)benzamide (S enantiomer);
N-(1-(2-(cyclopropanesulfonamido)thiazol-4-yl)propyl)-4-(6-ethoxypyrazin-2-yl)-2-fluorobenzamide (R enantiomer);
N-(1-(2-(cyclopropanesulfonamido)thiazol-4-yl)propyl)-4-(6-ethoxypyrazin-2-yl)-2-fluorobenzamide (S enantiomer);
N-(2-(2-(cyclopropanesulfonamido)-5-methylthiazol-4-yl)propan-2-yl)-5-(6-ethoxypyrazin-2-yl)picolinamide;
N-(2-(5-chloro-2-(cyclopropanesulfonamido)thiazol-4-yl)propan-2-yl)-5-(6-ethoxypyrazin-2-yl)picolinamide;
N-(2-(2-(cyclopropanesulfonamido)-5-methylthiazol-4-yl)propan-2-yl)-4-(6-ethoxypyrazin-2-yl)-2-fluorobenzamide;
N-(2-(5-chloro-2-(cyclopropanesulfonamido)thiazol-4-yl)propan-2-yl)-4-(6-ethoxypyrazin-2-yl)-2-fluorobenzamide;
N-(2-(2-(cyclopropanesulfonamido)-5-methylthiazol-4-yl)propan-2-yl)-2-methyl-4-(6-(trifluoromethyl)pyrazin-2-yl)benzamide;
N-(2-(5-chloro-2-(cyclopropanesulfonamido)thiazol-4-yl)propan-2-yl)-2-methyl-4-(6-(trifluoromethyl)pyrazin-2-yl)benzamide;
N-(2-(2-(cyclopropanesulfonamido)-5-methylthiazol-4-yl)propan-2-yl)-4-(6-(trifluoromethyl)pyrazin-2-yl)benzamide;
N-(2-(5-chloro-2-(cyclopropanesulfonamido)thiazol-4-yl)propan-2-yl)-4-(6-(trifluoromethyl)pyrazin-2-yl)benzamide;
N-(1-(2-(cyclopropanesulfonamido)thiazol-4-yl)cyclopropyl)-5-(6-ethoxypyrazin-2-yl)picolinamide;
N-(1-(2-(cyclopropanesulfonamido)thiazol-4-yl)cyclopropyl)-4-(pyridin-3-yl)benzamide;
N-(1-(2-(cyclopropanesulfonamido)thiazol-4-yl)cyclopropyl)-4-(6-ethoxypyrazin-2-yl)-2-fluorobenzamide;
N-(1-(2-(cyclopropanesulfonamido)thiazol-4-yl)cyclopropyl)-2-methyl-4-(6-(trifluoromethyl)pyrazin-2-yl)benzamide;
N-(1-(2-(cyclopropanesulfonamido)thiazol-4-yl)cyclopropyl)-4-(6-(trifluoromethyl)pyrazin-2-yl)benzamide;
N-(1-(2-(cyclopropanesulfonamido)thiazol-4-yl)-3-methoxypropyl)-4-(5-fluoropyridin-3-yl)benzamide;
N-(1-(2-(cyclopropanesulfonamido)thiazol-4-yl)-3-methoxypropyl)-4-(6-ethylpyrazin-2-yl)-2-fluorobenzamide;
N-(1-(2-(cyclopropanesulfonamido)thiazol-4-yl)-3-methoxypropyl)-2-fluoro-4-(6-(trifluoromethyl)pyrazin-2-yl)benzamide;
N-(1-(2-(cyclopropanesulfonamido)thiazol-4-yl)-3-methoxypropyl)-4-(6-ethoxypyrazin-2-yl)-2-fluorobenzamide;
N-(1-(2-(cyclopropanesulfonamido)thiazol-4-yl)-3-methoxypropyl)-2-fluoro-4-(6-isopropoxypyrazin-2-yl)benzamide;
N-(1-(2-(cyclopropanesulfonamido)thiazol-4-yl)-3-methoxypropyl)-4-(6-ethoxypyrazin-2-yl)benzamide;
N-(1-(2-(cyclopropanesulfonamido)thiazol-4-yl)ethyl)-4-(6-ethoxypyrazin-2-yl)-2-fluorobenzamide;
N-(1-(2-(cyclopropanesulfonamido)thiazol-4-yl)-3-methoxypropyl)-4-(6-ethoxypyrazin-2-yl)-2-fluorobenzamide (R enantiomer); and
N-(1-(2-(cyclopropanesulfonamido)thiazol-4-yl)-3-methoxypropyl)-4-(6-ethoxypyrazin-2-yl)-2-fluorobenzamide (S enantiomer);
   or a pharmaceutically acceptable salt and/or pharmaceutically acceptable solvate thereof.

Such CTPS1 inhibitors are disclosed in PCT publication number WO2019106146 which is incorporated by reference in its entirety for the purpose of the CTPS1 inhibitors disclosed therein. In particular a CTPS1 inhibitor may be a compound described in any one of clauses 1 to 110 of WO2019106146 or a pharmaceutically acceptable salt and/or pharmaceutically acceptable solvate thereof, in particular a compound R1 to R93 or a pharmaceutically acceptable salt and/or pharmaceutically acceptable solvate thereof.

Alternatively, the CTPS1 inhibitor is compound of formula (II):

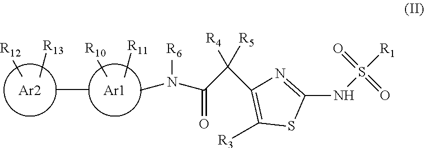

wherein
R₁ is C₁₋₅alkyl, C₀₋₂alkyleneC₃₋₅cycloalkyl which cycloalkyl is optionally substituted by
CH₃, C₁₋₃alkyleneOC₁₋₂alkyl, or CF₃;
R₃ is H, halo, CH₃, OC₁₋₂alkyl or CF₃;
   or R₃ together with R₅ forms a 5- or 6-membered cycloalkyl or 5 or 6 membered oxygen-containing heterocycloalkyl;
R₄ and R₅ are each independently H, halo, C₁₋₆alkyl, C₀₋₂alkyleneC₃₋₆cycloalkyl, C₀₋₂alkyleneC₃₋₆heterocycloalkyl, OC₁₋₆alkyl, OC₀₋₂alkyleneC₃₋₆cycloalkyl, C₁₋₃alkyleneOC₁₋₃alkyl, C₁₋₆alkylOH, C₁₋₆haloalkyl, OC₁₋₆haloalkyl or NR₂₁R₂₂,
   or R₄ is H and R₅ together with R₃ form a 5- or 6-membered cycloalkyl or 5 or 6 membered oxygen-containing heterocycloalkyl,
   or R₄ and R₅ together with the carbon atom to which they are attached form a C₃₋₆cycloalkyl or C₃₋₆heterocycloalkyl,
   or R₄ is H and R₅ and R₆ are a C₂₋₃alkylene chain forming a 5- or 6-membered ring;
   or R₄ is 0 and R₅ is absent;
R₆ is H or C₁₋₃alkyl,
   or R₆ together with R₁₁ when in the ortho-position to the amide are a C₂alkylene chain forming a 5-membered ring,
   or R₅ and R₆ are a C₂₋₃alkylene chain forming a 5- or 6-membered ring and R₄ is H;
Ar1 is 6-membered aryl or heteroaryl;
Ar2 is a 6-membered aryl or heteroaryl and is attached to Ar1 in the para position relative to the amide;
R₁₀ is H, halo, C₁₋₃alkyl, OC₁₋₂alkyl, C₁₋₂haloalkyl, OC₁₋₂haloalkyl or CN;
R₁₁ is H, F, C, CH₃, ethyl, OCH₃, CF₃, OCF₃ or CN,
   or R₁₁, when in the ortho-position to the amide, together with R₆ are a C₂alkylene chain forming a 5-membered ring;
   R₁₂ is attached to Ar2 in the ortho or meta position relative to Ar1 and R₁₂ is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR₂₃R₂₄, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2, or R₁₂ together with a nitrogen atom to which it is attached forms an N-oxide (N^{+—O—);}
   R₁₃ is H, halo, CH₃ or OCH₃;
   R₂₁ is H, C₁₋₅alkyl, C(O)C₁₋₅alkyl, C(O)OC₁₋₅alkyl;
   R₂₂ is H or CH₃;
   R₂₃ is H or C₁₋₂alkyl; and
   R₂₄ is H or C₁₋₂alkyl;
or a pharmaceutically acceptable salt and/or pharmaceutically acceptable solvate thereof.

More suitably the CTPS1 inhibitor is selected from the following (‘List B’) compounds:
N-([1,1′-biphenyl]-4-yl)-2-(2-(methylsulfonamido)thiazol-4-yl)acetamide;
N-([1,1′-biphenyl]-4-yl)-2-(2-(cyclopropanesulfonamido)thiazol-4-yl)acetamide;
2-(2-(cyclopropanesulfonamido)thiazol-4-yl)-2-ethyl-N-(5-(pyrazin-2-yl)pyridin-2-yl)butanamide;
2-(2-(cyclopropanesulfonamido)thiazol-4-yl)-2-methyl-N-(4-(pyrimidin-2-yl)phenyl)propanamide;
2-(2-(cyclopropanesulfonamido)thiazol-4-yl)-N-(4-(pyridin-3-yl)phenyl)butanamide (racemic);
(R)-2-(2-(cyclopropanesulfonamido)thiazol-4-yl)-N-(4-(pyridin-3-yl)phenyl)butanamide;
(S)-2-(2-(cyclopropanesulfonamido)thiazol-4-yl)-N-(4-(pyridin-3-yl)phenyl)butanamide;
2-(2-(cyclopropanesulfonamido)thiazol-4-yl)-N-(4-(5-fluoropyridin-3-yl)phenyl)butanamide (racemic);
(R)-2-(2-(cyclopropanesulfonamido)thiazol-4-yl)-N-(4-(5-fluoropyridin-3-yl)phenyl)butanamide;
(S)-2-(2-(cyclopropanesulfonamido)thiazol-4-yl)-N-(4-(5-fluoropyridin-3-yl)phenyl)butanamide;
2-(2-(cyclopropanesulfonamido)thiazol-4-yl)-3-methyl-N-(4-(pyrimidin-5-yl)phenyl)butanamide;
2-(2-(cyclopropanesulfonamido)thiazol-4-yl)-3-methyl-N-(4-(pyridin-3-yl)phenyl)butanamide;
2-(2-(cyclopropanesulfonamido)thiazol-4-yl)-N-(4-(5-methoxypyridin-3-yl)phenyl)-2-methylpropanamide;
N-(2-chloro-4-(pyridin-3-yl)phenyl)-2-(2-(cyclopropanesulfonamido)thiazol-4-yl)acetamide;
2-(2-(cyclopropanesulfonamido)-5-methylthiazol-4-yl)-2-methyl-N-(4-(6-(trifluoromethyl)pyrazin-2-yl)phenyl)propanamide;
2-(2-(cyclopropanesulfonamido)thiazol-4-yl)-2-methyl-N-(4-(pyrimidin-5-yl)phenyl)propanamide;
6-(4-(2-(2-(cyclopropanesulfonamido)thiazol-4-yl)-2-methylpropanamido)phenyl)-N,N-dimethylpyrazine-2-carboxamide:
N-(5-(5-cyanopyridin-3-yl)pyrimidin-2-yl)-2-(2-(cyclopropanesulfonamido)thiazol-4-yl)-2-methylpropanamide;
N-([1,1′-biphenyl]-4-yl)-2-(5-chloro-2-(cyclopropanesulfonamido)thiazol-4-yl)acetamide;
2-(2-(cyclopropanesulfonamido)thiazol-4-yl)-N-(4-(6-ethynylpyrazin-2-yl)phenyl) butanamide;
2-(2-(cyclopropanesulfonamido)thiazol-4-yl)-N-(6-(pyrimidin-5-yl)pyridin-3-yl)acetamide;
2-(2-(cyclopropanesulfonamido)thiazol-4-yl)-N-(5-phenylpyridin-2-yl)acetamide;
2-(2-(cyclopropanesulfonamido)thiazol-4-yl)-N-(4′-fluoro-[1,1′-biphenyl]-4-yl)acetamide;
2-(2-(cyclopropanesulfonamido)thiazol-4-yl)-N-methyl-N-(4-(pyridin-3-yl)phenyl)acetamide;
N-([2,3′-bipyridin]-5-yl)-2-(2-(cyclopropanesulfonamido)thiazol-4-yl)propanamide;
2-(2-(cyclopropanesulfonamido)thiazol-4-yl)-N-(3′-methoxy-[1,1′-biphenyl]-4-yl)-2-methylpropanamide;
2-(2-(cyclopropanesulfonamido)thiazol-4-yl)-2-methyl-N-(4-(pyridin-3-yl)phenyl)propanamide;
2-(2-(cyclopropanesulfonamido)thiazol-4-yl)-2-methyl-N-(4-(5-methylpyridin-3-yl)phenyl)propanamide;
2-(2-(cyclopropanesulfonamido)thiazol-4-yl)-2-methyl-N-(4-(pyridazin-4-yl)phenyl)propanamide;
2-(2-(cyclopropanesulfonamido)thiazol-4-yl)-2-methyl-N-(4-(pyrazin-2-yl)phenyl)propanamide;
2-(2-(cyclopropanesulfonamido)thiazol-4-yl)-N-(4-(6-methoxypyrazin-2-yl)phenyl)butanamide;
N-(3-cyano-4-(pyrazin-2-yl)phenyl)-2-(2-(cyclopropanesulfonamido)thiazol-4-yl)-2-methylpropanamide;
2-(2-(cyclopropanesulfonamido)thiazol-4-yl)-2-methyl-N-(5-(6-(trifluoromethyl)pyrazin-2-yl)pyridin-2-yl)propanamide;
2-(2-(cyclopropanesulfonamido)thiazol-4-yl)-N-(2,3-difluoro-4-(pyridin-3-yl)phenyl)-2-methylpropanamide;
2-(2-(cyclopropanesulfonamido)thiazol-4-yl)-2-methyl-N-(5-(pyridin-3-yl)pyrimidin-2-yl)propanamide;
2-(2-(cyclopropanesulfonamido)thiazol-4-yl)-2-methyl-N-(5-(6-propoxypyrazin-2-yl)pyridin-2-yl)propanamide;
2-(2-(cyclopropanesulfonamido)thiazol-4-yl)-N-(3-fluoro-4-(pyrazin-2-yl)phenyl)-2-methylpropanamide;
2-(2-(cyclopropanesulfonamido)thiazol-4-yl)-2-methyl-N-(4-(pyridin-3-yl)-2-(trifluoromethoxy)phenyl)propanamide;
N-(2-chloro-4-(pyridin-3-yl)phenyl)-2-(2-(cyclopropanesulfonamido)thiazol-4-yl)-2-methylpropanamide;
2-(2-(cyclopropanesulfonamido)thiazol-4-yl)-N-(2-fluoro-4-(pyridin-3-yl)phenyl)-2-methylpropanamide;
2-(2-(cyclopropanesulfonamido)thiazol-4-yl)-N-(3-methoxy-4-(pyrazin-2-yl)phenyl)-2-methylpropanamide;
2-(2-(cyclopropanesulfonamido)thiazol-4-yl)-N-(4-(2-methoxypyridin-3-yl)phenyl)-2-methylpropanamide;
2-(2-(cyclopropanesulfonamido)thiazol-4-yl)-N-(4-(5-(hydroxymethyl)pyridin-3-yl)phenyl)acetamide;
2-(2-(cyclopropanesulfonamido)thiazol-4-yl)-N-(4-(6-methoxypyridin-3-yl)phenyl)-2-methylpropanamide;
N-(4-(5-cyanopyridin-3-yl)phenyl)-2-(2-(cyclopropanesuIfonamido)thiazol-4-yl)-2-methylpropanamide;
2-(2-(cyclopropanesulfonamido)thiazol-4-yl)-2-methyl-N-(4-(5-(trifluoromethyl)pyridin-3-yl)phenyl)propanamide;
2-(2-(cyclopropanesulfonamido)thiazol-4-yl)-2-methyl-N-(4-(5-(methylsulfonyl)pyridin-3-yl)phenyl)propanamide;
2-(2-(cyclopropanesulfonamido)thiazol-4-yl)-N-(2-methoxy-4-(5-methoxypyridin-3-yl)phenyl)-2-methylpropanamide;
2-(2-(cyclopropanesulfonamido)thiazol-4-yl)-2-methyl-N-(5′-(trifluoromethyl)-[3,3′-bipyridin]-6-yl)propanamide;
2-(2-(cyclopropanesulfonamido)thiazol-4-yl)-N-(4-(6-ethoxypyrazin-2-yl)phenyl)-2-methylpropanamide;
2-(2-(cyclopropanesulfonamido)thiazol-4-yl)-2-methyl-N-(4-(6-morpholinopyrazin-2-yl)phenyl)propanamide;
N-(4-(6-cyclobutoxypyrazin-2-yl)phenyl)-2-(2-(cyclopropanesulfonamido)thiazol-4-yl)-2-methylpropanamide;
2-(2-(cyclopropanesulfonamido)thiazol-4-yl)-2-methyl-N-(4-(6-propoxypyrazin-2-yl)phenyl)propanamide;
2-(2-(cyclopropanesulfonamido)thiazol-4-yl)-N-(2-fluoro-4-(5-methoxypyridin-3-yl)phenyl)-2-methylpropanamide;
2-(2-(cyclopropanesulfonamido)thiazol-4-yl)-2-methoxy-N-(4-(6-methoxypyrazin-2-yl)phenyl)acetamide;
N-(4-(5-chloropyridin-3-yl)phenyl)-2-(2-(cyclopropanesulfonamido)thiazol-4-yl)-2-isopropoxyacetamide;
2-(2-(cyclopropanesulfonamido)thiazol-4-yl)-4-methoxy-N-(5-(6-(trifluoromethyl)pyrazin-2-yl)pyridin-2-yl)butanamide;
N-([1,1′-biphenyl]-4-yl)-2-(2-(cyclopropanesulfonamido)thiazol-4-yl)-2,2-difluoroacetamide;
2-(2-(cyclobutanesulfonamido)thiazol-4-yl)-N-(4-(6-methoxypyrazin-2-yl)phenyl)acetamide;
N-([3,3′-bipyridin]-6-yl)-2-(2-(cyclopropanesulfonamido)thiazol-4-yl)butanamide;
2-(2-(cyclopropanesulfonamido)thiazol-4-yl)-2-methyl-N-(5-phenylpyridin-2-yl)propanamide;
2-(2-(cyclopropanesulfonamido)thiazol-4-yl)-N-(5-(pyrimidin-5-yl)pyridin-2-yl)acetamide;
N-([3,3′-bipyridin]-6-yl)-2-(2-(cyclopropanesulfonamido)thiazol-4-yl)acetamide;
2-(2-(cyclopropanesulfonamido)thiazol-4-yl)-N-(6-phenylpyridin-3-yl)acetamide;
N-([2,3′-bipyridin]-5-yl)-2-(2-(cyclopropanesulfonamido)thiazol-4-yl)acetamide;
2-(2-(cyclopropanesulfonamido)thiazol-4-yl)-2-methyl-N-(4-(pyridazin-3-yl)phenyl)propanamide;
2-(2-(cyclopropanesulfonamido)thiazol-4-yl)-N-(4-(pyridazin-4-yl)phenyl)butanamide;
2-(2-(cyclopropanesulfonamido)thiazol-4-yl)-N-(4-(pyrazin-2-yl)phenyl)butanamide;
N-(4-(5-chloropyridin-3-yl)phenyl)-2-(2-(cyclopropanesulfonamido)thiazol-4-yl)-4-methoxybutanamide;
2-(2-(cyclopropanesulfonamido)thiazol-4-yl)-N-(4-(5-fluoropyridin-3-yl)phenyl)butanamide;
2-(2-(cyclopropanesulfonamido)thiazol-4-yl)-N-(4-(6-(trifluoromethyl)pyrazin-2-yl)phenyl)butanamide;
2-(2-(cyclopropanesulfonamido)thiazol-4-yl)-3-methyl-N-(4-(pyrazin-2-yl)phenyl)butanamide;
2-(2-(cyclopropanesulfonamido)thiazol-4-yl)-N-(4-(6-propoxypyrazin-2-yl)phenyl)butanamide;
2-(2-(cyclopropanesulfonamido)thiazol-4-yl)-N-(4-(6-isopropoxypyrazin-2-yl)phenyl)butanamide;
2-(2-(cyclopropanesulfonamido)thiazol-4-yl)-N-(4-(6-cyclopropoxypyrazin-2-yl)phenyl)butanamide;
N-(4-(6-chloropyrazin-2-yl)phenyl)-2-(2-(cyclopropanesulfonamido)thiazol-4-yl)butanamide;
N-(4-(6-cyanopyrazin-2-yl)phenyl)-2-(2-(cyclopropanesulfonamido)thiazol-4-yl)butanamide;
2-(2-(cyclopropanesulfonamido)thiazol-4-yl)-N-(4-(6-(trifluoromethyl)pyrazin-2-yl)phenyl)butanamide;
2-(2-(cyclopropanesulfonamido)thiazol-4-yl)-N-(4-(6-methoxypyrazin-2-yl)phenyl)acetamide;
2-(2-(cyclopropanesulfonamido)thiazol-4-yl)-N-(4-(pyrazin-2-yl)phenyl)acetamide;
N-([1,1′-biphenyl]-4-yl)-2-(cyclopropanesulfonamido)-4,5,6,7-tetrahydrobenzo[d]thiazole-4-carboxamide;
2-(cyclopropanesulfonamido)-N-(4-(pyridin-3-yl)phenyl)-4,5,6,7-tetrahydrobenzo[d]thiazole-4-carboxamide;
N-([1,1′-biphenyl]-4-yl)-2-(2-(cyclopropanesulfonamido)thiazol-4-yl)butanamide;
N-([1,1′-biphenyl]-4-yl)-2-(2-(cyclopropanesulfonamido)thiazol-4-yl)-3-methylbutanamide;
N-(3′-chloro-[1,1′-biphenyl]-4-yl)-2-(2-(cyclopropanesulfonamido)thiazol-4-yl)-2-methylpropanamide;
N-(3′-cyano-[1,1′-biphenyl]-4-yl)-2-(2-(cyclopropanesulfonamido)thiazol-4-yl)-2-methylpropanamide;
2-(2-(cyclopropanesulfonamido)thiazol-4-yl)-2,2-difluoro-N-(4-(pyridin-3-yl)phenyl)acetamide;
N-(4-(5-fluoropyridin-3-yl)phenyl)-2-(2-(cyclopropanesulfonamido)thiazol-4-yl)butanamide;
2-(2-(cyclopropanesulfonamido)thiazol-4-yl)-2-ethyl-N-(4-(pyridin-3-yl)phenyl)butanamide;
N-(4-(5-cyanopyridin-3-yl)phenyl)-2-(2-(cyclopropanesuIfonamido)thiazol-4-yl)-2-ethylbutanamide;
2-(2-(cyclopropanesulfonamido)thiazol-4-yl)-N-(4-(5-ethoxypyridin-3-yl)phenyl)propanamide;
N-(4-(5-chloropyridin-3-yl)phenyl)-2-(2-(cyclopropanesulfonamido)thiazol-4-yl)propanamide;
2-(2-(cyclopropanesulfonamido)thiazol-4-yl)-N-(4-(5-ethoxypyridin-3-yl)phenyl)butanamide;
N-([1,1′-biphenyl]-4-yl)-2-(2-(cyclopropanesulfonamido)thiazol-4-yl)-2-methylpropanamide;
2-(2-(cyclopropanesulfonamido)thiazol-4-yl)-N-(4-(4-methylpyridin-3-yl)phenyl)acetamide;
2-(2-(cyclopropanesulfonamido)thiazol-4-yl)-N-(4-(5-methylpyridin-3-yl)phenyl)acetamide;
2-(2-(cyclopropanesulfonamido)thiazol-4-yl)-N-(4-(2-methylpyridin-3-yl)phenyl)acetamide;
2-(2-(cyclopropanesulfonamido)thiazol-4-yl)-N-(4-(6-methylpyridin-3-yl)phenyl)acetamide;
2-(2-(cyclopropanesulfonamido)thiazol-4-yl)-N-(4-(pyridin-3-yl)phenyl) propanamide;
2-(2-(cyclopropanesulfonamido)thiazol-4-yl)-2-methyl-N-(4-(2-methylpyridin-3-yl)phenyl)propanamide;
2-(2-(cyclopropanesulfonamido)thiazol-4-yl)-2-oxo-N-(4-(pyridin-3-yl)phenyl)acetamide;
2-(2-(cyclopropanesulfonamido)thiazol-4-yl)-2-methyl-N-(4-(6-methylpyridin-3-yl)phenyl)propanamide;
2-(2-(cyclopropanesulfonamido)thiazol-4-yl)-N-(4-(6-(trifluoromethyl) pyrazin-2-yl)phenyl)butanamide;
(R)-2-(2-(cyclopropanesulfonamido)thiazol-4-yl)-2-methoxy-N-(4-(6-methoxypyrazin-2-yl)phenyl)acetamide;
(S)-2-(2-(cyclopropanesulfonamido)thiazol-4-yl)-2-methoxy-N-(4-(6-methoxypyrazin-2-yl)phenyl)acetamide;
2-(2-(cyclopropanesulfonamido)thiazol-4-yl)-2-methyl-N-(5-(6-(2,2,2-trifluoroethoxy)pyrazin-2-yl)pyridin-2-yl)propanamide;
2-(2-(cyclopropanesulfonamido)thiazol-4-yl)-N-(3-fluoro-5-(pyrazin-2-yl) pyridin-2-yl)-2-methylpropanamide;
2-(2-(cyclopropanesulfonamido)thiazol-4-yl)-N-(5-(6-ethoxypyrazin-2-yl)-3-fluoropyridin-2-yl)-2-methylpropanamide;
2-(2-(cyclopropanesulfonamido)thiazol-4-yl)-N-(3-fluoro-5-(6-(trifluoromethyl)pyrazin-2-yl)pyridin-2-yl)-2-methylpropanamide;
N-(5-(6-cyanopyrazin-2-yl)-3-fluoropyridin-2-yl)-2-(2-(cyclopropanesulfonamido)thiazol-4-yl)-2-methylpropanamide;
2-(2-(cyclopropanesulfonamido)thiazol-4-yl)-2-methyl-N-(5′-(2,2,2-trifluoroethoxy)-[3,3′-bipyridin]-6-yl)propanamide;
2-(2-(cyclopropanesulfonamido)thiazol-4-yl)-N-(5′-(difluoromethoxy)-[3,3′-bipyridin]-6-yl)-2-methylpropanamide;
N-([2,3′-bipyridin]-5-yl)-2-(2-(cyclopropanesulfonamido)thiazol-4-yl)-2-methylpropanamide;
2-(2-(cyclopropanesulfonamido)thiazol-4-yl)-2-methyl-N-(6-(pyrimidin-5-yl)pyridin-3-yl)propanamide;
2-(2-(cyclopropanesulfonamido)thiazol-4-yl)-N-(4-(5-(difluoromethoxy)pyridin-3-yl)phenyl)-2-methylpropanamide;
2-(2-(cyclopropanesulfonamido)thiazol-4-yl)-2-ethyl-N-(4-(6-methoxypyrazin-2-yl)phenyl)butanamide;
N-(4-(5-chloropyridin-3-yl)phenyl)-2-(2-(cyclopropanesulfonamido)thiazol-4-yl)-2-ethylbutanamide;
2-(2-(cyclopropanesulfonamido)thiazol-4-yl)-2-ethyl-N-(2-fluoro-4-(pyridin-3-yl)phenyl)butanamide;
2-(2-(cyclopropanesulfonamido)thiazol-4-yl)-2-ethyl-N-(4-(pyrazin-2-yl)phenyl)butanamide;
2-(2-(cyclopropanesulfonamido)thiazol-4-yl)-2-ethyl-N-(4-(6-propoxypyrazin-2-yl)phenyl)butanamide;
2-(2-(cyclopropanesulfonamido)thiazol-4-yl)-N-(2-fluoro-4-(6-(trifluoromethyl)pyrazin-2-yl)phenyl)-2-methylpropanamide;
2-(2-(cyclopropanesulfonamido)thiazol-4-yl)-N-(4-(5-ethoxypyridin-3-yl)-2-fluorophenyl)-2-methylpropanamide;
2-(2-(cyclopropanesulfonamido)thiazol-4-yl)-N-(2-fluoro-4-(5-fluoropyridin-3-yl)phenyl)-2-methylpropanamide;
2-(2-(cyclopropanesulfonamido)thiazol-4-yl)-2-methyl-N-(4-(6-(2,2,2-trifluoroethoxy)pyrazin-2-yl)phenyl)propanamide;
N-(4-(5-chloropyridin-3-yl)-2-fluorophenyl)-2-(2-(cyclopropanesulfonamido)thiazol-4-yl)-2-methylpropanamide;
N-(4-(5-cyanopyridin-3-yl)-2-fluorophenyl)-2-(2-(cyclopropanesulfonamido)thiazol-4-yl)-2-methylpropanamide;
1-(2-(cyclopropanesulfonamido)thiazol-4-yl)-N-(4-(6-methoxypyrazin-2-yl)phenyl)cyclopentane-1-carboxamide;
2-(2-(cyclopropanesulfonamido)thiazol-4-yl)-N-(2-fluoro-4-(6-(2,2,2-trifluoroethoxy)pyrazin-2-yl)phenyl)-2-methylpropanamide;
2-(2-(cyclopropanesulfonamido)thiazol-4-yl)-N-(2-fluoro-4-(5-(2,2,2-trifluoroethoxy)pyridin-3-yl)phenyl)-2-methylpropanamide;
2-(2-(cyclopropanesulfonamido)thiazol-4-yl)-N-(2-fluoro-4-(5-(trifluoromethyl)pyridin-3-yl)phenyl)-2-methylpropanamide;
2-(2-(cyclopropanesulfonamido)thiazol-4-yl)-N-(4-(6-ethoxypyrazin-2-yl)-2-fluorophenyl)-2-methylpropanamide;
2-(2-(cyclopropanesulfonamido)thiazol-4-yl)-2-methyl-N-(4-(5-(2,2,2-trifluoroethoxy)pyridin-3-yl)phenyl)propanamide;
2-(2-(cyclopropanesulfonamido)thiazol-4-yl)-N-(4-(6-ethynylpyrazin-2-yl)phenyl)-2-methylpropanamide;
2-(2-(cyclopropanesulfonamido)thiazol-4-yl)-N-(4-(6-ethoxypyrazin-2-yl)-2-methylphenyl)-2-methylpropanamide;
N-(4-(6-chloropyrazin-2-yl)-2-methylphenyl)-2-(2-(cyclopropanesulfonamido)thiazol-4-yl)-2-methylpropanamide;
2-(2-(cyclopropanesulfonamido)thiazol-4-yl)-N-(4-(5-(difluoromethoxy)pyridin-3-yl)-2-fluorophenyl)-2-methylpropanamide;
2-(2-(cyclopropanesulfonamido)thiazol-4-yl)-2-methyl-N-(5-(pyrazin-2-yl)pyridin-2-yl)propanamide;
N-(5-(6-cyclobutoxypyrazin-2-yl)pyridin-2-yl)-2-(2-(cyclopropanesulfonamido)thiazol-4-yl)-2-methylpropanamide;
2-(2-(cyclopropanesulfonamido)thiazol-4-yl)-N-(5-(6-cyclopropoxypyrazin-2-yl)pyridin-2-yl)-2-methylpropanamide;
2-(2-(cyclopropanesulfonamido)thiazol-4-yl)-N-(5-(6-isopropoxypyrazin-2-yl)pyridin-2-yl)-2-methylpropanamide;
N-([3,3′-bipyridin]-6-yl)-2-(2-(cyclopropanesulfonamido)thiazol-4-yl)-2-ethylbutanamide;
2-(2-(cyclopropanesulfonamido)thiazol-4-yl)-N-(5′-ethoxy-[3,3′-bipyridin]-6-yl)-2-ethylbutanamide;
2-(2-(cyclopropanesulfonamido)thiazol-4-yl)-2-methyl-N-(5′-propoxy-[3,3′-bipyridin]-6-yl)propanamide;
2-(2-(cyclopropanesulfonamido)thiazol-4-yl)-2-ethyl-N-(5-(6-(trifluoromethyl)pyrazin-2-yl)pyridin-2-yl)butanamide;
N-([3,3′-bipyridin]-6-yl)-2-(2-(cyclopropanesulfonamido)thiazol-4-yl)-2-methylpropanamide;
2-(2-(cyclopropanesulfonamido)thiazol-4-yl)-N-(2-methoxy-4-(pyridin-3-yl)phenyl)-2-methylpropanamide;
2-(2-(cyclopropanesulfonamido)thiazol-4-yl)-N-(3-methoxy-4-(pyridin-3-yl)phenyl)-2-methylpropanamide;
2-(2-(cyclopropanesulfonamido)thiazol-4-yl)-N-(3-fluoro-4-(pyridin-3-yl)phenyl)-2-methylpropanamide;
N-(3-cyano-4-(pyridin-3-yl)phenyl)-2-(2-(cyclopropanesulfonamido)thiazol-4-yl)-2-methylpropanamide;
N-(3-chloro-4-(pyridin-3-yl)phenyl)-2-(2-(cyclopropanesulfonamido)thiazol-4-yl)-2-methylpropanamide;
N-(4-(6-cyanopyrazin-2-yl)phenyl)-2-(2-(cyclopropanesulfonamido)thiazol-4-yl)-2-methylpropanamide;
N-(4-(6-chloropyrazin-2-yl)phenyl)-2-(2-(cyclopropanesulfonamido)thiazol-4-yl)-2-methylpropanamide;
2-(2-(cyclopropanesulfonamido)thiazol-4-yl)-2-ethyl-N-(4-(5-fluoropyridin-3-yl)phenyl)butanamide;
2-(2-(cyclopropanesulfonamido)thiazol-4-yl)-2-methyl-N-(4-(5-propoxypyridin-3-yl)phenyl)propanamide;
2-(2-(cyclopropanesulfonamido)thiazol-4-yl)-N-(4-(5-isopropoxypyridin-3-yl)phenyl)-2-methylpropanamide;
2-(2-(cyclopropanesulfonamido)thiazol-4-yl)-N-(2-fluoro-4-(5-isopropoxypyridin-3-yl)phenyl)-2-methylpropanamide;
N-(4-(6-chloropyrazin-2-yl)phenyl)-2-(2-(cyclopropanesulfonamido)thiazol-4-yl)-2-ethylbutanamide;
N-(4-(6-cyanopyrazin-2-yl)phenyl)-2-(2-(cyclopropanesulfonamido)thiazol-4-yl)-2-ethylbutanamide;
2-methyl-2-(2-(methylsulfonamido)thiazol-4-yl)-N-(4-(pyridin-3-yl)phenyl)propanamide;
2-(2-(cyclopropanesulfonamido)thiazol-4-yl)-N,2-dimethyl-N-(4-(pyridin-3-yl)phenyl)propanamide;
2-(cyclopropanesulfonamido)-N-(5-(6-(trifluoromethyl)pyrazin-2-yl)pyridin-2-yl)-5,6-dihydro-4H-cyclopenta[d]thiazole-4-carboxamide;
2-(2-(cyclopropanesulfonamido)thiazol-4-yl)-4-methoxy-N-(5-(pyrazin-2-yl)pyridin-2-yl)butanamide;
2-(2-(cyclopropanesulfonamido)thiazol-4-yl)-4-methoxy-N-(5′-methoxy-[3,3′-bipyridin]-6-yl)butanamide;
2-(2-(cyclopropanesulfonamido)thiazol-4-yl)-2-isopropoxy-N-(5-(6-(trifluoromethyl)pyrazin-2-yl)pyridin-2-yl)acetamide;
2-(2-(cyclopropanesulfonamido)thiazol-4-yl)-N-(5-(6-propoxypyrazin-2-yl)pyridin-2-yl)butanamide;
2-(2-(cyclopropanesulfonamido)thiazol-4-yl)-N-(5-(6-isopropoxypyrazin-2-yl)pyridin-2-yl)butanamide;
2-(2-(cyclopropanesulfonamido)thiazol-4-yl)-N-(5-(6-(trifluoromethyl)pyrazin-2-yl)pyridin-2-yl)butanamide;
2-(2-(cyclopropanesulfonamido)thiazol-4-yl)-N-(5-(6-methoxypyrazin-2-yl)pyridin-2-yl)butanamide;
2-(2-(cyclopropanesulfonamido)thiazol-4-yl)-N-(5-(6-ethoxypyrazin-2-yl)pyridin-2-yl)butanamide;
N-(5-(6-cyanopyrazin-2-yl)pyridin-2-yl)-2-(2-(cyclopropanesulfonamido)thiazol-4-yl)butanamide;
2-(2-(cyclopropanesulfonamido)thiazol-4-yl)-N-(5′-fluoro-[3,3′-bipyridin]-6-yl)butanamide;
N-(5′-cyano-[3,3′-bipyridin]-6-yl)-2-(2-(cyclopropanesulfonamido)thiazol-4-yl)butanamide;
2-(2-(cyclopropanesulfonamido)thiazol-4-yl)-N-(5-phenylpyridin-2-yl)butanamide;
2-(2-(cyclopropanesulfonamido)thiazol-4-yl)-N-(5-(6-(2,2,2-trifluoroethoxy)pyrazin-2-yl)pyridin-2-yl)butanamide;
2-(2-(cyclopropanesulfonamido)thiazol-4-yl)-N-(5-(6-ethoxypyrazin-2-yl)-3-fluoropyridin-2-yl)butanamide;
N-(5-(6-cyanopyrazin-2-yl)-3-fluoropyridin-2-yl)-2-(2-(cyclopropanesulfonamido)thiazol-4-yl)butanamide;
2-(2-(cyclopropanesulfonamido)thiazol-4-yl)-N-(5′-(2,2,2-trifluoroethoxy)-[3,3′-bipyridin]-6-yl)butanamide;
2-(2-(cyclopropanesulfonamido)thiazol-4-yl)-N-(5′-(difluoromethoxy)-[3,3′-bipyridin]-6-yl)butanamide;
N-(5-(6-chloropyrazin-2-yl)pyridin-2-yl)-2-(2-(cyclopropanesulfonamido)thiazol-4-yl)butanamide;
2-(2-(cyclopropanesulfonamido)thiazol-4-yl)-N-(2,3-difluoro-4-(pyridin-3-yl)phenyl)acetamide;
2-(2-(cyclopropanesulfonamido)thiazol-4-yl)-N-(4-(6-ethoxypyrazin-2-yl)phenyl)butanamide (racemic);
2-(2-(cyclopropanesulfonamido)thiazol-4-yl)-4-methoxy-N-(4-(6-methoxypyrazin-2-yl)phenyl)butanamide;
2-(2-(cyclopropanesulfonamido)thiazol-4-yl)-N-(4-(5-fluoropyridin-3-yl)phenyl)-4-methoxybutanamide;
2-(2-(cyclopropanesulfonamido)thiazol-4-yl)-N-(4-(6-ethoxypyrazin-2-yl)phenyl)propanamide;
2-(2-(cyclopropanesulfonamido)thiazol-4-yl)-N-(4-(5-(trifluoromethyl)pyridin-3-yl)phenyl)butanamide;
N-(4-(5-cyanopyridin-3-yl)phenyl)-2-(2-(cyclopropanesulfonamido)thiazol-4-yl)butanamide;
2-(2-(cyclopropanesulfonamido)thiazol-4-yl)-N-(4-(6-(2,2,2-trifluoroethoxy)pyrazin-2-yl)phenyl)butanamide;
2-(2-(cyclopropanesulfonamido)thiazol-4-yl)-N-(2-fluoro-4-(pyrazin-2-yl)phenyl)butanamide;
2-(2-(cyclopropanesulfonamido)thiazol-4-yl)-N-(2-fluoro-4-(6-(trifluoromethyl)pyrazin-2-yl)phenyl)butanamide;
2-(2-(cyclopropanesulfonamido)thiazol-4-yl)-N-(4-(6-ethoxypyrazin-2-yl)-2-fluorophenyl)butanamide;
2-(2-(cyclopropanesulfonamido)thiazol-4-yl)-N-(4-(5-ethoxypyridin-3-yl)-2-fluorophenyl)butanamide;
2-(2-(cyclopropanesulfonamido)thiazol-4-yl)-N-(2-fluoro-4-(5-fluoropyridin-3-yl)phenyl)butanamide;
2-(2-(cyclopropanesulfonamido)thiazol-4-yl)-N-(2-fluoro-4-(pyridin-3-yl)phenyl)butanamide;
N-(4-(5-cyanopyridin-3-yl)-2-fluorophenyl)-2-(2-(cyclopropanesulfonamido)thiazol-4-yl)butanamide;
N-(4-(5-chloropyridin-3-yl)-2-fluorophenyl)-2-(2-(cyclopropanesulfonamido)thiazol-4-yl)butanamide;
(R)-2-(2-(cyclopropanesulfonamido)thiazol-4-yl)-N-(4-(6-ethoxypyrazin-2-yl)phenyl)butanamide;
(S)-2-(2-(cyclopropanesulfonamido)thiazol-4-yl)-N-(4-(6-ethoxypyrazin-2-yl)phenyl)butanamide;
N-(4-(1-(5-(6-ethoxypyrazin-2-yl)indolin-1-yl)-1-oxobutan-2-yl)thiazol-2-yl)cyclopropanesulfonamide;
2-(2-(cyclopropanesulfonamido)thiazol-4-yl)-N-(2-fluoro-4-(6-(2,2,2-trifluoroethoxy)pyrazin-2-yl)phenyl)butanamide;
2-(2-(cyclopropanesulfonamido)thiazol-4-yl)-N-(2-fluoro-4-(6-methoxypyrazin-2-yl)phenyl)butanamide;
2-(2-(cyclopropanesulfonamido)thiazol-4-yl)-N-(4-(5-(difluoromethoxy)pyridin-3-yl)-2-fluorophenyl)butanamide;
2-(2-(cyclopropanesulfonamido)thiazol-4-yl)-N-(4-(5-(difluoromethoxy)pyridin-3-yl)phenyl)butanamide;
2-(2-(cyclopropanesulfonamido)thiazol-4-yl)-N-(4-(5-(2,2,2-trifluoroethoxy)pyridin-3-yl)phenyl)butanamide;
2-(2-(cyclopropanesulfonamido)thiazol-4-yl)-N-(2-fluoro-4-(5-(2,2,2-trifluoroethoxy)pyridin-3-yl)phenyl)butanamide;
2-(cyclopropanesulfonamido)-N-(4-(pyridin-3-yl)phenyl)-5,6-dihydro-4H-cyclopenta[d]thiazole-4-carboxamide;
2-(2-(cyclopropanesulfonamido)thiazol-4-yl)-2-methoxy-N-(5-(6-(trifluoromethyl)pyrazin-2-yl) pyridin-2-yl)acetamide;
2-(2-(cyclopropanesulfonamido)thiazol-4-yl)-N-(2-methoxy-4-(pyridin-3-yl)phenyl)acetamide;
2-(2-(cyclopropanesulfonamido)thiazol-4-yl)-N-(2-fluoro-4-(pyridin-3-yl)phenyl)acetamide;
N-(4-(5-cyanopyridin-3-yl)phenyl)-2-(cyclopropanesulfonamido)-5,6-dihydro-4H-cyclopenta[d]thiazole-4-carboxamide;
2-(cyclopropanesulfonamido)-N-(4-(5-fluoropyridin-3-yl)phenyl)-5,6-dihydro-4H-cyclopenta[d]thiazole-4-carboxamide;
2-(2-(cyclopropanesulfonamido)thiazol-4-yl)-2-methoxy-N-(4-(pyridin-3-yl)phenyl)acetamide;
2-(2-(cyclopropanesulfonamido)thiazol-4-yl)-N-(2-fluoro-4-(pyridin-3-yl)phenyl)-2-methoxyacetamide;
N-(2-chloro-4-(pyridin-3-yl)phenyl)-2-(2-(cyclopropanesulfonamido)thiazol-4-yl)butanamide;
2-(2-(cyclopropanesulfonamido)thiazol-4-yl)-N-(4-(6-(2,2,2-trifluoroethoxy)pyrazin-2-yl)phenyl)butanamide;
2-(2-(cyclopropanesulfonamido)thiazol-4-yl)-N-(4-(6-(2,2,2-trifluoroethoxy)pyrazin-2-yl)phenyl)butanamide;
2-(2-(cyclopropanesulfonamido)thiazol-4-yl)-N-(5′-methoxy-[3,3′-bipyridin]-6-yl)-2-methylpropanamide;
N-(5′-chloro-[3,3′-bipyridin]-6-yl)-2-(2-(cyclopropanesulfonamido)thiazol-4-yl)-2-methylpropanamide;
N-(5′-cyano-[3,3′-bipyridin]-6-yl)-2-(2-(cyclopropanesulfonamido)thiazol-4-yl)-2-methylpropanamide;
2-(2-(cyclopropanesulfonamido)thiazol-4-yl)-N-(5-fluoro-[3,3′-bipyridin]-6-yl)-2-methylpropanamide;
N-(5′-cyano-5-fluoro-[3,3′-bipyridin]-6-yl)-2-(2-(cyclopropanesulfonamido)thiazol-4-yl)-2-methylpropanamide;
N-(5′-chloro-5-fluoro-[3,3′-bipyridin]-6-yl)-2-(2-(cyclopropanesulfonamido)thiazol-4-yl)-2-methylpropanamide;
2-(2-(cyclopropanesulfonamido)thiazol-4-yl)-N-(5,5′-difluoro-[3,3′-bipyridin]-6-yl)-2-methylpropanamide;
N-(5-(3-chloro-5-methylphenyl)pyridin-2-yl)-2-(2-(cyclopropanesulfonamido)thiazol-4-yl)-2-methylpropanamide;
2-(2-(cyclopropanesulfonamido)thiazol-4-yl)-N-(5-(3-methoxyphenyl)pyridin-2-yl)-2-methylpropanamide;
2-(2-(cyclopropanesulfonamido)thiazol-4-yl)-N-(5-(3-fluoro-5-methoxyphenyl)pyridin-2-yl)-2-methylpropanamide;
2-(2-(cyclopropanesulfonamido)thiazol-4-yl)-N-(5-(3,5-dimethoxyphenyl)pyridin-2-yl)-2-methylpropanamide;
2-(2-(cyclopropanesulfonamido)thiazol-4-yl)-2-methyl-N-(5-(3-(trifluoromethyl)phenyl)pyridin-2-yl)propanamide;
2-(2-(cyclopropanesulfonamido)thiazol-4-yl)-2-methyl-N-(5-(3-(trifluoromethoxy)phenyl)pyridin-2-yl)propanamide;
2-(2-(cyclopropanesulfonamido)thiazol-4-yl)-N-(5-(3-(2-hydroxypropan-2-yl)phenyl)pyridin-2-yl)-2-methylpropanamide;
2-(2-(cyclopropanesulfonamido)thiazol-4-yl)-2-methyl-N-(5-(3-morpholinophenyl)pyridin-2-yl)propanamide;
2-(2-(cyclopropanesulfonamido)thiazol-4-yl)-2-methyl-N-(6-phenylpyridin-3-yl)propanamide;
2-(2-(cyclopropanesulfonamido)thiazol-4-yl)-N-(4-(2-fluoropyridin-3-yl)phenyl)-2-methylpropanamide;
2-(2-(cyclopropanesulfonamido)thiazol-4-yl)-N-(4-(5-(hydroxymethyl)pyridin-3-yl)phenyl)-2-methylpropanamide;
2-(2-(cyclopropanesulfonamido)thiazol-4-yl)-N-(4-(2-methoxypyrimidin-5-yl)phenyl)acetamide;
N-(4′-(tert-butyl)-[1,1′-biphenyl]-4-yl)-2-(2-(cyclopropanesulfonamido)thiazol-4-yl)acetamide;
2-(2-(cyclopropanesulfonamido)thiazol-4-yl)-N-(4-(pyridin-3-yl)phenyl)acetamide;
2-(2-(cyclopropanesulfonamido)thiazol-4-yl)-N-(4-(pyridin-4-yl)phenyl)acetamide;
2-(2-(cyclopropanesulfonamido)thiazol-4-yl)-N-(2′-methoxy-[1,1′-biphenyl]-4-yl)acetamide;
2-(2-(cyclopropanesulfonamido)thiazol-4-yl)-N-(4-(pyrimidin-5-yl)phenyl)acetamide;
2-(2-(cyclopropanesulfonamido)thiazol-4-yl)-2-methyl-N-(4-(2-(trifluoromethyl)pyridin-3-yl)phenyl)propanamide;
2-(2-(cyclopropanesulfonamido)thiazol-4-yl)-2-methyl-N-(5′-methyl-[3,3′-bipyridin]-6-yl)propanamide;
2-(2-(cyclopropanesulfonamido)thiazol-4-yl)-N-(4-(2-methoxy-4-methylpyridin-3-yl)phenyl)-2-methylpropanamide;
2-(2-(cyclopropanesulfonamido)thiazol-4-yl)-N-(4-(6-methoxy-5-methylpyridin-3-yl)phenyl)-2-methylpropanamide;
N-(4-(5-chloropyridin-3-yl)phenyl)-2-(2-(cyclopropanesulfonamido)thiazol-4-yl)-2-methylpropanamide;
2-(2-(cyclopropanesulfonamido)thiazol-4-yl)-N-(4-(5-fluoropyridin-3-yl)phenyl)-2-methylpropanamide;
2-(2-(cyclopropanesulfonamido)thiazol-4-yl)-2-methyl-N-(4-(4-methylpyridin-3-yl)phenyl)propanamide;
2-(2-(cyclopropanesulfonamido)thiazol-4-yl)-2-methyl-N-(4-(4-(trifluoromethyl)pyridin-3-yl)phenyl)propanamide;
N-(4-(5-chloropyridin-3-yl)-2-methoxyphenyl)-2-(2-(cyclopropanesulfonamido)thiazol-4-yl)-2-methylpropanamide;
2-(2-(cyclopropanesulfonamido)thiazol-4-yl)-N-(4-(5-(dimethylamino)pyridin-3-yl)phenyl)-2-methylpropanamide;
2-(2-(cyclopropanesulfonamido)thiazol-4-yl)-N-(2-methoxy-4-(5-methylpyridin-3-yl)phenyl)-2-methylpropanamide;
2-(2-(cyclopropanesulfonamido)thiazol-4-yl)-N-(2-methoxy-4-(5-(trifluoromethyl)pyridin-3-yl)phenyl)-2-methylpropanamide;
2-(2-(cyclopropanesulfonamido)thiazol-4-yl)-N-(4-(6-methoxypyridin-3-yl)phenyl)acetamide;
2-(2-(cyclopropanesulfonamido)thiazol-4-yl)-N-(5′-fluoro-[3,3′-bipyridin]-6-yl)-2-methylpropanamide;
N-(5-(6-chloropyrazin-2-yl)pyridin-2-yl)-2-(2-(cyclopropanesulfonamido)thiazol-4-yl)-2-methylpropanamide;
N-(5-(6-cyanopyrazin-2-yl)pyridin-2-yl)-2-(2-(cyclopropanesulfonamido)thiazol-4-yl)-2-methylpropanamide;
2-(2-(cyclopropanesulfonamido)thiazol-4-yl)-2-methyl-N-(5-(pyrimidin-5-yl)pyridin-2-yl)propanamide;
2-(2-(cyclopropanesulfonamido)thiazol-4-yl)-N-(4-(6-methoxypyrazin-2-yl)phenyl)-2-methylpropanamide;
2-(2-(cyclopropanesulfonamido)thiazol-4-yl)-2-methyl-N-(4-(6-methylpyrazin-2-yl)phenyl)propanamide;
2-(2-(cyclopropanesulfonamido)thiazol-4-yl)-2-methyl-N-(4-(6-(trifluoromethyl)pyrazin-2-yl)phenyl)propanamide;
N-(4-(6-chloropyridin-2-yl)phenyl)-2-(2-(cyclopropanesulfonamido)thiazol-4-yl)-2-methylpropanamide;
2-(2-(cyclopropanesulfonamido)thiazol-4-yl)-N-(4-(6-methoxypyridin-2-yl)phenyl)-2-methylpropanamide;
2-(2-(cyclopropanesulfonamido)thiazol-4-yl)-2-methyl-N-(4-(6-(trifluoromethyl)pyridin-2-yl)phenyl)propanamide;
2-(2-(cyclopropanesulfonamido)thiazol-4-yl)-N-(4-(4-methoxypyridin-2-yl)phenyl)-2-methylpropanamide;
2-(2-(cyclopropanesulfonamido)thiazol-4-yl)-N-(4-(6-isopropoxypyrazin-2-yl)phenyl)-2-methylpropanamide;
2-(2-(cyclopropanesulfonamido)thiazol-4-yl)-N-(4-(6-cyclopropoxypyrazin-2-yl)phenyl)-2-methylpropanamide;
2-(2-(cyclopropanesulfonamido)thiazol-4-yl)-N-(2-fluoro-4-(pyrazin-2-yl)phenyl)-2-methylpropanamide;
2-(2-(cyclopropanesulfonamido)thiazol-4-yl)-N-(2-fluoro-4-(6-methoxypyrazin-2-yl)phenyl)-2-methylpropanamide;
N-(4-(6-chloro-3-methylpyrazin-2-yl)phenyl)-2-(2-(cyclopropanesulfonamido)thiazol-4-yl)-2-methylpropanamide;
N-(4-(6-chloro-5-methylpyrazin-2-yl)phenyl)-2-(2-(cyclopropanesulfonamido)thiazol-4-yl)-2-methylpropanamide;
2-(2-(cyclopropanesulfonamido)thiazol-4-yl)-2-methyl-N-(4-(6-(pyrrolidin-1-yl)pyrazin-2-yl)phenyl)propanamide;
2-(2-(cyclopropanesulfonamido)thiazol-4-yl)-N-(4-(6-(2-(dimethylamino)ethoxy)pyrazin-2-yl)phenyl)-2-methylpropanamide;
2-(2-(cyclopropanesulfonamido)thiazol-4-yl)-2-methyl-N-(4-(3-methylpyrazin-2-yl)phenyl)propanamide;
N-(4-(6-acetamidopyrazin-2-yl)phenyl)-2-(2-(cyclopropanesulfonamido)thiazol-4-yl)-2-methylpropanamide;
2-(2-(cyclopropanesulfonamido)thiazol-4-yl)-N-(4-(5,6-dimethylpyrazin-2-yl)phenyl)-2-methylpropanamide;
2-(2-(cyclopropanesulfonamido)thiazol-4-yl)-N-(4-(6-(hydroxymethyl)pyrazin-2-yl)phenyl)-2-methylpropanamide;
2-(2-(cyclopropanesulfonamido)thiazol-4-yl)-N-(4-(3,6-dimethylpyrazin-2-yl)phenyl)-2-methylpropanamide;
2-(2-(cyclopropanesulfonamido)thiazol-4-yl)-N-(4-(5-methoxypyridin-3-yl)-2-methylphenyl)-2-methylpropanamide;
N-(4-(5-cyanopyridin-3-yl)-2-methylphenyl)-2-(2-(cyclopropanesulfonamido)thiazol-4-yl)-2-methylpropanamide;
2-(2-(cyclopropanesulfonamido)thiazol-4-yl)-N-(4-(5-fluoropyridin-3-yl)-2-methylphenyl)-2-methylpropanamide;
N-(4-(5-chloropyridin-3-yl)-3-methylphenyl)-2-(2-(cyclopropanesuIfonamido)thiazol-4-yl)-2-methylpropanamide;
N-(4-(5-cyanopyridin-3-yl)-3-ethoxyphenyl)-2-(2-(cyclopropanesulfonamido)thiazol-4-yl)-2-methylpropanamide;
2-(2-(cyclopropanesulfonamido)thiazol-4-yl)-N-(4-(5-ethoxypyridin-3-yl)phenyl)-2-methylpropanamide;
2-(2-(cyclopropanesulfonamido)thiazol-4-yl)-N-(4-(6-cyclopropylpyrazin-2-yl)phenyl)butanamide;
2-(2-(cyclopropanesulfonamido)thiazol-4-yl)-N-(5-(5-methoxypyridin-3-yl)pyrimidin-2-yl)-2-methylpropanamide;
2-(2-(cyclopropanesulfonamido)thiazol-4-yl)-N-(5-(5-fluoropyridin-3-yl)pyrimidin-2-yl)-2-methylpropanamide;
2-(2-(cyclopropanesulfonamido)thiazol-4-yl)-2-methyl-N-(5-(5-(trifluoromethyl)pyridin-3-yl)pyrimidin-2-yl)propanamide;
N-(5-(6-ethoxypyrazin-2-yl) pyridin-2-yl)-2-methyl-2-(2-((2-methylpropyl)sulfonamido)thiazol-4-yl)propanamide;
N-(4-(6-ethoxypyrazin-2-yl)phenyl)-2-methyl-2-(2-((trifluoromethyl)sulfonamido)thiazol-4-yl)propanamide;
2-methyl-2-(2-((1-methylethyl)sulfonamido)thiazol-4-yl)-N-(4-(pyridin-3-yl)phenyl)propanamide;
N-(4-(6-ethoxypyrazin-2-yl)phenyl)-2-methyl-2-(2-((1-methylethyl)sulfonamido)thiazol-4-yl)propanamide;
2-methyl-2-(2-((1-methylcyclopropane)-1-sulfonamido)thiazol-4-yl)-N-(4-(pyridin-3-yl)phenyl)propanamide;
N-(4-(5-chloropyridin-3-yl)phenyl)-2-methyl-2-(2-((1-methylcyclopropane)-1-sulfonamido)thiazol-4-yl)propanamide;
N-(4-(6-ethoxypyrazin-2-yl)phenyl)-2-methyl-2-(2-((1-methylcyclopropane)-1-sulfonamido)thiazol-4-yl)propanamide;
2-methyl-2-(2-((1-methylcyclopropane)-1-sulfonamido)thiazol-4-yl)-N-(4-(6-(trifluoromethyl)pyrazin-2-yl)phenyl)propanamide;
2-(2-((1,1-dimethylethyl)sulfonamido)thiazol-4-yl)-2-methyl-N-(4-(pyridin-3-yl)phenyl)propanamide;
2-(2-((1,1-dimethylethyl)sulfonamido)thiazol-4-yl)-N-(4-(6-ethoxypyrazin-2-yl)phenyl)-2-methylpropanamide;
2-(2-((1,1-dimethylethyl)sulfonamido)thiazol-4-yl)-2-methyl-N-(4-(6-(trifluoromethyl)pyrazin-2-yl)phenyl)propanamide;
2-(2-(cyclobutanesulfonamido)thiazol-4-yl)-2-methyl-N-(4-(pyridin-3-yl)phenyl)propanamide;
2-(2-(cyclobutanesulfonamido)thiazol-4-yl)-N-(4-(6-ethoxypyrazin-2-yl)phenyl)-2-methylpropanamide;
2-(2-(cyclobutanesulfonamido)thiazol-4-yl)-2-methyl-N-(4-(6-(trifluoromethyl)pyrazin-2-yl)phenyl)propanamide;
N-(4-(5-cyanopyridin-3-yl)phenyl)-2-(2-(cyclopropanesuIfonamido)thiazol-4-yl)-N,2-dimethylpropanamide;
2-(2-(cyclopropanesulfonamido)thiazol-4-yl)-N,2-dimethyl-N-(4-(6-(trifluoromethyl)pyrazin-2-yl)phenyl)propanamide;
2-methyl-2-(2-((2-methylpropyl)sulfonamido)thiazol-4-yl)-N-(4-(pyridin-3-yl)phenyl)propanamide;
N-(4-(6-ethoxypyrazin-2-yl)phenyl)-2-methyl-2-(2-((2-methylpropyl)sulfonamido)thiazol-4-yl)propanamide;
2-methyl-2-(2-((2-methylpropyl)sulfonamido)thiazol-4-yl)-N-(4-(6-(trifluoromethyl)pyrazin-2-yl)phenyl)propanamide;
N-(4-(5-chloropyridin-3-yl)phenyl)-2-(2-(cyclopropanesulfonamido)-5-methylthiazol-4-yl)-2-methylpropanamide;
2-(2-(cyclopropanesulfonamido)thiazol-4-yl)-N-methyl-N-(4-(pyridin-3-yl)phenyl) butanamide;
N-(4-(5-cyanopyridin-3-yl)phenyl)-2-(2-(cyclopropanesulfonamido)thiazol-4-yl)-N-methylbutanamide;
2-(2-(cyclopropanesulfonamido)-5-methylthiazol-4-yl)-N-(4-(6-ethoxypyrazin-2-yl)phenyl)-2-methylpropanamide;
N-(4-(5-chloropyridin-3-yl)phenyl)-2-(2-(cyclopropanesulfonamido)thiazol-4-yl)-N,2-dimethylpropanamide;
2-(2-(cyclopropanesulfonamido)-5-methylthiazol-4-yl)-2-methyl-N-(4-(pyridin-3-yl)phenyl)propanamide;
N-(4-(5-cyanopyridin-3-yl)-2,6-dimethylphenyl)-2-(2-(cyclopropanesulfonamido)thiazol-4-yl)-2-methylpropanamide;
N-(4-(5-chloropyridin-3-yl)-2,6-dimethylphenyl)-2-(2-(cyclopropanesulfonamido)thiazol-4-yl)-2-methylpropanamide;
N-(4-(5-cyanopyridin-3-yl)-3-methylphenyl)-2-(2-(cyclopropanesulfonamido)thiazol-4-yl)-2-methylpropanamide;
2-(2-(cyclopropanesulfonamido)thiazol-4-yl)-N-(4-(pyridin-3-yl)phenyl) butanamide;
2-(2-(cyclopropanesulfonamido)thiazol-4-yl)-N-(4-(5-fluoropyridin-3-yl)phenyl) butanamide;
2-(2-(cyclopropanesulfonamido)thiazol-4-yl)-2-methoxy-N-(4-(6-methoxypyrazin-2-yl)phenyl)acetamide;
2-(2-(cyclopropanesulfonamido)thiazol-4-yl)-N-(4-(6-ethoxypyrazin-2-yl)phenyl) butanamide;
2-amino-2-(2-(cyclopropanesulfonamido)thiazol-4-yl)-N-(4-(6-ethoxypyrazin-2-yl)phenyl)acetamide;
2-acetamido-2-(2-(cyclopropanesulfonamido)thiazol-4-yl)-N-(4-(6-ethoxypyrazin-2-yl)phenyl)acetamide;
methyl(1-(2-(cyclopropanesulfonamido)thiazol-4-yl)-2-((4-(6-ethoxypyrazin-2-yl)phenyl)amino)-2-oxoethyl)carbamate;
2-(2-(cyclopropanesulfonamido)thiazol-4-yl)-2-(dimethylamino)-N-(4-(6-ethoxypyrazin-2-yl)phenyl)acetamide;
2-(2-(cyclopropanesulfonamido)thiazol-4-yl)-N-(4-(6-ethoxypyrazin-2-yl)phenyl)-4-hydroxybutanamide;
2-(2-(cyclopropanesulfonamido)thiazol-4-yl)-N-(4-(6-ethoxypyrazin-2-yl)phenyl)-2-methoxyacetamide;
(R)-2-(2-(cyclopropanesulfonamido)thiazol-4-yl)-N-(4-(6-ethoxypyrazin-2-yl)phenyl)-2-methoxyacetamide;
(S)-2-(2-(cyclopropanesulfonamido)thiazol-4-yl)-N-(4-(6-ethoxypyrazin-2-yl)phenyl)-2-methoxyacetamide;
2-(2-((2-methoxyethyl)sulfonamido)thiazol-4-yl)-2-methyl-N-(5-(6-(trifluoromethyl) pyrazin-2-yl)pyridin-2-yl)propanamide;
2-(2-(cyclopentanesulfonamido)thiazol-4-yl)-2-methyl-N-(4-(pyridin-3-yl)phenyl)propanamide;
2-(2-(cyclopentanesulfonamido)thiazol-4-yl)-2-methyl-N-(4-(6-(trifluoromethyl) pyrazin-2-yl)phenyl)propanamide;
2-(2-(cyclopentanesulfonamido)thiazol-4-yl)-N-(4-(6-ethoxypyrazin-2-yl)phenyl)-2-methylpropanamide;
2-(2-(cyclopropanesulfonamido)thiazol-4-yl)-N-(5-(6-isopropylpyrazin-2-yl)pyridin-2-yl)-2-methylpropanamide;
2-(2-(cyclopropanesulfonamido)thiazol-4-yl)-N-(5′-ethoxy-[3,3′-bipyridin]-6-yl)-2-methylpropanamide;
2-(2-(cyclopropanesulfonamido)thiazol-4-yl)-N-(5-(6-(2-hydroxypropan-2-yl)pyrazin-2-yl)pyridin-2-yl)-2-methylpropanamide;
2-(2-(cyclopropanesulfonamido)thiazol-4-yl)-N-(5-(6-(2-methoxypropan-2-yl)pyrazin-2-yl)pyridin-2-yl)-2-methylpropanamide;
2-(2-(cyclopropanesulfonamido)-5-methylthiazol-4-yl)-2-methyl-N-(5-(6-(trifluoromethyl)pyrazin-2-yl) pyridin-2-yl)propanamide;
2-(2-(cyclopropanesulfonamido)thiazol-4-yl)-N-(5-(6-ethoxypyrazin-2-yl)pyridin-2-yl)-2-methylpropanamide;
2-(2-(cyclopropanesulfonamido)-5-methylthiazol-4-yl)-N-(5-(6-ethoxypyrazin-2-yl) pyridin-2-yl)-2-methylpropanamide;
N-(4-(5-chloropyridin-3-yl)-2-(trifluoromethyl)phenyl)-2-(2-(cyclopropanesulfonamido)thiazol-4-yl)-2-methylpropanamide;
N-(4-(5-cyanopyridin-3-yl)-2-(trifluoromethyl)phenyl)-2-(2-(cyclopropanesulfonamido)thiazol-4-yl)-2-methylpropanamide;
2-(2-(cyclopropanesulfonamido)thiazol-4-yl)-N-(4-(5-fluoropyridin-3-yl)-2-(trifluoromethyl)phenyl)-2-methylpropanamide;
2-(2-(cyclopropanesulfonamido)thiazol-4-yl)-2-methyl-N-(2-(trifluoromethyl)-4-(6-(trifluoromethyl)pyrazin-2-yl)phenyl) propanamide;
2-(2-(cyclopropanesulfonamido)thiazol-4-yl)-N-(4-(6-ethoxypyrazin-2-yl)-2-(trifluoromethyl)phenyl)-2-methylpropanamide;
N-(4-(5-chloropyridin-3-yl)-2,6-diethylphenyl)-2-(2-(cyclopropanesulfonamido)thiazol-4-yl)-2-methylpropanamide;
N-(4-(5-cyanopyridin-3-yl)-2,6-diethylphenyl)-2-(2-(cyclopropanesulfonamido)thiazol-4-yl)-2-methylpropanamide;
2-(2-(cyclopropanesulfonamido)-5-methylthiazol-4-yl)-N-(2-fluoro-4-(5-(trifluoromethyl)pyridin-3-yl)phenyl)-2-methylpropanamide;
N-(4-(5-chloropyridin-3-yl)-2,6-difluorophenyl)-2-(2-(cyclopropanesulfonamido)thiazol-4-yl)-2-methylpropanamide;
N-(4-(5-chloropyridin-3-yl)-2-fluoro-5-methylphenyl)-2-(2-(cyclopropanesulfonamido)thiazol-4-yl)-2-methylpropanamide;
2-(2-(cyclopropanesulfonamido)thiazol-4-yl)-N-(2-fluoro-4-(6-(2-methoxypropan-2-yl)pyrazin-2-yl)phenyl)-2-methylpropanamide;
2-(2-(cyclopropanesulfonamido)-5-methylthiazol-4-yl)-N-(2-fluoro-4-(6-(trifluoromethyl)pyrazin-2-yl)phenyl)-2-methylpropanamide;
N-(4-(6-cyanopyrazin-2-yl)-2-fluorophenyl)-2-(2-(cyclopropanesulfonamido)thiazol-4-yl)-2-methylpropanamide;
2-(2-(cyclopropanesulfonamido)thiazol-4-yl)-N-(4-(6-ethylpyrazin-2-yl)-2-fluorophenyl)-2-methylpropanamide;
2-(2-(cyclopropanesulfonamido)thiazol-4-yl)-N-(2-fluoro-4-(6-isopropoxypyrazin-2-yl)phenyl)-2-methylpropanamide;
2-(2-(cyclopropanesulfonamido)-5-methylthiazol-4-yl)-N-(4-(6-ethoxypyrazin-2-yl)-2-fluorophenyl)-2-methylpropanamide;
N-(4-(5-chloropyridin-3-yl)-2-isopropylphenyl)-2-(2-(cyclopropanesulfonamido)thiazol-4-yl)-2-methylpropanamide;
N-(4-(5-cyanopyridin-3-yl)-2-isopropylphenyl)-2-(2-(cyclopropanesulfonamido)thiazol-4-yl)-2-methylpropanamide;
2-(2-(cyclopropanesulfonamido)thiazol-4-yl)-N-(2-isopropyl-4-(6-(trifluoromethyl)pyrazin-2-yl)phenyl)-2-methylpropanamide;
2-(2-(cyclopropanesulfonamido)thiazol-4-yl)-N-(4-(6-ethoxypyrazin-2-yl)-2-isopropylphenyl)-2-methylpropanamide;
N-(4-(5-chloropyridin-3-yl)-3-fluoro-2-methylphenyl)-2-(2-(cyclopropanesulfonamido)thiazol-4-yl)-2-methylpropanamide;
N-(4-(5-chloropyridin-3-yl)-5-fluoro-2-methylphenyl)-2-(2-(cyclopropanesulfonamido)thiazol-4-yl)-2-methylpropanamide;
N-(4-(5-chloropyridin-3-yl)-2,3-dimethylphenyl)-2-(2-(cyclopropanesulfonamido)thiazol-4-yl)-2-methylpropanamide;
N-(4-(5-chloropyridin-3-yl)-2,5-dimethylphenyl)-2-(2-(cyclopropanesulfonamido)thiazol-4-yl)-2-methylpropanamide;
N-(4-(5-cyanopyridin-3-yl)-3-fluoro-2-methylphenyl)-2-(2-(cyclopropanesulfonamido)thiazol-4-yl)-2-methylpropanamide;
2-(2-(cyclopropanesulfonamido)thiazol-4-yl)-2-methyl-N-(2-methyl-4-(6-(trifluoromethyl)pyrazin-2-yl)phenyl)propanamide;
N-(4-(5-chloropyridin-3-yl)-5-fluoro-2-methoxyphenyl)-2-(2-(cyclopropanesulfonamido)thiazol-4-yl)-2-methylpropanamide;
N-(4-(5-chloropyridin-3-yl)-3-(trifluoromethyl)phenyl)-2-(2-(cyclopropanesulfonamido)thiazol-4-yl)-2-methylpropanamide;
2-(2-(cyclopropanesulfonamido)thiazol-4-yl)-N-(4-(6-ethoxypyrazin-2-yl)-3-methylphenyl)-2-methylpropanamide;
N-(4-(5-chloropyridin-3-yl)-3-ethoxyphenyl)-2-(2-(cyclopropanesulfonamido)thiazol-4-yl)-2-methylpropanamide;
N-(4-(5-chloropyridin-3-yl)phenyl)-1-(2-(cyclopropanesulfonamido)thiazol-4-yl)cyclopropane-1-carboxamide;
N-(4-(5-cyanopyridin-3-yl)phenyl)-2-(2-(cyclopropanesuIfonamido)-5-methylthiazol-4-yl)-2-methylpropanamide;
2-(2-(cyclopropanesulfonamido)thiazol-4-yl)-N-(4-(6-(2-methoxypropan-2-yl)pyrazin-2-yl)phenyl)-2-methylpropanamide;
2-(5-chloro-2-(cyclopropanesulfonamido)thiazol-4-yl)-N-(4-(6-ethoxypyrazin-2-yl)phenyl)-2-methylpropanamide;
2-(2-(cyclopropanesulfonamido)-5-methoxythiazol-4-yl)-N-(4-(6-ethoxypyrazin-2-yl)phenyl)-2-methylpropanamide;
N-(4-(6-(cyclopentylmethoxy)pyrazin-2-yl)phenyl)-2-(2-(cyclopropanesulfonamido)thiazol-4-yl)-2-methylpropanamide;
2-(2-(cyclopropanesulfonamido)thiazol-4-yl)-N-(4-(6-hydroxypyrazin-2-yl)phenyl)-2-methylpropanamide;
2-(2-(ethylsulfonamido)thiazol-4-yl)-2-methyl-N-(5′-(trifluoromethyl)-[3,3′-bipyridin]-6-yl)propanamide;
2-(2-(ethylsulfonamido)thiazol-4-yl)-2-methyl-N-(5-(6-(trifluoromethyl) pyrazin-2-yl) pyridin-2-yl)propanamide;
N-(5-(6-ethoxypyrazin-2-yl)pyridin-2-yl)-2-(2-(ethylsulfonamido)thiazol-4-yl)-2-methylpropanamide;
2-(2-(ethylsulfonamido)thiazol-4-yl)-N-(2-fluoro-4-(6-isopropoxypyrazin-2-yl)phenyl)-2-methylpropanamide;
N-(4-(5-cyanopyridin-3-yl)phenyl)-2-(2-(ethylsulfonamido)thiazol-4-yl)-2-methylpropanamide;
2-(2-(ethylsulfonamido)thiazol-4-yl)-N-(4-(5-fluoropyridin-3-yl)phenyl)-2-methylpropanamide;
2-(2-(ethylsulfonamido)thiazol-4-yl)-2-methyl-N-(4-(pyridin-3-yl)phenyl)propanamide;
2-(2-(ethylsulfonamido)thiazol-4-yl)-2-methyl-N-(4-(6-(trifluoromethyl) pyrazin-2-yl)phenyl)propanamide;
2-(2-(ethylsulfonamido)thiazol-4-yl)-N-(4-(6-isopropoxypyrazin-2-yl)phenyl)-2-methylpropanamide;
2-methyl-2-(2-(methylsulfonamido)thiazol-4-yl)-N-(5′-(trifluoromethyl)-[3,3′-bipyridin]-6-yl)propanamide;
N-(5-(6-ethoxypyrazin-2-yl) pyridin-2-yl)-2-methyl-2-(2-(methylsulfonamido)thiazol-4-yl)propanamide;
N-(2-fluoro-4-(6-(trifluoromethyl) pyrazin-2-yl)phenyl)-2-methyl-2-(2-(methylsulfonamido)thiazol-4-yl)propanamide;
N-(2-fluoro-4-(6-isopropoxypyrazin-2-yl)phenyl)-2-methyl-2-(2-(methylsulfonamido)thiazol-4-yl)propanamide;
N-(4-(5-chloropyridin-3-yl)phenyl)-2-methyl-2-(2-(methylsulfonamido)thiazol-4-yl)propanamide;
2-methyl-2-(2-(methylsulfonamido)thiazol-4-yl)-N-(4-(6-(trifluoromethyl)pyrazin-2-yl)phenyl)propanamide;
N-(4-(6-isopropoxypyrazin-2-yl)phenyl)-2-methyl-2-(2-(methylsulfonamido)thiazol-4-yl)propanamide;
2-(2-((cyclopropylmethyl)sulfonamido)thiazol-4-yl)-2-methyl-N-(5-(6-(trifluoromethyl) pyrazin-2-yl)pyridin-2-yl)propanamide;
1-(2-(cyclopropanesulfonamido)thiazol-4-yl)-N-(4-(6-(trifluoromethyl)pyrazin-2-yl)phenyl)cyclopropane-1-carboxamide;
1-(2-(cyclopropanesulfonamido)thiazol-4-yl)-N-(4-(6-ethoxypyrazin-2-yl)phenyl)cyclopropane-1-carboxamide;
2-(2-(cyclopropanesulfonamido)thiazol-4-yl)-N-(2-fluoro-4-(6-(trifluoromethyl)pyrazin-2-yl)phenyl)-4-methoxybutanamide;
2-(2-(cyclopropanesulfonamido)thiazol-4-yl)-N-(2-fluoro-4-(6-isopropoxypyrazin-2-yl)phenyl)-4-methoxybutanamide;
2-(2-(cyclopropanesulfonamido)thiazol-4-yl)-N-(5-(6-isopropylpyrazin-2-yl)pyridin-2-yl)butanamide;
2-(2-(cyclopropanesulfonamido)thiazol-4-yl)-N-(5-(6-(2-methoxypropan-2-yl)pyrazin-2-yl)pyridin-2-yl)butanamide;
2-(2-(cyclopropanesulfonamido)thiazol-4-yl)-N-(2-fluoro-4-(6-(2-methoxypropan-2-yl)pyrazin-2-yl)phenyl)butanamide;
N-(4-(6-cyanopyrazin-2-yl)-2-fluorophenyl)-2-(2-(cyclopropanesulfonamido)thiazol-4-yl)butanamide;
2-(2-(cyclopropanesulfonamido)thiazol-4-yl)-N-(4-(6-ethylpyrazin-2-yl)-2-fluorophenyl)butanamide;
2-(2-(cyclopropanesulfonamido)thiazol-4-yl)-N-(4-(6-(2-methoxypropan-2-yl)pyrazin-2-yl)phenyl)butanamide;
tert-butyl-(1-(2-(cyclopropanesulfonamido)thiazol-4-yl)-2-((4-(6-ethoxypyrazin-2-yl)phenyl)amino)-2-oxoethyl)carbamate;
2-(2-(cyclopropanesulfonamido)thiazol-4-yl)-N-(2-fluoro-4-(6-(trifluoromethyl)pyrazin-2-yl)phenyl)-2-methoxyacetamide:
2-(2-(cyclopropanesulfonamido)thiazol-4-yl)-N-(4-(6-ethoxypyrazin-2-yl)-2-fluorophenyl)-2-methoxyacetamide;
2-(2-(cyclopropanesulfonamido)thiazol-4-yl)-N-(4-(6-ethoxypyrazin-2-yl)phenyl)-2-methoxyacetamide;
2-(2-(cyclopropanesulfonamido)thiazol-4-yl)-N-(4-(6-isopropoxypyrazin-2-yl)phenyl)-2-methoxyacetamide;
2-(2-(cyclopropanesulfonamido)thiazol-4-yl)-N-(4-(6-ethoxypyrazin-2-yl)-2-fluorophenyl)butanamide;
(R)-2-(2-(cyclopropanesulfonamido)thiazol-4-yl)-N-(4-(6-ethoxypyrazin-2-yl)-2-fluorophenyl)butanamide;
(S)-2-(2-(cyclopropanesulfonamido)thiazol-4-yl)-N-(4-(6-ethoxypyrazin-2-yl)-2-fluorophenyl)butanamide;
2-(2-(cyclopropanesulfonamido)thiazol-4-yl)-N-(5-(6-(trifluoromethyl) pyrazin-2-yl) pyridin-2-yl)butanamide;
2-(2-(cyclopropanesulfonamido)thiazol-4-yl)-N-(2-fluoro-4-(5-(trifluoromethyl)pyridin-3-yl)phenyl)butanamide;
(R)-2-(2-(cyclopropanesulfonamido)thiazol-4-yl)-N-(2-fluoro-4-(5-(trifluoromethyl)pyridin-3-yl)phenyl)butanamide;
(S)-2-(2-(cyclopropanesulfonamido)thiazol-4-yl)-N-(2-fluoro-4-(5-(trifluoromethyl) pyridin-3-yl)phenyl)butanamide;
2-Amino-2-(2-(cyclopropanesuIfonamido)thiazol-4-yl)-N-(5-(6-(trifluoromethyl)pyrazin-2-yl)pyridin-2-yl)acetamide hydrochloride;
2-Amino-2-(2-(cyclopropanesuIfonamido)thiazol-4-yl)-N-(2-fluoro-4-(6-(trifluoromethyl)pyrazin-2-yl)phenyl)acetamide;
2-Amino-2-(2-(cyclopropanesulfonamido)thiazol-4-yl)-N-(4-(6-ethoxypyrazin-2-yl)-2-fluorophenyl)acetamide hydrochloride;
2-(2-(Cyclopropanesulfonamido)thiazol-4-yl)-2-(dimethylamino)-N-(2-fluoro-4-(6-(trifluoromethyl)pyrazin-2-yl)phenyl)acetamide;
2-(2-(Cyclopropanesulfonamido)thiazol-4-yl)-2-(dimethylamino)-N-(4-(6-ethoxypyrazin-2-yl)-2-fluorophenyl)acetamide;
2-(2-(cyclopropanesulfonamido)thiazol-4-yl)-N-(5-(6-ethoxypyrazin-2-yl) pyridin-2-yl)-2,2-difluoroacetamide;
2-(2-(cyclopropanesulfonamido)thiazol-4-yl)-N-(4-(6-ethoxypyrazin-2-yl)-2-fluorophenyl)acetamide;
2-(2-(cyclopropanesulfonamido)thiazol-4-yl)-N-(4-(6-ethoxypyrazin-2-yl)phenyl)acetamide;
2-methyl-2-(2-(methylsulfonamido)thiazol-4-yl)-N-(5-(6-(trifluoromethyl)pyrazin-2-yl) pyridin-2-yl)propanamide;
N-(2-fluoro-4-(5-(trifluoromethyl)pyridin-3-yl)phenyl)-2-methyl-2-(2-(methylsulfonamido)thiazol-4-yl)propanamide;
2-(2-((cyclopropylmethyl)sulfonamido)thiazol-4-yl)-N-(2-fluoro-4-(5-(trifluoromethyl)pyridin-3-yl)phenyl)-2-methylpropanamide;
N-(4-(5-chloro-4-methylpyridin-3-yl)phenyl)-2-(2-(cyclopropanesulfonamido)thiazol-4-yl)-2-methylpropanamide;
N-(4-(6-ethoxypyrazin-2-yl)-2-(trifluoromethyl)phenyl)-2-methyl-2-(2-(methylsulfonamido)thiazol-4-yl)propanamide;
2-(2-((cyclopropylmethyl)sulfonamido)thiazol-4-yl)-N-(2-fluoro-4-(6-(trifluoromethyl)pyrazin-2-yl)phenyl)-2-methylpropanamide;
N-(4-(6-ethoxypyrazin-2-yl)-2-fluorophenyl)-2-methyl-2-(2-(methylsulfonamido)thiazol-4-yl)propanamide;
N-(4-(6-ethoxypyrazin-2-yl)-2-fluorophenyl)-2-(2-((2-methoxyethyl)sulfonamido)thiazol-4-yl)-2-methylpropanamide;
2-(2-((cyclopropylmethyl)sulfonamido)thiazol-4-yl)-N-(4-(6-ethoxypyrazin-2-yl)-2-fluorophenyl)-2-methylpropanamide;
N-(4-(6-ethoxypyrazin-2-yl)phenyl)-2-methyl-2-(2-(methylsulfonamido)thiazol-4-yl)propanamide;
2-(2-(cyclopropanesulfonamido)thiazol-4-yl)-N-(5-(6-ethoxypyrazin-2-yl)-3-fluoropyridin-2-yl)-4-methoxybutanamide;
2-(2-(cyclopropanesulfonamido)thiazol-4-yl)-N-(5-(6-ethoxypyrazin-2-yl)pyridin-2-yl)-4-methoxybutanamide;
2-(2-(cyclopropanesulfonamido)thiazol-4-yl)-4-methoxy-N-(4-(6-(trifluoromethyl)pyrazin-2-yl)phenyl)butanamide;
2-(2-(cyclopropanesulfonamido)thiazol-4-yl)-N-(5-(6-ethoxypyrazin-2-yl)-3-methylpyridin-2-yl)butanamide;
N-(2-chloro-4-(6-ethoxypyrazin-2-yl)phenyl)-2-(2-(cyclopropanesulfonamido)thiazol-4-yl)butanamide;
N-(2-cyano-4-(6-ethoxypyrazin-2-yl)phenyl)-2-(2-(cyclopropanesulfonamido)thiazol-4-yl)butanamide;
2-(2-(cyclopropanesulfonamido)thiazol-4-yl)-N-(4-(6-ethoxypyrazin-2-yl)-2-methylphenyl)butanamide;
2-(2-(cyclopropanesulfonamido)thiazol-4-yl)-N-(4-(6-ethoxypyrazin-2-yl)-2-(trifluoromethoxy)phenyl)butanamide;
2-(2-(cyclopropanesulfonamido)thiazol-4-yl)-N-(4-(6-ethoxypyrazin-2-yl)-2-methoxyphenyl)butanamide;
2-(2-(Cyclopropanesulfonamido)thiazol-4-yl)-N-(4-(6-(ethylamino)pyrazin-2-yl)phenyl)butanamide;
2-(2-(cyclopropanesulfonamido)thiazol-4-yl)-N-(5-(6-ethoxypyrazin-2-yl)-3-fluoropyridin-2-yl)-2-methoxyacetamide;
2-(2-(cyclopropanesulfonamido)thiazol-4-yl)-N-(5-(6-ethoxypyrazin-2-yl)pyridin-2-yl)-2-methoxyacetamide;
2-(2-(cyclopropanesulfonamido)thiazol-4-yl)-N-(4-(5-fluoropyridin-3-yl)-2-(trifluoromethyl)phenyl)-2-methoxyacetamide;
2-(2-(cyclopropanesulfonamido)thiazol-4-yl)-N-(2-fluoro-4-(5-(trifluoromethyl)pyridin-3-yl)phenyl)-2-methoxyacetamide;
2-(2-(cyclopropanesulfonamido)thiazol-4-yl)-N-(4-(6-ethoxypyrazin-2-yl)-2-(trifluoromethyl)phenyl)-2-methoxyacetamide;
N-(2-chloro-4-(6-ethoxypyrazin-2-yl)phenyl)-2-(2-(cyclopropanesulfonamido)thiazol-4-yl)-2-methoxyacetamide;
N-(2-cyano-4-(6-ethoxypyrazin-2-yl)phenyl)-2-(2-(cyclopropanesulfonamido)thiazol-4-yl)-2-methoxyacetamide;
N-(2-fluoro-4-(6-(trifluoromethyl)pyrazin-2-yl)phenyl)-2-methoxy-2-(2-(methylsulfonamido)thiazol-4-yl)acetamide;
2-(2-(cyclopropanesulfonamido)thiazol-4-yl)-N-(4-(6-ethoxypyrazin-2-yl)-2,6-difluorophenyl)-2-methoxyacetamide;
2-(2-(cyclopropanesulfonamido)thiazol-4-yl)-N-(4-(6-ethoxypyrazin-2-yl)-2-(trifluoromethoxy)phenyl)-2-methoxyacetamide;
N-(4-(6-ethoxypyrazin-2-yl)phenyl)-2-methoxy-2-(2-(methylsulfonamido)thiazol-4-yl)acetamide;
2-(2-(cyclopropanesulfonamido)thiazol-4-yl)-N-(5-(6-ethoxypyrazin-2-yl)-3-fluoropyridin-2-yl)butanamide (R enantiomer);
2-(2-(cyclopropanesulfonamido)thiazol-4-yl)-N-(5-(6-ethoxypyrazin-2-yl)-3-fluoropyridin-2-yl)butanamide (S enantiomer);
2-(2-(cyclopropanesulfonamido)thiazol-4-yl)-N-(2-fluoro-4-(6-(trifluoromethyl)pyrazin-2-yl)phenyl)-2-methoxyacetamide (R enantiomer);
2-(2-(cyclopropanesulfonamido)thiazol-4-yl)-N-(2-fluoro-4-(6-(trifluoromethyl)pyrazin-2-yl)phenyl)-2-methoxyacetamide (S enantiomer);
4-(2-(cyclopropanesulfonamido)thiazol-4-yl)-N-(4-(6-ethoxypyrazin-2-yl)-2-fluorophenyl)tetrahydro-2H-pyran-4-carboxamide;
4-(2-(cyclopropanesulfonamido)thiazol-4-yl)-N-(5-(6-(trifluoromethyl)pyrazin-2-yl)pyridin-2-yl)tetrahydro-2H-pyran-4-carboxamide;
4-(2-(cyclopropanesulfonamido)thiazol-4-yl)-N-(5-(6-ethoxypyrazin-2-yl)pyridin-2-yl)tetrahydro-2H-pyran-4-carboxamide:
N-(4-(1-(4-(5-methoxypyridin-3-yl)phenyl)-2-oxopyrrolidin-3-yl)thiazol-2-yl)cyclopropanesulfonamide;
2-(2-(cyclopropanesulfonamido)thiazol-4-yl)-2-methyl-N-(5-(6-methylpyrazin-2-yl) pyridin-2-yl)propanamide; and
N-(4-(6-cyanopyrazin-2-yl)-2-methylphenyl)-2-(2-(cyclopropanesulfonamido)thiazol-4-yl)-2-methylpropanamide;
   or a pharmaceutically acceptable salt and/or pharmaceutically acceptable solvate thereof.

Such CTPS1 inhibitors are disclosed in PCT publication number WO2019106156, which is incorporated by reference in its entirety for the purpose of the CTPS1 inhibitors disclosed therein. In particular a CTPS1 inhibitor may be a compound described in any one of clauses 1 to 118 of WO201 9106156, or a pharmaceutically acceptable salt and/or pharmaceutically acceptable solvate thereof, in particular a compound T1 to T465 or a pharmaceutically acceptable salt and/or pharmaceutically acceptable solvate thereof.

Alternatively, the CTPS1 inhibitor is a compound formula (III):

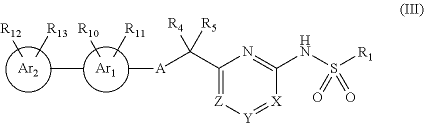

wherein
A is an amide linker having the following structure: —C(═O)NH— or —NHC(═O)—;
X is N or CH;
Y is N or CR₂;
Z is N or CR₃,
   with the proviso that when at least one of X or Z is N, Y cannot be N;
R₁ is C₁₋₅alkyl, C₀₋₂alkyleneC₃₋₅cycloalkyl which cycloalkyl is optionally substituted by
CH₃, or CF₃;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
   wherein at least one of R₂ and R₃ is H;
R₄ and R₅ are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R₄ and R₅ together with the carbon atom to which they are attached form a C₃₋₆cycloalkyl or C₃₋₆heterocycloalkyl; and
   when A is —NHC(═O)—;
   R₄ and R₅ may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and
   NR21 R22:
Ar1 is a 6-membered aryl or heteroaryl;
Ar2 is a 6-membered aryl or heteroaryl and is attached to Ar1 in the para position relative to the amide;
R₁₀ is H, halo, C₁₋₃alkyl, C₁₋₂haloalkyl, OC₁₋₂alkyl, OC₁₋₂haloalkyl or CN;
R₁₁ is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₀₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
   when A is —NHC(═O)—;
   R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N^{+—O—);}
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;

or a pharmaceutically acceptable salt and/or pharmaceutically acceptable solvate thereof.

More suitably, the CTPS1 inhibitor is selected from the following (‘List C’) compounds:
N-(4-(5-chloropyridin-3-yl)phenyl)-2-(2-(cyclopropanesulfonamido)pyrimidin-4-yl)butanamide;
1-(2-(cyclopropanesulfonamido)pyrimidin-4-yl)-N-(4-(6-ethoxypyrazin-2-yl)phenyl)cyclopentanecarboxamide;
2-(2-(cyclopropanesulfonamido)pyrimidin-4-yl)-N-(4-(6-methoxypyrazin-2-yl)phenyl)-2-methylpropanamide;
2-(2-(cyclopropanesulfonamido)pyrimidin-4-yl)-2-methyl-N-(4-(5-(trifluoromethyl)pyridin-3-yl)phenyl)propanamide;
2-methyl-N-(2-methyl-4-(6-methylpyrazin-2-yl)phenyl)-2-(2-(methylsulfonamido)pyrimidin-4-yl)propanamide;
2-(2-(cyclopropanesulfonamido)pyrimidin-4-yl)-N-(2-fluoro-4-(pyrazin-2-yl)phenyl)butanamide;
2-(2-(cyclopropanesulfonamido)pyrimidin-4-yl)-N-(4-(5-(trifluoromethyl)pyridin-3-yl)phenyl)butanamide;
2-(2-(cyclopropanesulfonamido)pyrimidin-4-yl)-N-(4-(6-(trifluoromethyl)pyrazin-2-yl)phenyl)acetamide;
2-(2-(cyclopropanesulfonamido)pyrimidin-4-yl)-N-(5-(6-isopropoxypyrazin-2-yl)pyridin-2-yl)-2-methylpropanamide;
2-(2-(cyclopropanesulfonamido)pyrimidin-4-yl)-N-(4-(6-ethoxypyrazin-2-yl)phenyl)-2-ethylbutanamide;
2-(2-(cyclopropanesulfonamido)pyrimidin-4-yl)-N-(2-fluoro-4-(6-(trifluoromethyl) pyrazin-2-yl)phenyl)acetamide;
2-(2-(cyclopropanesulfonamido)pyrimidin-4-yl)-N-(2-fluoro-4-(6-isopropoxypyrazin-2-yl)phenyl)acetamide;
2-(2-(cyclopropanesulfonamido)pyrimidin-4-yl)-N-(4-(5-(trifluoromethyl)pyridin-3-yl)phenyl)acetamide;
2-(2-(cyclopropanesulfonamido)pyrimidin-4-yl)-N-(4-(5-(2,2,2-trifluoroethoxy)pyridin-3-yl)phenyl)acetamide;
2-(2-(cyclopropanesulfonamido)-5-fluoropyrimidin-4-yl)-N-(4-(pyridin-3-yl)phenyl)acetamide;
2-(2-(cyclopropanesulfonamido)pyrimidin-4-yl)-N-(4-(pyridin-3-yl)phenyl)acetamide;
N-([1,1′-biphenyl]-4-yl)-2-(2-(cyclopropanesulfonamido)pyrimidin-4-yl)acetamide;
2-(2-(cyclopropanesulfonamido)pyrimidin-4-yl)-N-(4-(6-ethoxypyrazin-2-yl)phenyl)acetamide;
2-(2-(cyclopropanesulfonamido)pyrimidin-4-yl)-N-(4-(6-methoxypyrazin-2-yl)phenyl)acetamide;
2-(2-(cyclopropanesulfonamido)pyrimidin-4-yl)-N-(4-(6-(2,2,2-trifluoroethoxy)pyrazin-2-yl)phenyl)acetamide;
2-(2-(cyclopropanesulfonamido)pyrimidin-4-yl)-N-(4-(6-isopropoxypyrazin-2-yl)phenyl)acetamide;
2-(2-(cyclobutanesulfonamido)pyrimidin-4-yl)-N-(5-(6-ethoxypyrazin-2-yl)pyridin-2-yl)-2-methylpropanamide;
2-(2-(cyclobutanesulfonamido)pyrimidin-4-yl)-N-(2-fluoro-4-(6-isopropoxypyrazin-2-yl)phenyl)-2-methylpropanamide;
2-(2-(cyclobutanesulfonamido)pyrimidin-4-yl)-N-(4-(6-ethoxypyrazin-2-yl)-2-methylphenyl)-2-methylpropanamide;
2-(2-(cyclobutanesulfonamido)pyrimidin-4-yl)-N-(4-(6-methoxypyrazin-2-yl)phenyl)-2-methylpropanamide;
2-(2-(cyclobutanesulfonamido)pyrimidin-4-yl)-N-(4-(6-ethoxypyrazin-2-yl)phenyl)-2-methylpropanamide;
2-(2-(cyclopropanesulfonamido)pyrimidin-4-yl)-N-(5-(6-ethoxypyrazin-2-yl)-3-fluoropyridin-2-yl)-2-methylpropanamide;
2-(2-(cyclopropanesulfonamido)pyrimidin-4-yl)-N-(5′-ethoxy-[3,3′-bipyridin]-6-yl)-2-methylpropanamide;
N-([3,3′-bipyridin]-6-yl)-2-(2-(cyclopropanesulfonamido)pyrimidin-4-yl)-2-methylpropanamide;
2-(2-(cyclopropanesulfonamido)pyrimidin-4-yl)-2-methyl-N-(5-(6-(trifluoromethyl)pyrazin-2-yl)pyridin-2-yl)propanamide;
2-(2-(cyclopropanesulfonamido)pyrimidin-4-yl)-N-(5-(6-ethoxypyrazin-2-yl)pyridin-2-yl)-2-methylpropanamide;
2-(2-(cyclopropanesulfonamido)pyrimidin-4-yl)-N-(5-(6-cyclopropoxypyrazin-2-yl) pyridin-2-yl)-2-methylpropanamide;
N-(2-chloro-4-(6-ethoxypyrazin-2-yl)phenyl)-2-(2-(cyclopropanesulfonamido)pyrimidin-4-yl)-2-methylpropanamide;
N-(2-cyano-4-(6-ethoxypyrazin-2-yl)phenyl)-2-(2-(cyclopropanesulfonamido)pyrimidin-4-yl)-2-methylpropanamide;
2-(2-(cyclopropanesulfonamido)pyrimidin-4-yl)-N-(2-fluoro-4-(5-isopropoxypyridin-3-yl)phenyl)-2-methylpropanamide;
2-(2-(cyclopropanesulfonamido)pyrimidin-4-yl)-N-(2-fluoro-4-(pyridin-3-yl)phenyl)-2-methylpropanamide;
2-(2-(cyclopropanesulfonamido)pyrimidin-4-yl)-N-(2-fluoro-4-(6-(trifluoromethyl) pyrazin-2-yl)phenyl)-2-methylpropanamide;
2-(2-(cyclopropanesulfonamido)pyrimidin-4-yl)-N-(4-(6-ethoxypyrazin-2-yl)-2-fluorophenyl)-2-methylpropanamide;
2-(2-(cyclopropanesulfonamido)pyrimidin-4-yl)-N-(2-fluoro-4-(6-isopropoxypyrazin-2-yl)phenyl)-2-methylpropanamide;
2-(2-(cyclopropanesulfonamido)pyrimidin-4-yl)-N-(4-(6-ethoxypyrazin-2-yl)-2-fluoro-5-methylphenyl)-2-methylpropanamide;
2-(2-(cyclopropanesulfonamido)pyrimidin-4-yl)-N-(4-(6-ethoxypyrazin-2-yl)-2,6-difluorophenyl)-2-methylpropanamide;
2-(2-(cyclopropanesulfonamido)pyrimidin-4-yl)-N-(2-fluoro-4-(pyrazin-2-yl)phenyl)-2-methylpropanamide;
2-(2-(cyclopropanesulfonamido)pyrimidin-4-yl)-2-methyl-N-(2-methyl-4-(6-(trifluoromethyl)pyrazin-2-yl)phenyl) propanamide;
2-(2-(cyclopropanesulfonamido)pyrimidin-4-yl)-N-(4-(6-ethoxypyrazin-2-yl)-2,3-dimethylphenyl)-2-methylpropanamide;
2-(2-(cyclopropanesulfonamido)pyrimidin-4-yl)-N-(4-(6-ethoxypyrazin-2-yl)-5-fluoro-2-methylphenyl)-2-methylpropanamide;
2-(2-(cyclopropanesulfonamido)pyrimidin-4-yl)-N-(4-(6-ethoxypyrazin-2-yl)-2,5-dimethylphenyl)-2-methylpropanamide;
2-(2-(cyclopropanesulfonamido)pyrimidin-4-yl)-N-(4-(6-ethoxypyrazin-2-yl)-2-(trifluoromethoxy)phenyl)-2-methylpropanamide;
2-(2-(cyclopropanesulfonamido)pyrimidin-4-yl)-N-(4-(6-ethoxypyrazin-2-yl)-5-fluoro-2-methoxyphenyl)-2-methylpropanamide;
2-(2-(cyclopropanesulfonamido)pyrimidin-4-yl)-N-(4-(6-ethoxypyrazin-2-yl)-2-methoxyphenyl)-2-methylpropanamide;
2-(2-(cyclopropanesulfonamido)pyrimidin-4-yl)-2-methyl-N-(4-(pyrimidin-5-yl)phenyl)propanamide;
N-(4-(5-chloropyridin-3-yl)phenyl)-2-(2-(cyclopropanesulfonamido)pyrimidin-4-yl)-2-methylpropanamide;
N-(4-(5-cyanopyridin-3-yl)phenyl)-2-(2-(cyclopropanesuIfonamido)pyrimidin-4-yl)-2-methylpropanamide;
2-(2-(cyclopropanesulfonamido)pyrimidin-4-yl)-N-(4-(5-fluoropyridin-3-yl)phenyl)-2-methylpropanamide;
2-(2-(cyclopropanesulfonamido)pyrimidin-4-yl)-2-methyl-N-(4-(5-methylpyridin-3-yl)phenyl)propanamide;
2-(2-(cyclopropanesulfonamido)pyrimidin-4-yl)-N-(4-(5-(difluoromethoxy)pyridin-3-yl)phenyl)-2-methylpropanamide;
2-(2-(cyclopropanesulfonamido)pyrimidin-4-yl)-N-(4-(5-methoxypyridin-3-yl)phenyl)-2-methylpropanamide;
2-(2-(cyclopropanesulfonamido)pyrimidin-4-yl)-N-(4-(5-ethoxypyridin-3-yl)phenyl)-2-methylpropanamide;
2-(2-(cyclopropanesulfonamido)pyrimidin-4-yl)-N-(4-(5-isopropoxypyridin-3-yl)phenyl)-2-methylpropanamide;
2-(2-(cyclopropanesulfonamido)pyrimidin-4-yl)-2-methyl-N-(4-(pyridin-3-yl)phenyl)propanamide;
2-(2-(cyclopropanesulfonamido)pyrimidin-4-yl)-2-methyl-N-(3′-(trifluoromethyl)-[1,1′-biphenyl]-4-yl)propanamide;
N-(3′-chloro-[1,1′-biphenyl]-4-yl)-2-(2-(cyclopropanesulfonamido)pyrimidin-4-yl)-2-methylpropanamide;
N-(3′-cyano-[1,1′-biphenyl]-4-yl)-2-(2-(cyclopropanesulfonamido)pyrimidin-4-yl)-2-methylpropanamide;
2-(2-(cyclopropanesulfonamido)pyrimidin-4-yl)-N-(3′-ethoxy-[1,1′-biphenyl]-4-yl)-2-methylpropanamide;
2-(2-(cyclopropanesulfonamido)pyrimidin-4-yl)-2-methyl-N-(4-(6-(trifluoromethyl)pyrazin-2-yl)phenyl)propanamide;
2-(2-(cyclopropanesulfonamido)pyrimidin-4-yl)-N-(4-(6-ethoxypyrazin-2-yl)phenyl)-2-methylpropanamide;
2-(2-(cyclopropanesulfonamido)pyrimidin-4-yl)-N-(4-(6-cyclopropoxypyrazin-2-yl)phenyl)-2-methylpropanamide;
2-(2-(cyclopropanesulfonamido)pyrimidin-4-yl)-N-(4-(6-isopropoxypyrazin-2-yl)phenyl)-2-methylpropanamide;
2-(2-(cyclopropanesulfonamido)-5-fluoropyrimidin-4-yl)-N-(4-(6-ethoxypyrazin-2-yl)phenyl)-2-methylpropanamide;
N-(4-(6-ethoxypyrazin-2-yl)phenyl)-2-methyl-2-(2-((1-methylcyclopropane)-1-sulfonamido)pyrimidin-4-yl)propanamide;
2-(2-(cyclopropanesulfonamido)-5-methylpyrimidin-4-yl)-N-(4-(6-ethoxypyrazin-2-yl)phenyl)-2-methylpropanamide;
2-(2-(cyclopropanesulfonamido)pyrimidin-4-yl)-2-methyl-N-(4-(pyrazin-2-yl)phenyl)propanamide;
N-(4-(6-ethoxypyrazin-2-yl)-2-fluorophenyl)-2-(2-(ethylsulfonamido)pyrimidin-4-yl)-2-methylpropanamide;
2-(2-(ethylsulfonamido)pyrimidin-4-yl)-2-methyl-N-(4-(6-(trifluoromethyl)pyrazin-2-yl)phenyl)propanamide;
N-(4-(6-ethoxypyrazin-2-yl)phenyl)-2-(2-(ethylsulfonamido)pyrimidin-4-yl)-2-methylpropanamide;
N-(5-(6-ethoxypyrazin-2-yl)-3-fluoropyridin-2-yl)-2-methyl-2-(2-(methylsulfonamido)pyrimidin-4-yl)propanamide;
N-(5-(6-ethoxypyrazin-2-yl)pyridin-2-yl)-2-methyl-2-(2-(methylsulfonamido)pyrimidin-4-yl)propanamide;
N-(2-fluoro-4-(5-isopropoxypyridin-3-yl)phenyl)-2-methyl-2-(2-(methylsulfonamido)pyrimidin-4-yl)propanamide;
N-(2-fluoro-4-(6-isopropoxypyrazin-2-yl)phenyl)-2-methyl-2-(2-(methylsulfonamido)pyrimidin-4-yl)propanamide;
2-methyl-N-(2-methyl-4-(6-(trifluoromethyl)pyrazin-2-yl)phenyl)-2-(2-(methylsulfonamido)pyrimidin-4-yl)propanamide;
2-methyl-2-(2-(methylsulfonamido)pyrimidin-4-yl)-N-(4-(6-(trifluoromethyl)pyrazin-2-yl)phenyl)propanamide;
N-(4-(6-ethoxypyrazin-2-yl)phenyl)-2-methyl-2-(2-(methylsulfonamido)pyrimidin-4-yl)propanamide;
2-(2-((1,1-dimethylethyl)sulfonamido)pyrimidin-4-yl)-N-(4-(6-ethoxypyrazin-2-yl)phenyl)-2-methylpropanamide;
1-(2-(cyclopropanesulfonamido)pyrimidin-4-yl)-N-(4-(6-ethoxypyrazin-2-yl)phenyl)cyclopropanecarboxamide;
2-(2-(cyclopropanesulfonamido)pyrimidin-4-yl)-N-(5′-(trifluoromethyl)-[3,3′-bipyridin]-6-yl)butanamide;
2-(2-(cyclopropanesulfonamido)pyrimidin-4-yl)-N-(5′-(2,2,2-trifluoroethoxy)-[3,3′-bipyridin]-6-yl)butanamide;
N-([3,3′-bipyridin]-6-yl)-2-(2-(cyclopropanesulfonamido)pyrimidin-4-yl)butanamide;
2-(2-(cyclopropanesulfonamido)pyrimidin-4-yl)-N-(5-(6-(trifluoromethyl)pyrazin-2-yl)pyridin-2-yl)butanamide;
2-(2-(cyclopropanesulfonamido)pyrimidin-4-yl)-N-(5-(6-ethoxypyrazin-2-yl)pyridin-2-yl)butanamide;
2-(2-(cyclopropanesulfonamido)pyrimidin-4-yl)-N-(5-(6-isopropoxypyrazin-2-yl)pyridin-2-yl)butanamide;
N-(4-(5-chloropyridin-3-yl)-2-fluorophenyl)-2-(2-(cyclopropanesulfonamido)pyrimidin-4-yl)butanamide;
2-(2-(cyclopropanesulfonamido)pyrimidin-4-yl)-N-(2-fluoro-4-(5-(2,2,2-trifluoroethoxy)pyridin-3-yl)phenyl)butanamide;
2-(2-(cyclopropanesulfonamido)pyrimidin-4-yl)-N-(2-fluoro-4-(5-isopropoxypyridin-3-yl)phenyl)butanamide;
2-(2-(cyclopropanesulfonamido)pyrimidin-4-yl)-N-(2-fluoro-4-(pyridin-3-yl)phenyl)butanamide;
2-(2-(cyclopropanesulfonamido)pyrimidin-4-yl)-N-(2-fluoro-4-(6-(trifluoromethyl)pyrazin-2-yl)phenyl)butanamide;
2-(2-(cyclopropanesulfonamido)pyrimidin-4-yl)-N-(2-fluoro-4-(6-methoxypyrazin-2-yl)phenyl)butanamide;
2-(2-(cyclopropanesulfonamido)pyrimidin-4-yl)-N-(4-(6-ethoxypyrazin-2-yl)-2-fluorophenyl)butanamide;
2-(2-(cyclopropanesulfonamido)pyrimidin-4-yl)-N-(2-fluoro-4-(6-isopropoxypyrazin-2-yl)phenyl)butanamide;
2-(2-(cyclopropanesulfonamido)pyrimidin-4-yl)-N-(2-fluoro-4-(6-(2,2,2-trifluoroethoxy)pyrazin-2-yl)phenyl)butanamide;
N-(4-(5-cyanopyridin-3-yl)phenyl)-2-(2-(cyclopropanesulfonamido)pyrimidin-4-yl)butanamide;
2-(2-(cyclopropanesulfonamido)pyrimidin-4-yl)-N-(4-(5-(2,2,2-trifluoroethoxy)pyridin-3-yl)phenyl)butanamide;
2-(2-(cyclopropanesulfonamido)pyrimidin-4-yl)-N-(4-(5-isopropoxypyridin-3-yl)phenyl)butanamide;
2-(2-(cyclopropanesulfonamido)pyrimidin-4-yl)-N-(4-(pyridin-3-yl)phenyl)butanamide;
2-(2-(cyclopropanesulfonamido)pyrimidin-4-yl)-N-(4-(6-(trifluoromethyl)pyrazin-2-yl)phenyl)butanamide;
N-(4-(6-chloropyrazin-2-yl)phenyl)-2-(2-(cyclopropanesulfonamido)pyrimidin-4-yl)butanamide;
2-(2-(cyclopropanesulfonamido)pyrimidin-4-yl)-N-(4-(6-ethoxypyrazin-2-yl)phenyl)butanamide;
2-(2-(cyclopropanesulfonamido)pyrimidin-4-yl)-N-(4-(6-methoxypyrazin-2-yl)phenyl)butanamide;
2-(2-(cyclopropanesulfonamido)pyrimidin-4-yl)-N-(4-(6-isopropoxypyrazin-2-yl)phenyl)butanamide;
2-(2-(cyclopropanesulfonamido)pyrimidin-4-yl)-N-(4-(6-(2,2,2-trifluoroethoxy)pyrazin-2-yl)phenyl)butanamide;
2-(2-(cyclopropanesulfonamido)pyrimidin-4-yl)-N-(4-(pyrazin-2-yl)phenyl)butanamide;
2-(2-(cyclopropanesulfonamido)pyrimidin-4-yl)-N-(4-(6-ethoxypyrazin-2-yl)phenyl)-4-methoxybutanamide;
2-(2-(cyclopropanesulfonamido)pyrimidin-4-yl)-N-(4-(pyridin-3-yl)phenyl)propenamide;
2-(2-(cyclopropanesulfonamido)pyrimidin-4-yl)-N-(5-(6-ethoxypyrazin-2-yl)pyridin-2-yl)-2-(R)-fluorobutanamide;
2-(2-(cyclopropanesulfonamido)pyrimidin-4-yl)-N-(5-(6-ethoxypyrazin-2-yl)pyridin-2-yl)-2-(S)-fluorobutanamide;
2-(2-(cyclopropanesulfonamido)pyrimidin-4-yl)-N-(5-(6-ethoxypyrazin-2-yl)pyridin-2-yl)-2-fluorobutanamide;
4-(2-(cyclopropanesulfonamido)pyrimidin-4-yl)-N-(5-(6-ethoxypyrazin-2-yl)pyridin-2-yl)tetrahydro-2H-pyran-4-carboxamide;
2-(2-(cyclopropanesulfonamido)pyrimidin-4-yl)-N-(5-(6-isopropylpyrazin-2-yl) pyridin-2-yl)-2-methylpropanamide;
2-(2-(cyclopropanesulfonamido)pyrimidin-4-yl)-N-(4-(6-ethoxypyrazin-2-yl)-2-fluorophenyl)-2,2-difluoroacetamide;
N-((2-(cyclopropanesulfonamido)pyrimidin-4-yl)methyl)-4-(6-ethoxypyrazin-2-yl)benzamide;
2-(2-(cyclopropanesulfonamido)pyrimidin-4-yl)-2-methyl-N-(5-(6-(prop-1-en-2-yl)pyrazin-2-yl)pyridin-2-yl)propanamide;
2-(2-(cyclopropanesulfonamido)-6-methylpyrimidin-4-yl)-N-(5-(6-ethoxypyrazin-2-yl)pyridin-2-yl)-2-methylpropanamide;
2-(2-(cyclopropanesulfonamido)-6-(trifluoromethyl)pyrimidin-4-yl)-N-(5-(6-ethoxypyrazin-2-yl)pyridin-2-yl)-2-methylpropanamide;
2-(2-(cyclopropanesulfonamido)pyrimidin-4-yl)-N-(5-(6-cyclopropylpyrazin-2-yl)pyridin-2-yl)-2-methylpropanamide;
2-(2-(cyclopropanesulfonamido)pyrimidin-4-yl)-N-(6-(6-ethoxypyrazin-2-yl)pyridin-3-yl)-2-methylpropanamide;
2-(2-(cyclopropanesulfonamido)pyrimidin-4-yl)-N-(4-(6-cyclopropylpyrazin-2-yl)-2-fluorophenyl)-2-methylpropanamide;
2-(2-(cyclopropanesulfonamido)-6-methylpyrimidin-4-yl)-N-(4-(6-ethoxypyrazin-2-yl)-2-fluorophenyl)-2-methylpropanamide;
2-(2-(cyclopropanesulfonamido)-6-(trifluoromethyl)pyrimidin-4-yl)-N-(4-(6-ethoxypyrazin-2-yl)-2-fluorophenyl)-2-methylpropanamide;
2-(2-(cyclopropanesulfonamido)pyrimidin-4-yl)-2-methyl-N-(4-(6-(prop-1-en-2-yl)pyrazin-2-yl)phenyl)propanamide;
2-(2-(cyclopropanesulfonamido)pyrimidin-4-yl)-N-(4-(6-isopropylpyrazin-2-yl)phenyl)-2-methylpropanamide;
2-(2-(cyclopropanesulfonamido)pyrimidin-4-yl)-N-(4-(6-(dimethylamino)pyrazin-2-yl)phenyl)-2-methylpropanamide;
2-(2-(cyclopropanesulfonamido)-6-methylpyrimidin-4-yl)-N-(4-(6-ethoxypyrazin-2-yl)phenyl)-2-methylpropanamide;
2-(2-(cyclopropanesulfonamido)-6-(trifluoromethyl)pyrimidin-4-yl)-N-(4-(6-ethoxypyrazin-2-yl)phenyl)-2-methylpropanamide;
2-(2-(cyclopropanesulfonamido)-6-methoxypyrimidin-4-yl)-2-methyl-N-(4-(pyridin-3-yl)phenyl)propanamide;
1-(2-(cyclopropanesulfonamido)pyrimidin-4-yl)-N-(5-(6-ethoxypyrazin-2-yl)pyridin-2-yl)cyclopentane-1-carboxamide;
4-(2-(cyclopropanesulfonamido)pyrimidin-4-yl)-N-(4-(6-ethoxypyrazin-2-yl)phenyl)tetrahydro-2H-pyran-4-carboxamide;
N-(5-(6-ethoxypyrazin-2-yl)pyridin-2-yl)-4-(2-(methylsulfonamido)pyrimidin-4-yl)piperidine-4-carboxamide;
tert-butyl 4-(2-(cyclopropanesulfonamido)pyrimidin-4-yl)-4-((5-(6-ethoxypyrazin-2-yl)pyridin-2-yl)carbamoyl)piperidine-1-carboxylate;
4-(2-(cyclopropanesulfonamido)pyrimidin-4-yl)-N-(5-(6-ethoxypyrazin-2-yl)pyridin-2-yl)piperidine-4-carboxamide;
tert-butyl 3-(2-(cyclopropanesulfonamido)pyrimidin-4-yl)-3-((5-(6-ethoxypyrazin-2-yl)pyridin-2-yl)carbamoyl)azetidine-1-carboxylate;
tert-butyl 4-((5-(6-ethoxypyrazin-2-yl) pyridin-2-yl)carbamoyl)-4-(2-(methylsulfonamido)pyrimidin-4-yl)piperidine-1-carboxylate;
4-(2-(cyclopropanesulfonamido)pyrimidin-4-yl)-N-(4-(6-ethoxypyrazin-2-yl)-2-fluorophenyl)tetrahydro-2H-pyran-4-carboxamide;
2-(2-(cyclopropanesulfonamido)pyrimidin-4-yl)-N-(5-(6-ethoxypyrazin-2-yl)-3-fluoropyridin-2-yl)-4-methoxybutanamide;
2-(2-(cyclopropanesulfonamido)pyrimidin-4-yl)-N-(5-(6-ethoxypyrazin-2-yl)pyridin-2-yl)-4-methoxybutanamide;
2-(2-(cyclopropanesulfonamido)pyrimidin-4-yl)-N-(4-(6-ethoxypyrazin-2-yl)-2-fluorophenyl)-4-methoxybutanamide;
N-(5-(6-ethoxypyrazin-2-yl)pyridin-2-yl)-4-methoxy-2-methyl-2-(2-(methylsulfonamido)pyrimidin-4-yl)butanamide;
N-(5′-chloro-[3,3′-bipyridin]-6-yl)-2-(2-(cyclopropanesulfonamido)pyrimidin-4-yl)butanamide;
N-(5′-chloro-[3,3′-bipyridin]-6-yl)-2-(2-(cyclopropanesulfonamido)pyrimidin-4-yl)-2-fluorobutanamide;
2-(2-(cyclopropanesulfonamido)pyrimidin-4-yl)-N-(5-(6-cyclopropylpyrazin-2-yl)pyridin-2-yl)-2-fluorobutanamide;
N-(5-(6-ethoxypyrazin-2-yl)pyridin-2-yl)-2-fluoro-2-(2-(methylsulfonamido)pyrimidin-4-yl)butanamide;
2-(2-(cyclopropanesulfonamido)pyrimidin-4-yl)-N-(5-(6-ethoxypyrazin-2-yl)-3-methylpyridin-2-yl)butanamide;
2-(2-(cyclopropanesulfonamido)pyrimidin-4-yl)-N-(5-(6-cyclopropylpyrazin-2-yl)pyridin-2-yl)butanamide;
2-(2-(cyclopropanesulfonamido)pyrimidin-4-yl)-N-(5-(6-(2,2,2-trifluoroethoxy)pyrazin-2-yl)pyridin-2-yl)butanamide;
2-(2-(cyclopropanesulfonamido)pyrimidin-4-yl)-N-(3-fluoro-5-(6-methoxypyrazin-2-yl)pyridin-2-yl)butanamide;
2-(2-(cyclopropanesulfonamido)pyrimidin-4-yl)-N-(5-(6-methoxypyrazin-2-yl)pyridin-2-yl)butanamide;
2-(2-(cyclopropanesulfonamido)pyrimidin-4-yl)-N-(4-(6-cyclopropylpyrazin-2-yl)-2-fluorophenyl)butanamide;
2-(2-(cyclopropanesulfonamido)pyrimidin-4-yl)-N-(4-(6-ethoxypyrazin-2-yl)-2-methylphenyl)butanamide;
2-(2-(cyclopropanesulfonamido)pyrimidin-4-yl)-N-(5-(6-ethoxypyrazin-2-yl)-3-fluoropyridin-2-yl)butanamide;
2-(2-(cyclopropanesulfonamido)pyrimidin-4-yl)-N-(5-(6-ethoxypyrazin-2-yl)pyridin-2-yl)-2-methylbutanamide;
2-(2-(cyclopropanesulfonamido)pyrimidin-4-yl)-N-(5-(6-ethoxypyrazin-2-yl)pyridin-2-yl)-2-fluoro-3-methylbutanamide;
2-(2-(cyclopropanesulfonamido)pyrimidin-4-yl)-N-(4-(6-ethoxypyrazin-2-yl)-2-fluorophenyl)-3-methylbutanamide;
2-(2-(cyclopropanesulfonamido)pyrimidin-4-yl)-N-(5-(6-ethoxypyrazin-2-yl)pyridin-2-yl)-3-methylbutanamide;
2-(2-(cyclopropanesulfonamido)pyrimidin-4-yl)-N-(4-(6-ethoxypyrazin-2-yl)phenyl)-2-methoxyacetamide;
N-(5-(6-ethoxypyrazin-2-yl)pyridin-2-yl)-2-fluoro-2-(2-(methylsulfonamido)pyrimidin-4-yl)-(R)-butanamide:
N-(5-(6-ethoxypyrazin-2-yl) pyridin-2-yl)-2-fluoro-2-(2-(methylsulfonamido)pyrimidin-4-yl)-(S)-butanamide;
N-(4-(5-chloropyridin-3-yl)phenyl)-2-(6-(cyclopropanesulfonamido)pyridin-2-yl)acetamide;
N-(4-(5-cyanopyridin-3-yl)phenyl)-2-(6-(cyclopropanesulfonamido)pyridin-2-yl)acetamide;
2-(6-(cyclopropanesulfonamido)pyridin-2-yl)-N-(4-(5-fluoropyridin-3-yl)phenyl)acetamide;
2-(6-(cyclopropanesulfonamido)pyridin-2-yl)-N-(4-(5-methoxypyridin-3-yl)phenyl)acetamide;
2-(6-(cyclopropanesulfonamido)pyridin-2-yl)-N-(4-(pyridin-3-yl)phenyl)acetamide;
2-(6-(cyclopropanesulfonamido)pyridin-2-yl)-N-(4-(6-(trifluoromethyl)pyrazin-2-yl)phenyl)acetamide;
2-(6-(cyclopropanesulfonamido)pyridin-2-yl)-N-(4-(6-methoxypyrazin-2-yl)phenyl)acetamide;
2-(6-(cyclopropanesulfonamido)pyridin-2-yl)-N-(4-(pyrazin-2-yl)phenyl)acetamide;
N-([3,3′-bipyridin]-6-yl)-2-(6-(cyclopropanesulfonamido)pyridin-2-yl)-2-methylpropanamide;
N-(4-(5-chloropyridin-3-yl)phenyl)-2-(6-(cyclopropanesulfonamido)pyridin-2-yl)-2-methylpropanamide;
2-(6-(cyclopropanesulfonamido)pyridin-2-yl)-N-(4-(5-fluoropyridin-3-yl)phenyl)-2-methylpropanamide;
2-(6-(cyclopropanesulfonamido)pyridin-2-yl)-N-(4-(5-ethoxypyridin-3-yl)phenyl)-2-methylpropanamide;
2-(6-(cyclopropanesulfonamido)pyridin-2-yl)-2-methyl-N-(4-(pyridin-3-yl)phenyl) propanamide;
2-(6-(cyclopropanesulfonamido)pyridin-2-yl)-N-(2-fluoro-4-(pyrazin-2-yl)phenyl)-2-methylpropanamide;
2-(6-(cyclopropanesulfonamido)pyridin-2-yl)-2-methyl-N-(4-(6-(trifluoromethyl)pyrazin-2-yl)phenyl)propanamide;
N-(4-(6-chloropyrazin-2-yl)phenyl)-2-(6-(cyclopropanesulfonamido)pyridin-2-yl)-2-methylpropanamide;
2-(6-(cyclopropanesulfonamido)pyridin-2-yl)-N-(4-(6-ethoxypyrazin-2-yl)phenyl)-2-methylpropanamide;
2-(6-(cyclopropanesulfonamido)pyridin-2-yl)-N-(4-(6-methoxypyrazin-2-yl)phenyl)-2-methylpropanamide;
2-(6-(cyclopropanesulfonamido)pyridin-2-yl)-2-methyl-N-(4-(pyrazin-2-yl)phenyl)propanamide;
4-(6-(cyclopropanesulfonamido)pyridin-2-yl)-N-(5-(6-ethoxypyrazin-2-yl)pyridin-2-yl)tetrahydro-2H-pyran-4-carboxamide;
2-(6-(cyclopropanesulfonamido)pyridin-2-yl)-N-(5-(6-(trifluoromethyl)pyrazin-2-yl)pyridin-2-yl)butanamide;
2-(6-(cyclopropanesulfonamido)pyridin-2-yl)-N-(5-(6-ethoxypyrazin-2-yl)pyridin-2-yl)butanamide;
N-(4-(5-chloropyridin-3-yl)phenyl)-2-(6-(cyclopropanesulfonamido)pyridin-2-yl)butanamide;
2-(6-(cyclopropanesulfonamido)pyridin-2-yl)-N-(4-(6-ethoxypyrazin-2-yl)-2-fluorophenyl)butanamide;
2-(6-(cyclopropanesulfonamido)pyridin-2-yl)-N-(4-(6-ethoxypyrazin-2-yl)phenyl)butanamide;
2-(6-(cyclopropanesulfonamido)pyrazin-2-yl)-N-(4-(pyridin-3-yl)phenyl)acetamide;
2-(6-(ethylsulfonamido)pyrazin-2-yl)-N-(4-(pyridin-3-yl)phenyl)acetamide;
2-(6-(methylsulfonamido)pyrazin-2-yl)-N-(4-(pyridin-3-yl)phenyl)acetamide;
2-(6-(cyclopropanesulfonamido)pyrazin-2-yl)-N-(5-(6-ethoxypyrazin-2-yl)pyridin-2-yl)-2-methylpropanamide;
2-(6-(cyclopropanesulfonamido)pyrazin-2-yl)-N-(4-(6-ethoxypyrazin-2-yl)phenyl)-2-methylpropanamide;
4-(6-(cyclopropanesulfonamido)pyrazin-2-yl)-N-(5-(6-ethoxypyrazin-2-yl)pyridin-2-yl)tetrahydro-2H-pyran-4-carboxamide;
2-(6-(cyclopropanesulfonamido)pyrazin-2-yl)-N-(5-(6-ethoxypyrazin-2-yl)pyridin-2-yl)-4-methoxy-2-methylbutanamide;
N-(5-(6-ethoxypyrazin-2-yl)pyridin-2-yl)-4-methoxy-2-methyl-2-(6-(methylsulfonamido)pyrazin-2-yl)butanamide;
2-(6-(cyclopropanesulfonamido)pyrazin-2-yl)-N-(5-(6-ethoxypyrazin-2-yl)pyridin-2-yl)-2-fluorobutanamide;
2-(6-(cyclopropanesulfonamido)pyrazin-2-yl)-N-(5-(6-ethoxypyrazin-2-yl)pyridin-2-yl)butanamide;
2-(6-(cyclopropanesulfonamido)pyrazin-2-yl)-N-(4-(6-ethoxypyrazin-2-yl)phenyl)butanamide;
2-(6-(cyclopropanesulfonamido)pyrazin-2-yl)-N-(5-(6-ethoxypyrazin-2-yl)pyridin-2-yl)-2-methoxyacetamide;
2-(6-(cyclopropanesulfonamido)pyrazin-2-yl)-N-(4-(6-ethoxypyrazin-2-yl)phenyl)-2-methoxyacetamide;
2-(6-(cyclopropanesulfonamido)pyrazin-2-yl)-N-(5-(6-ethoxypyrazin-2-yl)pyridin-2-yl)-2-methoxypropanamide;
2-(6-(cyclopropanesulfonamido)pyrazin-2-yl)-N-(5-(6-ethoxypyrazin-2-yl)pyridin-2-yl)-2-(R)-fluorobutanamide;
2-(6-(cyclopropanesulfonamido)pyrazin-2-yl)-N-(5-(6-ethoxypyrazin-2-yl)pyridin-2-yl)-2-(S)-fluorobutanamide;
2-(4-(cyclopropanesulfonamido)pyrimidin-2-yl)-N-(5-(6-ethoxypyrazin-2-yl)pyridin-2-yl)butanamide;
N-(1-(2-(cyclopropanesulfonamido)pyrimidin-4-yl)cyclopropyl)-4-(6-ethoxypyrazin-2-yl)-2-fluorobenzamide;
N-(1-(2-(cyclopropanesulfonamido)pyrimidin-4-yl)propyl)-5-(6-ethoxypyrazin-2-yl)picolinamide;
N-(1-(2-(cyclopropanesulfonamido)pyrimidin-4-yl)propyl)-2-fluoro-4-(5-(trifluoromethyl)pyridin-3-yl)benzamide;
4-(5-chloropyridin-3-yl)-N-(1-(2-(cyclopropanesulfonamido)pyrimidin-4-yl)propyl)-2-fluorobenzamide;
N-(1-(2-(cyclopropanesulfonamido)pyrimidin-4-yl)propyl)-4-(5-(trifluoromethyl)pyridin-3-yl)benzamide;
4-(5-chloropyridin-3-yl)-N-(1-(2-(cyclopropanesulfonamido)pyrimidin-4-yl)propyl)benzamide;
N-(1-(2-(cyclopropanesulfonamido)pyrimidin-4-yl)propyl)-4-(6-ethoxypyrazin-2-yl)-2-(trifluoromethyl)benzamide;
N-(1-(2-(cyclopropanesulfonamido)pyrimidin-4-yl)propyl)-4-(6-ethoxypyrazin-2-yl)-2-fluorobenzamide;
N-(1-(2-(cyclopropanesulfonamido)pyrimidin-4-yl)propyl)-4-(6-(trifluoromethyl)pyrazin-2-yl)benzamide;
N-(1-(2-(cyclopropanesulfonamido)pyrimidin-4-yl)propyl)-4-(6-isopropoxypyrazin-2-yl)benzamide;
N-(1-(2-(cyclopropanesulfonamido)pyrimidin-4-yl)propyl)-4-(6-ethoxypyrazin-2-yl)benzamide;
N-(2-(2-(cyclopropanesulfonamido)pyrimidin-4-yl)butan-2-yl)-4-(6-ethoxypyrazin-2-yl)-2-fluorobenzamide;
N-(2-(6-(cyclopropanesulfonamido)pyrazin-2-yl)propan-2-yl)-2-fluoro-4-(6-isopropoxypyrazin-2-yl)benzamide;
N-(2-(6-(cyclopropanesulfonamido)pyrazin-2-yl)propan-2-yl)-4-(6-(trifluoromethyl)pyrazin-2-yl)benzamide;
N-(1-(6-(cyclopropanesulfonamido)pyrazin-2-yl)propyl)-4-(6-ethoxypyrazin-2-yl)-2-fluorobenzamide;
N-(1-(6-(cyclopropanesulfonamido)pyrazin-2-yl)propyl)-4-(6-ethoxypyrazin-2-yl)-2-(R)-fluorobenzamide; and
N-(1-(6-(cyclopropanesulfonamido)pyrazin-2-yl)propyl)-4-(6-ethoxypyrazin-2-yl)-2-(S)-fluorobenzamide;
   or a pharmaceutically acceptable salt and/or pharmaceutically acceptable solvate thereof.

Such CTPS1 inhibitors are disclosed in PCT publication number WO2019179652 which is incorporated by reference in its entirety for the purpose of the CTPS1 inhibitors disclosed therein. In particular a CTPS1 inhibitor may be a compound described in any one of clauses 1 to 148 of WO2019179652 or a pharmaceutically acceptable salt and/or pharmaceutically acceptable solvate thereof, in particular a compound P1 to P225 or a pharmaceutically acceptable salt and/or pharmaceutically acceptable solvate thereof.

Such CTPS1 inhibitors are also disclosed in PCT publication number WO2019180244 which is incorporated by reference in its entirety for the purpose of the CTPS1 inhibitors disclosed therein. In particular a CTPS1 inhibitor may be a compound described in any one of clauses 1 to 148 of WO2019180244 or a pharmaceutically acceptable salt and/or pharmaceutically acceptable solvate thereof, in particular a compound P1 to P225 or a pharmaceutically acceptable salt and/or pharmaceutically acceptable solvate thereof.

Especially of interest are compounds or a pharmaceutically acceptable salt and/or pharmaceutically acceptable solvate thereof in PCT publication number WO2019180244 which are selective for CTPS1 over CTPS2 (e.g. human CTPS1 over human CTPS2), such as those identified in Table 19. Of particular interest are those compounds or a pharmaceutically acceptable salt and/or pharmaceutically acceptable solvate thereof shown in Table 19 with a selectivity of >60 fold in WO2019180244.

More suitably, the CTPS1 inhibitor is a compound of formula (IV):

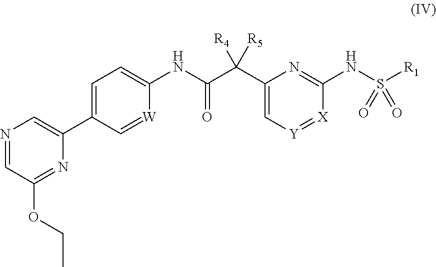

wherein:
(a) when R₄, R₅, X, Y and R₁ are as follows:

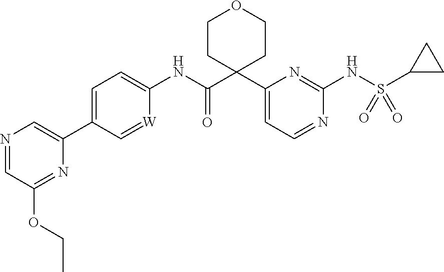

then W is N, CH or CF;
(b) when R₄, R₅, X, W and R₁ are as follows:

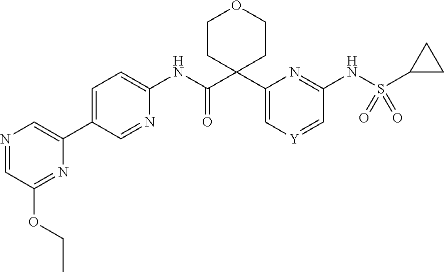

then Y is CH or N;
(c) when W, X, Y and R₁ are as follows:

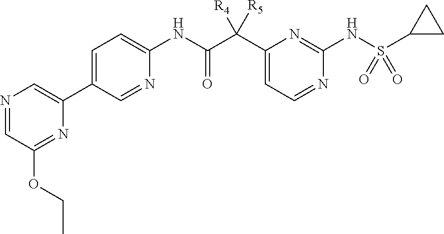

then R₄ and R₅ are joined to form the following structures:

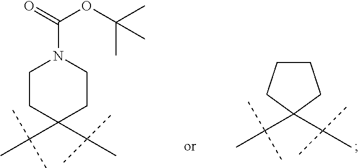

(d) when W, R₄, R₅, X and Y are as follows:

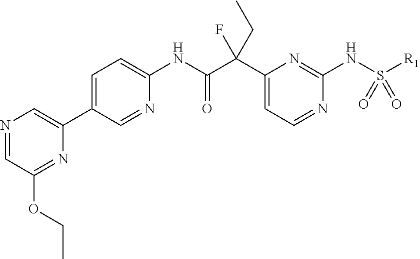

then R₁ is methyl or cyclopropyl; and
(e) the compound is selected from the group consisting of:

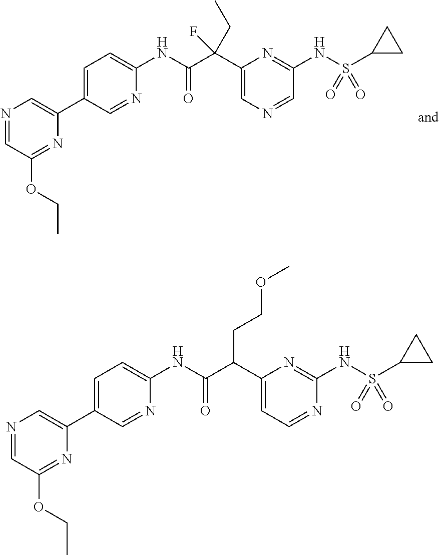 [SMILES: CCOc1cncc(-c2ccc(NC(=O)C(F)(CC)c3cncc(NS(=O)(=O)C4CC4)n3)nc2)n1 ; CCOc1cncc(-c2ccc(NC(=O)C(CCOC)c3ccnc(NS(=O)(=O)C4CC4)n3)nc2)n1]

or a pharmaceutically acceptable salt and/or pharmaceutically acceptable solvate thereof.

More suitably the CTPS1 inhibitor is selected from the following (‘List 0’) compounds:
(R)-2-(2-(cyclopropanesulfonamido)pyrimidin-4-yl)-N-(5-(6-ethoxypyrazin-2-yl)pyridin-2-yl)-2-fluorobutanamide;
(S)-2-(2-(cyclopropanesulfonamido)pyrimidin-4-yl)-N-(5-(6-ethoxypyrazin-2-yl)pyridin-2-yl)-2-fluorobutanamide;
4-(2-(cyclopropanesulfonamido)pyrimidin-4-yl)-N-(5-(6-ethoxypyrazin-2-yl)pyridin-2-yl)tetrahydro-2H-pyran-4-carboxamide;
1-(2-(cyclopropanesulfonamido)pyrimidin-4-yl)-N-(5-(6-ethoxypyrazin-2-yl)pyridin-2-yl)cyclopentane-1-carboxamide;
4-(2-(cyclopropanesulfonamido)pyrimidin-4-yl)-N-(4-(6-ethoxypyrazin-2-yl)phenyl)tetrahydro-2H-pyran-4-carboxamide;
tert-butyl 4-(2-(cyclopropanesulfonamido)pyrimidin-4-yl)-4-((5-(6-ethoxypyrazin-2-yl)pyridin-2-yl)carbamoyl)piperidine-1-carboxylate;
4-(2-(cyclopropanesulfonamido)pyrimidin-4-yl)-N-(4-(6-ethoxypyrazin-2-yl)-2-fluorophenyl)tetrahydro-2H-pyran-4-carboxamide;
2-(2-(cyclopropanesulfonamido)pyrimidin-4-yl)-N-(5-(6-ethoxypyrazin-2-yl)pyridin-2-yl)-4-methoxybutanamide;
(R)-N-(5-(6-ethoxypyrazin-2-yl)pyridin-2-yl)-2-fluoro-2-(2-(methylsulfonamido)pyrimidin-4-yl)butanamide;
(S)-N-(5-(6-ethoxypyrazin-2-yl)pyridin-2-yl)-2-fluoro-2-(2-(methylsulfonamido)pyrimidin-4-yl)butanamide;
4-(6-(cyclopropanesulfonamido)pyridin-2-yl)-N-(5-(6-ethoxypyrazin-2-yl)pyridin-2-yl)tetrahydro-2H-pyran-4-carboxamide;
4-(6-(cyclopropanesulfonamido)pyrazin-2-yl)-N-(5-(6-ethoxypyrazin-2-yl)pyridin-2-yl)tetrahydro-2H-pyran-4-carboxamide;
(R)-2-(6-(cyclopropanesulfonamido)pyrazin-2-yl)-N-(5-(6-ethoxypyrazin-2-yl)pyridin-2-yl)-2-fluorobutanamide; and
(S)-2-(6-(cyclopropanesulfonamido)pyrazin-2-yl)-N-(5-(6-ethoxypyrazin-2-yl)pyridin-2-yl)-2-fluorobutanamide;
   or a pharmaceutically acceptable salt and/or a pharmaceutically acceptable solvate thereof.

Such CTPS1 inhibitors are disclosed in PCT publication number WO2020083975 which is incorporated by reference in its entirety for the purpose of the CTPS1 inhibitors disclosed therein. In particular a CTPS1 inhibitor may be a compound selected from P112, P113, P114, P115, P136, P137, P139, P143, P145, P165, P166, P186, P197, P206 and P207 or a pharmaceutically acceptable salt and/or pharmaceutically acceptable solvate thereof.

Especially of interest are compounds or a pharmaceutically acceptable salt and/or pharmaceutically acceptable solvate thereof in PCT publication number WO2020083975 which are selective for CTPS1 over CTPS2 (e.g. human CTPS1 over human CTPS2), such as those identified in Table 11. Of particular interest are those compounds or a pharmaceutically acceptable salt and/or pharmaceutically acceptable solvate thereof shown in Table 11 with a selectivity of >60 fold in WO2020083975.

Alternatively, the CTPS1 inhibitor is a compound of formula (N:

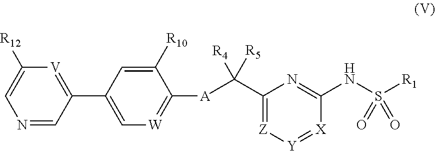

(a) when A, V, W, X, Y, Z, R₁, R₁₀ and R₁₂ are as follows:

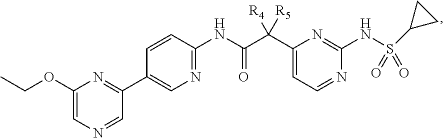

then R₄ and R₅ together with the carbon atom to which they attached form:

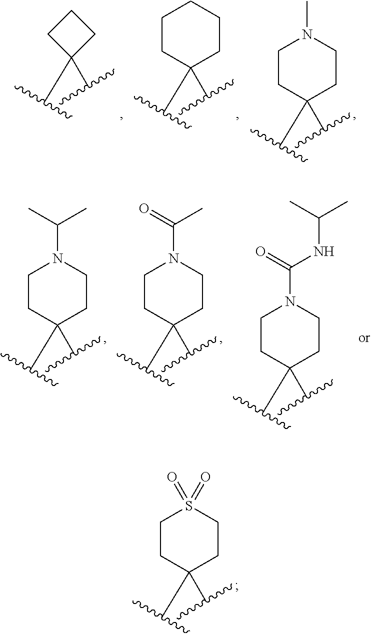

or
(b) when A, V, W, X, Y, Z, R₁, R₁₀ and R₁₂ are as follows:

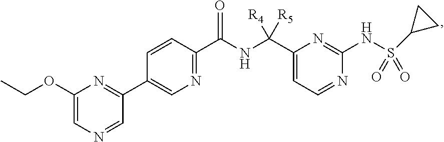

then R₄ and R₅ together with the carbon atom to which they are attached form:

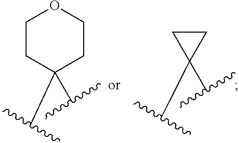 [SMILES: *C1(*)CCOCC1 ; *C1(*)CC1]

or
(c) when A, V, W, X, Y, Z, R₄, R₅, R₁₀ and R₁₂ are as follows:

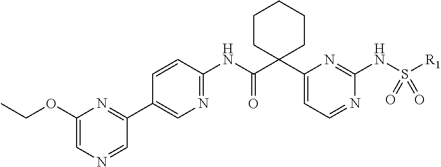

then R₁ is

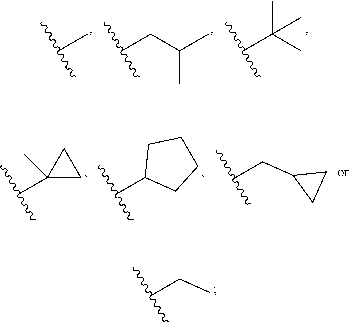

or
(d) when A, V, W, X, Y, Z, R₄, R₅, R₁₀ and R₁₂ are as follows:

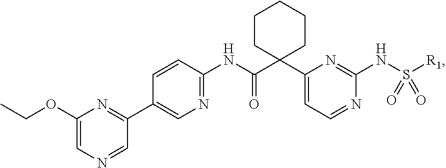

then R₁ is;

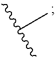

or
(e) when A, X, Y, Z, R₁, R₄ and R₅ are as follows:

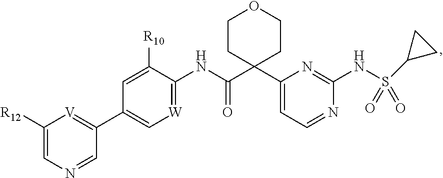

then V, W, R₁₀ and R₁₂ are:

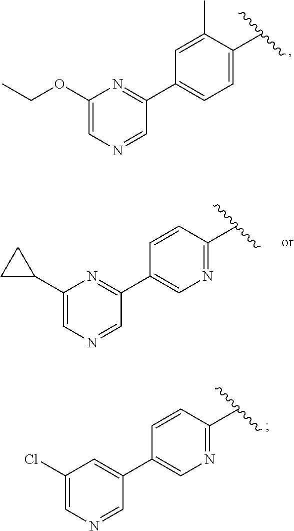 [SMILES: *c1ccc(-c2cncc(OCC)n2)cc1C ; *c1ccc(-c2cncc(C3CC3)n2)cn1 ; *c1ccc(-c2cncc(Cl)c2)cn1]

or
(f) when A, V, W, R₁, R₄, R₅, R₁₀ and R₁₂ are as follows:

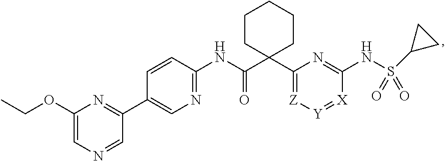

then Z, X and Y are

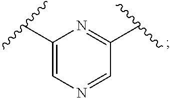 [SMILES: *c1cncc(*)n1]

or
(g) when A, V, W, R₁, R₄, R₅, R₁₀ and R₁₂ are as follows:

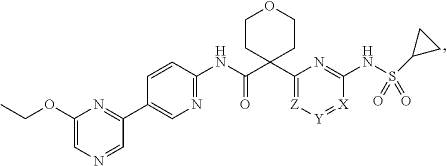

then Z, X and Y are

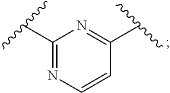 [SMILES: *c1ccnc(*)n1]

or
(h) when A, V, W, R₁, R₄, R₅, R₁₀ and R₁₂ are as follows

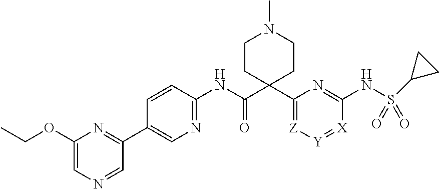

then Z, X and Y are

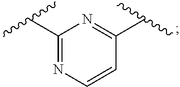 [SMILES: *c1ccnc(*)n1]

or a pharmaceutically acceptable salt and/or pharmaceutically acceptable solvate thereof.

More suitably the CTPS1 inhibitor is selected from the following (‘List E’) compounds:
N-(5-(6-ethoxypyrazin-2-yl)pyridin-2-yl)-4-(2-(methylsulfonamido)pyrimidin-4-yl)tetrahydro-2H-pyran-4-carboxamide;
1-(2-(cyclopropanesulfonamido)pyrimidin-4-yl)-N-(5-(6-ethoxypyrazin-2-yl)pyridin-2-yl)cyclohexane-1-carboxamide;
N-(5-(6-ethoxypyrazin-2-yl)pyridin-2-yl)-1-(2-(methylsulfonamido)pyrimidin-4-yl)cyclohexane-1-carboxamide;
1-(6-(cyclopropanesulfonamido)pyrazin-2-yl)-N-(5-(6-ethoxypyrazin-2-yl)pyridin-2-yl)cyclohexane-1-carboxamide;
4-(2-(cyclopropanesulfonamido)pyrimidin-4-yl)-N-(4-(6-ethoxypyrazin-2-yl)-2-methylphenyl)tetrahydro-2H-pyran-4-carboxamide;
1-(2-(cyclopropanesulfonamido)pyrimidin-4-yl)-N-(5-(6-ethoxypyrazin-2-yl)pyridin-2-yl)cyclobutane-1-carboxamide;
4-(4-(cyclopropanesulfonamido)pyrimidin-2-yl)-N-(5-(6-ethoxypyrazin-2-yl)pyridin-2-yl)tetrahydro-2H-pyran-4-carboxamide;
4-(2-(cyclopentanesulfonamido)pyrimidin-4-yl)-N-(5-(6-ethoxypyrazin-2-yl)pyridin-2-yl)tetrahydro-2H-pyran-4-carboxamide;
N-(5-(6-ethoxypyrazin-2-yl)pyridin-2-yl)-4-(2-((1-methylcyclopropane)-1-sulfonamido)pyrimidin-4-yl)tetrahydro-2H-pyran-4-carboxamide;
4-(2-(Cyclopropanesulfonamido)pyrimidin-4-yl)-N-(5-(6-ethoxypyrazin-2-yl)pyridin-2-yl)-1-methylpiperidine-4-carboxamide;
4-(2-(Cyclopropanesulfonamido)pyrimidin-4-yl)-N-(5-(6-ethoxypyrazin-2-yl)pyridin-2-yl)-1-isopropylpiperidine-4-carboxamide;
4-(2-(Cyclopropanesulfonamido)pyrimidin-4-yl)-N4-(5-(6-ethoxypyrazin-2-yl)pyridin-2-yl)-N1-isopropylpiperidine-1,4-dicarboxamide;
4-(2-((1,1-dimethylethyl)sulfonamido)pyrimidin-4-yl)-N-(5-(6-ethoxypyrazin-2-yl)pyridin-2-yl)tetrahydro-2H-pyran-4-carboxamide;
N-(4-(2-(cyclopropanesulfonamido)pyrimidin-4-yl)tetrahydro-2H-pyran-4-yl)-5-(6-ethoxypyrazin-2-yl)picolinamide;
1-Acetyl-4-(2-(cyclopropanesulfonamido)pyrimidin-4-yl)-N-(5-(6-ethoxypyrazin-2-yl)pyridin-2-yl)piperidine-4-carboxamide;
N-(5-(6-ethoxypyrazin-2-yl)pyridin-2-yl)-4-(2-((2-methylpropyl)sulfonamido)pyrimidin-4-yl)tetrahydro-2H-pyran-4-carboxamide;
4-(2-(cyclopropanesulfonamido)pyrimidin-4-yl)-N-(5-(6-cyclopropylpyrazin-2-yl)pyridin-2-yl)tetrahydro-2H-pyran-4-carboxamide;
N-(5′-chloro-[3,3′-bipyridin]-6-yl)-4-(2-(cyclopropanesulfonamido)pyrimidin-4-yl)tetrahydro-2H-pyran-4-carboxamide;
N-(1-(2-(cyclopropanesulfonamido)pyrimidin-4-yl)cyclopropyl)-5-(6-ethoxypyrazin-2-yl)picolinamide;
4-(2-(cyclopropanesulfonamido)pyrimidin-4-yl)-N-(5-(6-ethoxypyrazin-2-yl)pyridin-2-yl)tetrahydro-2H-thiopyran-4-carboxamide 1,1-dioxide;
N-(5-(6-ethoxypyrazin-2-yl)pyridin-2-yl)-4-(2-(ethylsulfonamido)pyrimidin-4-yl)tetrahydro-2H-pyran-4-carboxamide;
4-(2-(cyclopropylmethylsulfonamido)pyrimidin-4-yl)-N-(5-(6-ethoxypyrazin-2-yl)pyridin-2-yl)tetrahydro-2H-pyran-4-carboxamide and
4-(4-(cyclopropanesulfonamido)pyrimidin-2-yl)-N-(5-(6-ethoxypyrazin-2-yl)pyridin-2-yl)-1-methylpiperidine-4-carboxamide;
   or a pharmaceutically acceptable salt and/or pharmaceutically acceptable solvate thereof.

Such CTPS1 inhibitors are disclosed in PCT publication number WO2020245664 which is incorporated by reference in its entirety for the purpose of the CTPS1 inhibitors disclosed therein. In particular a CTPS1 inhibitor may be a compound selected from P319, P231 to P234, P236, P237, P238, P239, P240, P241, P243, P245, P246, P247, P249, P250, P252, P253, P257, P259, P262, P263 and P140 or a pharmaceutically acceptable salt and/or pharmaceutically acceptable solvate thereof.

Alternatively, the CTPS1 inhibitor is a compound of formula (VI):

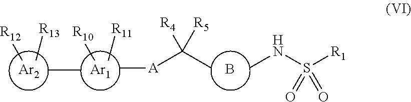

wherein ring B is selected from the group consisting of:

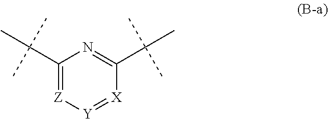

wherein X, Y and Z are as defined below; and

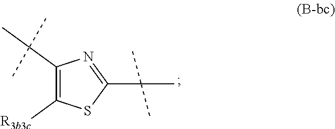

wherein R_{3b3c} is R_{3b} or R_{3c} as defined below;

wherein when B is (B-a) the compound of formula (VI) is a compound of formula (VI-a):

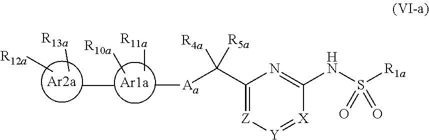

wherein:
Aₐ is Aₐₐ or A_{ba};
   wherein:
   Aₐₐ is an amine linker having the following structure: —NH—, —CH₂NH—or —NHCH₂—;
   A_{ba} is an amide linker having the following structure: —C(═O)NH— or —NHC(═O)—;
X is N or CH;
Y is N or CR₂ₐ;
Z is N or CR₃ₐ,
   with the proviso that when at least one of X or Z is N, Y cannot be N;
R₂ₐ is H, halo, C₁₋₂alkyl, OC₁₋₂alkyl, C₁₋₂haloalkyl or OC₁₋₂haloalkyl; and
R₃ₐ is H, halo, CH₃, OCH₃, CF₃ or OCF₃;
   wherein at least one of R₂ₐ and R₃ₐ is H;
R₁ₐ is R₁ₐₐ or R_{1ba};
   wherein:
   R₁ₐₐ is NR₃₂ₐR₃₃ₐ;
   R_{1ba} is C₁₋₅alkyl, C₀₋₂alkyleneC₃₋₅cycloalkyl which cycloalkyl is optionally substituted by CH₃, or CF₃;
R₄ₐ and R₅ₐ are R₄ₐₐ and R₅ₐₐ, or R_{4ba} and R_{5ba};
   wherein:
   R₄ₐₐ and R₅ₐₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
   R_{4ba} and R_{5ba} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl; and when Aₐ is —NHC(═O)— or —NHCH₂—:
   R_{4ba} and R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN;
R₁₁ₐ is H, F, Cl, C₁₋₂alkyl, CF₃, OCH₃ or CN;
R₁₂ₐ is attached to Ar2 in the ortho or meta position relative to Ar1a and R₁₂ₐ is H, halo,
C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ; and
   when Aₐ is —NHC(═O)—, —NH— or —NHCH₂—; R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O—);
R₁₃ₐ is H or halo;
R₂₁ₐ is H, C₁₋₅alkyl, C(O)C₁₋₅alkyl, C(O)OC₁₋₅alkyl, C₁₋₃alkylOC₁₋₂alkyl, C₁₋₄haloalkyl, or
C₄₋₆heterocycloalkyl;
R₂₂ₐ is H or CH₃;
R₂₃ₐ is H or C₁₋₂alkyl; and
R₂₄ₐ is H or C₁₋₂alkyl
R₂₉ₐ is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl;
R₃₂ₐ is C₁₋₃alkyl and R₃₃ is C₁₋₃alkyl; or
R₃₂ₐ and R₃₃ₐ together with the nitrogen atom to which they are attached form a C₃₋₅heterocycloalkyl;

wherein
R₁ₐ is R₁ₐₐ; and/or
R₄ₐ and R₅ₐ are R₄ₐₐ and R₅ₐₐ; and/or
Aₐ is Aₐₐ; and

wherein when B is (B-bc) and R_{3b3c} is R_{3b}, the compound of formula (VI) is a compound of formula (VI-b):

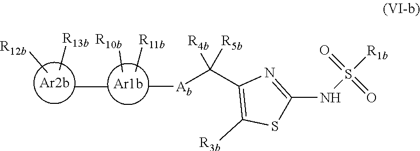

wherein:
A_{b} is A_{ab} or A_{bb};
   wherein:
      A_{ab} is —NR_{6b}CH₂— or —NR_{6b}—;
      A_{bb} is —NR_{6b}C(═O)—;
R_{1b} is R_{1ab} or R_{1bb};
wherein:
   R_{1ab} is NR_{32b}R_{33b};
   R_{1bb} is C₁₋₅alkyl, C₀₋₂alkyleneC₃₋₅cycloalkyl which cycloalkyl is optionally
   substituted by CH₃, C₁₋₃alkyleneOC₁₋₂alkyl, or CF₃;
R_{3b} is H, halo, CH₃, OC₁₋₂alkyl or CF₃;
or R_{3b} together with R_{5bb} forms a 5- or 6-membered cycloalkyl or 5 or 6 membered oxygen-containing heterocycloalkyl;
R_{4b} and R_{5b} are either R_{4ab} and R_{5ab} or R_{4bb} and R_{5bb};
wherein:
   R_{4ab} and R_{5ab} together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆ heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21b}R_{22b}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
   R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆cheterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
   R_{4ab} and R_{5ab} together with the carbon atom to which they are attached form a C₃₋₆heterocycloalkyl comprising one nitrogen atom, wherein said nitrogen atom is substituted by —S(O)₂R_{29b}; or
   R_{4bb} and R_{5bb} are each independently H, halo, C₁₋₆alkyl, C₀₋₂alkyleneC₃₋₆cycloalkyl, C₀₋₂alkyleneC₃₋₆heterocycloalkyl, OC₁₋₆alkyl, OC₀₋₂alkyleneC₃₋₆cycloalkyl, C₁₋₃alkyleneOC₁₋₃alkyl, C₁₋₆alkylOH, C₁₋₆haloalkyl, OC₁₋₆haloalkyl or NR_{21b}R_{22b},
      or R_{4bb} is H and R_{5bb} together with R_{3b} form a 5- or 6-membered cycloalkyl or 5 or 6 membered oxygen-containing heterocycloalkyl,
      or R_{4bb} and R_{5bb} together with the carbon atom to which they are attached form a C₃₋₆cycloalkyl or C₃₋₆heterocycloalkyl,
      or R_{4bb} is H and R_{5bb} and R_{6b} are a C₂₋₃alkylene chain forming a 5- or 6-membered ring;
      or R_{4bb} is 0 and R_{5bb} is absent;
R_{6b} is H or C₁₋₃alkyl,
   or R_{6b} together with R_{11b} when in the ortho-position to group A_{b} are a C₂alkylene chain forming a 5-membered ring,
   or R_{5bb} and R_{6b} are a C₂₋₃alkylene chain forming a 5- or 6-membered ring and R_{4bb} is H;
Ar1 b is 6-membered aryl or heteroaryl;
Ar2b is a 6-membered aryl or heteroaryl and is attached to Ar1b in the para position relative to group A_{b};
R_{10b} is H, halo, C₁₋₃alkyl, OC₁₋₂alkyl, C₁₋₂haloalkyl, OC₁₋₂haloalkyl or CN;
R_{11b} is H, F, Cl, CH₃, ethyl, OCH₃, CF₃, OCF₃ or CN,
   or R_{11b}, when in the ortho-position to group A_{b}, together with R_{6b} are a C₂alkylene chain forming a 5-membered ring;
R_{12b} is attached to Ar2b in the ortho or meta position relative to Ar1 b and R_{12b} is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR_{23b}R_{24b}, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2b, or R_{12b} together with a nitrogen atom to which it is attached forms an N-oxide (N⁺—O—); R_{13b} is H, halo, CH₃ or OCH₃;
R_{21b} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22b} is H or CH₃;
R_{23b} is H or C₁₋₂alkyl;
R_{24b} is H or C₁₋₂alkyl;
R_{29b} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32b} is C₁₋₃alkyl and R_{33b} is C₁₋₃alkyl; or
R_{32b} and R_{33b} together with the nitrogen atom to which they are attached form a C₃₋₅heterocycloalkyl;

wherein:
R_{1b} is R_{1ab}; and/or
R_{4b} and R_{5b} are R_{4ab} and R_{5ab}; and/or
A is A_{ab}; or

wherein when B is (B-bc) and R_{3b3c} is R_{3c}, the compound of formula (VI) is a compound of formula (VI-c):

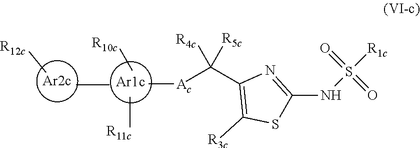

wherein:
A_{c} is A_{ac} or A_{bc};
   wherein:
   A_{ac} is —CH₂NR_{6c}—;
   A_{bc} is —C(═O)NR_{6c}—;
R_{1c} is R_{1ac} or R_{1bc};
   wherein:
   R_{1ac} Is NR_{32c}R_{33c};
R_{1bc} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3c} is H, CH₃, halo, OC₁₋₂alkyl or CF₃;
R_{4c} and R_{5c} are either R_{4ac} and R_{5ac} or R_{4bc} and R_{5bc};
wherein:
   R_{4ac} and R_{5ac} together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c} R_{22c}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
   R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆cheterocycloalkyl ring and a further C₃cycloalkyl ring or a C₃₋₆heterocycloalkyl ring, and wherein
   the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
   R_{4ac} and R_{5ac} together with the carbon atom to which they are attached form a C₃₋₆heterocycloalkyl comprising one nitrogen atom, wherein said nitrogen atom is substituted by —S(O)₂R_{29c}; or
   R_{4bc} and R_{5bc} are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl,
   or R_{4bc} and R_{5bc} together with the carbon atom to which they are attached form a C₃₋₆cycloalkyl or C₃₋₆heterocycloalkyl ring;
R_{6c} is H or C₁₋₃alkyl;
Ar1c is a 6-membered aryl or heteroaryl;
Ar2c is a 6-membered aryl or heteroaryl and is attached to Ar1c in the para position relative to group A_{c};
R_{10c} is H, halo, C₁₋₃alkyl, OC₁₋₂alkyl, C₁₋₂haloalkyl, OC₁₋₂haloalkyl or CN;
R_{11c} is H, F, Cl, CH₃, ethyl, OCH₃, CF₃, OCF₃ or CN;
R_{12c} is attached to Ar2c in the meta or ortho position relative to Ar1c and R_{12c} is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR_{23c} R_{24c}, SO₂CH₃, C(O)N(CH₃)₂, NHC(O)C₁₋₃alkyl, or a C₃₋₆heterocycloalkyl comprising one nitrogen located at the point of attachment to Ar2c, or R_{12c} together with a nitrogen atom to which it is attached forms an N-oxide (N⁺—O⁻);
R_{21c} is H, C₁₋₅alkyl, C(O)C₁₋₅alkyl, C(O)OC₁₋₅alkyl, C₁₋₃alkylOC₁₋₂alkyl, C₁₋₄haloalkyl, or
C₄₋₆heterocycloalkyl;
R_{22c} is H or CH₃;
R_{23c} is H or C₁₋₂alkyl;
R_{24c} is H or C₁₋₂alkyl;
R_{29c} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32c} is C₁₋₃alkyl and R_{33c} is C₁₋₃alkyl; or
R_{32c} and R_{33c} together with the nitrogen atom to which they are attached form a C₃₋₅heterocycloalkyl;
wherein:
R_{1c} is R_{1ac}; and/or
R_{4c} and R_{5c} are R_{4ac} and R_{5ac}; and/or
A_{c} is A_{ac};

or a pharmaceutically acceptable salt and/or pharmaceutically acceptable solvate thereof.

More suitably the CTPS1 inhibitor is selected from the following (‘List F’) compounds:
1-(2-(cyclopropanesulfonamido)pyrimidin-4-yl)-N-(5-(6-ethoxypyrazin-2-yl)pyridin-2-yl)-4-oxocyclohexanecarboxamide;
1-(2-(cyclopropanesulfonamido)pyrimidin-4-yl)-N-(5-(6-ethoxypyrazin-2-yl)pyridin-2-yl)-4-hydroxycyclohexanecarboxamide;
1-(2-(cyclopropanesulfonamido)pyrimidin-4-yl)-N-(5-(6-ethoxypyrazin-2-yl)pyridin-2-yl)-4-hydroxycyclohexanecarboxamide (diastereomer 1);
1-(2-(cyclopropanesulfonamido)pyrimidin-4-yl)-N-(5-(6-ethoxypyrazin-2-yl)pyridin-2-yl)-4-hydroxycyclohexanecarboxamide (diastereomer 2);
1-(2-(cyclopropanesulfonamido)pyrimidin-4-yl)-4-(dimethylamino)-N-(5-(6-ethoxypyrazin-2-yl)pyridin-2-yl)cyclohexane-1-carboxamide;
1-(2-(cyclopropanesulfonamido)pyrimidin-4-yl)-4-(dimethylamino)-N-(5-(6-ethoxypyrazin-2-yl)pyridin-2-yl)cyclohexane-1-carboxamide (diastereomer 1);
1-(2-(cyclopropanesulfonamido)pyrimidin-4-yl)-4-(dimethylamino)-N-(5-(6-ethoxypyrazin-2-yl)pyridin-2-yl)cyclohexane-1-carboxamide (diastereomer 2);
N-(4-(1-((4-(6-Ethoxypyrazin-2-yl)-2-fluorobenzyl)amino)propyl)pyrimidin-2-yl)cyclopropanesulfonamide;
1-(2-(cyclopropanesulfonamido)pyrimidin-4-yl)-N-(5-(6-ethoxypyrazin-2-yl)pyridin-2-yl)-4,4-difluorocyclohexane-1-carboxamide;
8-(2-(cyclopropanesulfonamido)pyrimidin-4-yl)-N-(5-(6-ethoxypyrazin-2-yl)pyridin-2-yl)-1,4-dioxaspiro[4.5]decane-8-carboxamide;
4-(2-((N,N-dimethylsulfamoyl)amino)pyrimidin-4-yl)-N-(5-(6-ethoxypyrazin-2-yl)pyridin-2-yl)tetrahydro-2H-pyran-4-carboxamide;
4-(2-(cyclopropanesulfonamido)pyrimidin-4-yl)-N-(5-(6-ethoxypyrazin-2-yl)pyridin-2-yl)-1-(methylsulfonyl)piperidine-4-carboxamide;
N-(4-(1-(((5-(6-ethoxypyrazin-2-yl)pyridin-2-yl)methyl)amino)cyclopropyl)pyrimidin-2-yl)cyclopropanesulfonamide;
N-(4-(1-((4-(6-ethoxypyrazin-2-yl)-2-fluorobenzyl)amino)cyclopropyl)pyrimidin-2-yl)cyclopropanesulfonamide;
N-(4-(4-(((4-(6-ethoxypyrazin-2-yl)phenyl)amino)methyl)tetrahydro-2H-pyran-4-yl)pyrimidin-2-yl)cyclopropanesulfonamide;
2-(2-(cyclopropanesulfonamido)pyrimidin-4-yl)-N-(5-(6-ethoxypyrazin-2-yl)pyridin-2-yl)-5,8-dioxaspiro[3.4]octane-2-carboxamide;
1-(2-(cyclopropanesulfonamido)pyrimidin-4-yl)-N-(5-(6-ethoxypyrazin-2-yl)pyridin-2-yl)-4-methoxycyclohexane-1-carboxamide;
N-(4-(1-((4-(6-ethoxypyrazin-2-yl)phenyl)amino)propyl)pyrimidin-2-yl)cyclopropanesulfonamidearboxamide;
4-(2-(Cyclopropanesulfonamido)pyrimidin-4-yl)-N-(5-(6-ethoxypyrazin-2-yl)pyridin-2-yl)-1-(2-methoxyacetyl)piperidine-4-carboxamide;
4-(2-(Cyclopropanesulfonamido)pyrimidin-4-yl)-N-(5-(6-ethoxypyrazin-2-yl)pyridin-2-yl)-1-(ethylsulfonyl)piperidine-4-carboxamide;
4-(2-(Cyclopropanesulfonamido)pyrimidin-4-yl)-1-(cyclopropylsulfonyl)-N-(5-(6-ethoxypyrazin-2-yl)pyridin-2-yl)piperidine-4-carboxamide;
4-(2-(Cyclopropanesulfonamido)pyrimidin-4-yl)-1-(N,N-dimethylsulfamoyl)-N-(5-(6-ethoxypyrazin-2-yl) pyridin-2-yl) piperidine-4-carboxamide;
4-(2-(Cyclopropanesulfonamido)pyrimidin-4-yl)-N-(5-(6-ethoxypyrazin-2-yl)pyridin-2-yl)-1-((trifluoromethyl)sulfonyl)piperidine-4-carboxamide;
4-(2-(Cyclopropanesulfonamido)pyrimidin-4-yl)-N-(5-(6-ethoxypyrazin-2-yl)pyridin-2-yl)-1-((1-methyl-1H-pyrazol-3-yl)sulfonyl)piperidine-4-carboxamide;
1-(cyanomethyl)-4-(2-(cyclopropanesulfonamido)pyrimidin-4-yl)-N-(5-(6-ethoxypyrazin-2-yl)pyridin-2-yl)piperidine-4-carboxamide;
ethyl 2-(4-(2-(cyclopropanesulfonamido)pyrimidin-4-yl)-4-((5-(6-ethoxypyrazin-2-yl)pyridin-2-yl)carbamoyl)piperidin-1-yl)acetate;
N-(5-(6-ethoxypyrazin-2-yl) pyridin-2-yl)-4-(2-(ethylsulfonamido)pyrimidin-4-yl)-1-(2-methoxyacetyl)piperidine-4-carboxamide;
N-(5-(6-ethoxypyrazin-2-yl) pyridin-2-yl)-4-(2-(ethylsulfonamido)pyrimidin-4-yl)-1-(methylsulfonyl)piperidine-4-carboxamide;
N-(5-(6-ethoxypyrazin-2-yl)pyridin-2-yl)-4-(2-(ethylsulfonamido)pyrimidin-4-yl)-1-(ethylsulfonyl)piperidine-4-carboxamide;
1-(Cyclopropylsulfonyl)-N-(5-(6-ethoxypyrazin-2-yl)pyridin-2-yl)-4-(2-(ethylsulfonamido)pyrimidin-4-yl)piperidine-4-carboxamide;
N-(5-(6-ethoxypyrazin-2-yl)pyridin-2-yl)-4-(2-(ethylsulfonamido)pyrimidin-4-yl)-1-((1-methyl-1H-pyrazol-3-yl)sulfonyl)piperidine-4-carboxamide;
1-(2-(Cyclopropanesulfonamido)pyrimidin-4-yl)-N-(5-(6-cyclopropylpyrazin-2-yl)pyridin-2-yl)-4-methoxycyclohexane-1-carboxamide (diastereomer 1);
1-(2-(Cyclopropanesulfonamido)pyrimidin-4-yl)-N-(5-(6-cyclopropylpyrazin-2-yl)pyridin-2-yl)-4-methoxycyclohexane-1-carboxamide (diastereomer 2);
1-(2-(Cyclopropanesulfonamido)pyrimidin-4-yl)-N-(5-(6-ethoxypyrazin-2-yl)pyridin-2-yl)-4-(pyrrolidin-1-yl)cyclohexane-1-carboxamide (diastereomer 1);
1-(2-(Cyclopropanesulfonamido)pyrimidin-4-yl)-N-(5-(6-ethoxypyrazin-2-yl)pyridin-2-yl)-4-(pyrrolidin-1-yl)cyclohexane-1-carboxamide (diastereomer 2);
4-amino-1-(2-(cyclopropanesulfonamido)pyrimidin-4-yl)-N-(5-(6-ethoxypyrazin-2-yl)pyridin-2-yl)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-morpholinocyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-morpholinocyclohexane-1-carboxamide (diastereomer 2);
1-(2-(cyclopropanesulfonamido)pyrimidin-4-yl)-N-(5-(6-ethoxypyrazin-2-yl)pyridin-2-yl)-4-(methyl(oxetan-3-yl)amino)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-((2-methoxyethyl)(methyl)amino)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-((2-methoxyethyl)(methyl)amino)cyclohexane-1-carboxamide (diastereomer 2);
1-(2-(cyclopropanesulfonamido)pyrimidin-4-yl)-4-((2,2-difluoroethyl)(methyl)amino)-N-(5-(6-ethoxypyrazin-2-yl)pyridin-2-yl)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-(4-methylpiperazin-1-yl)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-(4-methylpiperazin-1-yl)cyclohexane-1-carboxamide (diastereomer 2);
4-(6-(cyclopropanesulfonamido)pyrazin-2-yl)-N-(5-(6-ethoxypyrazin-2-yl)pyridin-2-yl)-1-(methylsulfonyl)piperidine-4-carboxamide;
4-(4-(cyclopropanesulfonamido)pyrimidin-2-yl)-N-(5-(6-cyclopropylpyrazin-2-yl)pyridin-2-yl)-1-(methylsulfonyl)piperidine-4-carboxamide;
4-(4-(cyclopropanesulfonamido)pyrimidin-2-yl)-N-(5-(6-ethoxypyrazin-2-yl)pyridin-2-yl)-1-(methylsulfonyl)piperidine-4-carboxamide;
4-(4-(cyclopropanesulfonamido)pyrimidin-2-yl)-N-(5-(6-cyclopropylpyrazin-2-yl)pyridin-2-yl)-1-(ethylsulfonyl)piperidine-4-carboxamide; and
4-(4-(cyclopropanesulfonamido)pyrimidin-2-yl)-N-(5-(6-ethoxypyrazin-2-yl)pyridin-2-yl)-1-(ethylsulfonyl)piperidine-4-carboxamide;
   or a pharmaceutically acceptable salt and/or pharmaceutically acceptable solvate thereof.

Such CTPS1 inhibitors are disclosed in PCT publication number WO2020245665 which is incorporated by reference in its entirety for the purpose of the CTPS1 inhibitors disclosed therein. In particular a CTPS1 inhibitor may be a compound described in any one of clauses 1 to 204 of WO2020245665 or a pharmaceutically acceptable salt and/or pharmaceutically acceptable solvate thereof, in particular a compound selected from P226, P227, P228, P229, P230, P235, P242, P244, P248, P251, P254, P255, P256, P258, P260, P261, P288, P289, P290, P291, P292, P293, P294, P295, P296, P297, P298, P299, P300, P301, P302, P303, P304, P305, P306, P307, P308, P309, P310, P311, P312, P313, P314, P315, P316, P317 and P318 or a pharmaceutically acceptable salt and/or pharmaceutically acceptable solvate thereof.

Alternatively, the CTPS1 inhibitor is a compound of formula (VII):

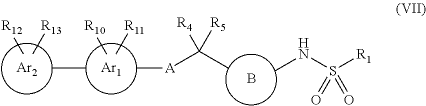

wherein
A is A, or A_{b};
wherein
   Aₐ is an amine linker having the following structure: —NH—, —CH₂NH—or —NHCH₂—;
   A_{b} is an amide linker having the following structure: —C(═O)NH— or —NHC(═O)—;
B is

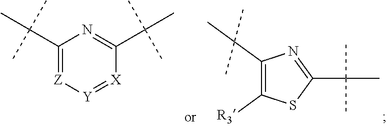

X is N or CH;
   Y is N or CR₂;
   Z is N or CR₃,
      with the proviso that when at least one of X or Z is N, Y cannot be N;
R₁ is C₁₋₅fluoroalkyl, with the proviso that R₁ is not CF₃;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
   wherein at least one of R₂ and R₃ is H;
R₃, is H, halo, CH₃, OC₁₋₂alkyl or CF₃; and
   when A is —NHC(═O)—, additionally R₃, together with R₅ forms a 5- or 6-membered cycloalkyl or 5 or 6 membered oxygen-containing heterocycloalkyl;
R₄ and R₅ are R₄ₐ and R₅ₐ, or R_{4b} and R_{5b};
   wherein
   R₄ₐ and R₅ₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁R₂₂; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl comprising one nitrogen atom, wherein said nitrogen atom is substituted by —S(O)₂R₂₉; or
R_{4b} and R_{5b} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4b} and R_{5b} together with the carbon atom to which they are attached form a C₃₋₆cycloalkyl or C₃₋₆heterocycloalkyl; and
   when A is —NHC(═O)— or —NHCH₂—:
   R_{4b} and R_{5b} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to group A;
R₁₀ is H, halo, C₁₋₃alkyl, C₁₋₂haloalkyl, OC₁₋₂alkyl, OC₁₋₂haloalkyl or CN;
R₁₁ is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
   when A is —NHC(═O)—, —NH— or —NHCH₂—:
   R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O—);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
R₂₉ is C₁₋₃alkyl, C₀₋₂alkyleneC₃₋₅cycloalkyl which cycloalkyl is optionally substituted by CH₃, or CF₃;
R₃₂ is C₁₋₃alkyl and R₃₃ is C₁₋₃alkyl; or
R₃₂ and R₃₃ together with the nitrogen atom to which they are attached form a C₃₋₅heterocycloalkyl;

or a pharmaceutically acceptable salt and/or pharmaceutically acceptable solvate thereof.

More suitably the CTPS1 inhibitor is selected from the following (‘List G’) compounds; 4-(2-((2,2-difluoroethyl)sulfonamido)pyrimidin-4-yl)-N-(5-(6-ethoxypyrazin-2-yl)pyridin-2-yl)tetrahydro-2H-pyran-4-carboxamide; and 2-(2-((2,2-difluoroethyl)sulfonamido)pyrimidin-4-yl)-N-(5-(6-ethoxypyrazin-2-yl)pyridin-2-yl)-2-fluorobutanamide;

or a pharmaceutically acceptable salt and/or pharmaceutically acceptable solvate thereof.

Such CTPS1 inhibitors are disclosed in PCT publication number WO2021053403 which is incorporated by reference in its entirety for the purpose of the CTPS1 inhibitors disclosed therein. In particular a CTPS1 inhibitor may be a compound described in any one of clauses 1 to 191 of WO2021053403 or a pharmaceutically acceptable salt and/or pharmaceutically acceptable solvate thereof, in particular a compound selected from P271 and P284 or a pharmaceutically acceptable salt and/or pharmaceutically acceptable solvate thereof.

Alternatively, the CTPS1 inhibitor is compound of formula (VIII):

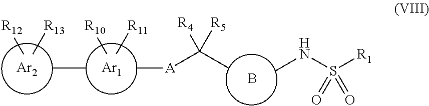

wherein
A is A, or A_{b};
wherein
   Aₐ is an amine linker having the following structure: —NH—, —CH₂NH—or —NHCH₂—;
   A_{b} is an amide linker having the following structure: —C(═O)NH— or —NHC(═O)—;
B is

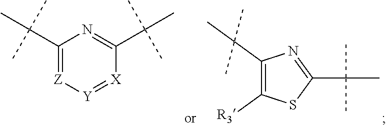

X is N or CH;
   Y is N or CR₂;
   Z is N or CR₃,
      with the proviso that when at least one of X or Z is N, Y cannot be N;
R₁ is C₁₋₅alkyl or C₀₋₂alkyleneC₃₋₅cycloalkyl, which alkyl or (alkylene)cycloalkyl is substituted by CN;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
   wherein at least one of R₂ and R₃ is H;
R₃, is H, halo, CH₃, OC₁₋₂alkyl or CF₃; and
   when A is —NHC(═O)—, additionally R₃ together with R₅ forms a 5- or 6-membered cycloalkyl or 5 or 6 membered oxygen-containing heterocycloalkyl;
R₄ and R₅ are R₄ₐ and R₅ₐ, or R_{4b} and R_{5b};
   wherein
   R₄ₐ and R₅ₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁R₂₂; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
   R₄ₐ and R₅ₐ together with the carbon atom to which they are attached form a C₃₋₆heterocycloalkyl comprising one nitrogen atom, wherein said nitrogen atom is substituted by —S(O)₂R₂₉; or
R_{4b} and R_{5b} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4b} and R_{5b} together with the carbon atom to which they are attached form a C₃₋₆cycloalkyl or C₃₋₆heterocycloalkyl; and
   when A is —NHC(═O)— or —NHCH₂—:
   R_{4b} and R_{5b} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to group A;
R₁₀ is H, halo, C₁₋₃alkyl, C₁₋₂haloalkyl, OC₁₋₂alkyl, OC₁₋₂haloalkyl or CN;
R₁₁ is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
   when A is —NHC(═O)—, —NH— or —NHCH₂—:
   R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O—);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
R₂₉ is C₁₋₃alkyl, C₀₋₂alkyleneC₃₋₅cycloalkyl which cycloalkyl is optionally substituted by CH₃, or CF₃;
R₃₂ is C₁₋₃alkyl and R₃₃ is C₁₋₃alkyl; or
R₃₂ and R₃₃ together with the nitrogen atom to which they are attached form a C₃₋₅heterocycloalkyl;
   or a pharmaceutically acceptable salt and/or pharmaceutically acceptable solvate thereof.

More suitably the CTPS1 inhibitor is selected from the following (‘List H’) compounds:
4-(2-((1-cyanocyclopropane)-1-sulfonamido)pyrimidin-4-yl)-N-(5-(6-ethoxypyrazin-2-yl)pyridin-2-yl)tetrahydro-2H-pyran-4-carboxamide; and
4-(2-((cyanomethyl)sulfonamido)pyrimidin-4-yl)-N-(5-(6-ethoxypyrazin-2-yl)pyridin-2-yl)tetrahydro-2H-pyran-4-carboxamide;
   or a pharmaceutically acceptable salt and/or pharmaceutically acceptable solvate thereof.

Such CTPS1 inhibitors are disclosed in PCT publication number WO2021053402 which is incorporated by reference in its entirety for the purpose of the CTPS1 inhibitors disclosed therein. In particular a CTPS1 inhibitor may be a compound described in any one of clauses 1 to 191 of WO2021053402 or a pharmaceutically acceptable salt and/or pharmaceutically acceptable solvate thereof, in particular a compound selected from P285 and P287 or a pharmaceutically acceptable salt and/or pharmaceutically acceptable solvate thereof.

The CTPS1 inhibitor may be 4-(2-(cyclopropanesulfonamido)pyrimidin-4-yl)-N-(5-(6-ethoxypyrazin-2-yl)pyridin-2-yl)tetrahydro-2H-pyran-4-carboxamide (referred to herein as ‘CTPS-IA’):

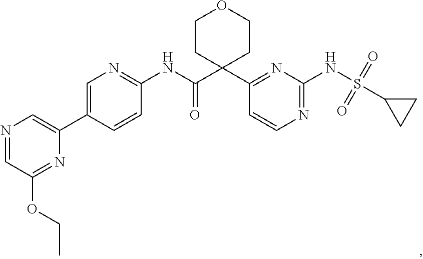 [SMILES: CCOc1cncc(-c2ccc(NC(=O)C3(c4ccnc(NS(=O)(=O)C5CC5)n4)CCOCC3)nc2)n1]

or a pharmaceutically acceptable salt and/or pharmaceutically acceptable solvate thereof. CTPS-IA is a potent and selective inhibitor of CTPS1 (see e.g. WO2020083975).

Alternatively, the CTPS1 inhibitor may be N-(5-(6-ethoxypyrazin-2-yl)pyridin-2-yl)-4-(2-(ethylsulfonamido)pyrimidin-4-yl)tetrahydro-2H-pyran-4-carboxamide (referred to herein as ‘CTPS-IB’):

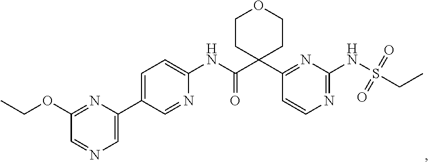 [SMILES: CCOc1cncc(-c2ccc(NC(=O)C3(c4ccnc(NS(=O)(=O)CC)n4)CCOCC3)nc2)n1]

or a pharmaceutically acceptable salt and/or pharmaceutically acceptable solvate thereof. CTPS-IB is a potent and selective inhibitor of CTPS1 (see e.g. WO2020245664).

The compounds described above (and methods for making these compounds) are disclosed in PCT publication numbers WO2019106156, WO2019180244, WO2019106146, WO2019179652, WO2020245665, WO2020245664, WO2021053403, WO2021053402 or WO2020083975.

Alternatively, the CTPS1 inhibitor is a compound of formula (IX):

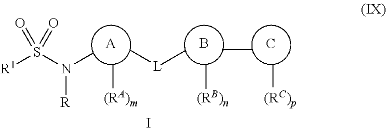

or a pharmaceutically acceptable salt thereof, wherein:
R¹ is selected from C₁₋₆ aliphatic, a 1-7 membered saturated or partially unsaturated monocylic carbocyclic ring; and a 3-7 membered saturated or partially unsaturated monocyclic heterocyclic ring having 1-2 heteroatoms independently selected from nitrogen, oxygen, and sulfur, each of which is substituted with q instances of R^{A};
Ring A is selected from phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur, a 3-7 membered saturated or partially unsaturated monocyclic heterocyclic ring having 1-2 heteroatoms independently selected from nitrogen, oxygen, and sulfur, and a 7-11 membered fused bicyclic heteroaryl ring having 1-4 heteroatoms independently selected from nitrogen, oxygen, and sulfur,

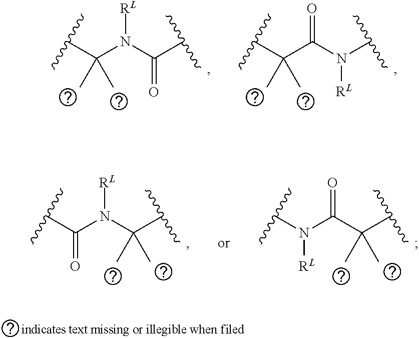

wherein each of R^{L}, R^{L′}, and R^{L″} is independently hydrogen, —CN, halogen, or an optionally substituted group selected from C₁₋₆ aliphatic; phenyl; a 3-7 membered saturated or partially unsaturated monocyclic carbocyclic ring, a 3-7 membered saturated or partially unsaturated monocyclic heterocyclic ring having 1-2 heteroatoms independently selected from nitrogen, oxygen, and sulfur; a 5-6 membered monocyclic heteroaryl ring having 14 heteroatoms independently selected from nitrogen, oxygen, and sulfur, or
two of R^{L} R^{L′}, and R^{L″} groups are taken together with the atoms to which each is attached, to form an optionally substituted 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; or
any one of R^{L} R^{L′}, and R^{L″}, together with R^{B} forms a 7-10 membered saturated or partially unsaturated fused bicyclic ring.
Ring B is selected from phenyl, a 3-7 membered saturated or partially unsaturated monocyclic carbocyclic ring, a 3-6 membered monocyclic heteroaryl ring having 1.4 heteroatoms independently selected from nitrogen, oxygen, and sulfur, a 3-7 membered saturated or partially unsaturated monocyclic heterocyclic ring having 1-3 heteroatoms independently selected from nitrogen, oxygen, and sulfur; a 5-1l membered saturated or partially unsaturated fused, bridged, or spiro, bicyclic carbocyclic ring, a 7-11 membered fused bicyclic aryl ring, a 7-11 membered saturated or partially unsaturated fused, bridged, or spiro, bicyclic heterocyclic, ring having 1-4 heteroatoms independently selected from nitrogen, oxygen, and sulfur; and a 7-11 membered fused bicyclic heteroaryl ring having 1-4 heteroatoms independently selected from nitrogen, oxygen, and sulfur
Ring C is selected from a phenyl, 3-7 membered saturated or partially unsaturated monocyclic carbocylic ring; a 5-6 membered monocyclic heteroaryl ring having 1-4 heteroatoms independently selected from nitrogen, oxygen, and sulfur, a 3-7 membered saturated or partially unsaturated monocyclic heterocyclic ring having 1-2 heteroatoms independently selected from nitrogen, oxygen, and sulfur; and a 7-11 membered fused bicyclic heteroaryl ring having 1-4 heteroatoms independently selected from nitrogen, oxygen, and sulfur; or
the bond between Ring B and Ring C is absent, and Ring B and Ring C together form a 7-11 membered saturated or partially unsaturated fused, bridged, or spiro, bicyclic carbocyclic ring; a 7-11 membered fused bicyclic aryl ring, a 7-11 membered saturated or partially unsaturated fused, bridged, or spiro, bicyclic heterocyclic ring having 1-4 heteroatoms independently selected from nitrogen, oxygen, and sulfur; (r a 7-11 membered fused bicyclic heteroaryl sing having 1-4 heteroatoms independently selected from nitrogen, oxygen, and sulfur;
each instance R^{A}, R^{B}, and R^{C} is independently oxo, halogen. —CN, —NO, —OR, SR, —NR₂, —S(O)₂R, —S(O))NR₂, —S(O)R, —S(O)NR₂, —C(O)R, —C(O)OR, C(O)NR₂, —C(O)B(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)SO)₂NR₂, —N(R)S(O)₂R, —N—S(O)R₂, —S(NR)(O)R, —N(R)S(O)R, —N(R)CN, —P(O)(R)NR₂, —P(O)(R)OR, or —P(O)R₂; or each instance of R^{C} is independently an optionally substituted group selected from C₁₋₆ aliphatic, phenyl; naphthalenyl, a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur, an 8-10 membered bicyclic heteroaryl ring having 14 heteroatoms metaatoms independency selected from nitrogen, oxygen, and sulfur; a 5-8 membered saturated or partially unsaturated bridged bicyclic ring having 0-3 heteroatoms independently selected from nitrogen, oxygen- and sulfur a 6-10 membered saturated or partially unsaturated spirocyclic ring having 0-3 heteroatoms independently selected from nitrogen, oxygen, and sulfur, or a 6-11 membered saturated or partially unsaturated bicyclic heterocyclic ring having 1-2 heteroatoms independently selected from nitrogen, oxygen, and sulfur; each of which is substituted with r instances of R and s instances of R^{D}; or two R^{C} groups are optionally taken together with the atoms to which each R^{C} is attached, to from an optionally substituted 3-7 membered saturated or partially unsaturated monocyclic carbocyclic ring; a 5-7 membered heteroaryl ring having 1-2 heteroatoms independently selected from nitrogen, oxygen, and sulfur; or a 3-7 membered saturated or partially unsaturated monocyclic heterocyclic ring having 1-2 heteroatoms independently selected from nitrogen, oxygen- and sulfur;
each instance of R^{D} is independently oxo, halogen, —CN, —NO₂, —OR, —SR, —NR₂, —S(O)₂R, —S(O)₂NR₂, —S(O)R, —S(O)NR, —C(O)R, —C(O)OR, C(O)NR₂, —C(C))N(R)CR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —NR(R)S(O)₂R, —N═S(O)R₂, —S(NR)(O)R, —N(R)S(O)R, —N(R)CN, —P(O)(R)NR₂, —P(O)(R)OR or —P(O)R₂;
each R is independently hydrogen, —CN, halogen, or an optionally substituted group selected from C₁₋₆ aliphatic; phenyl, naphthalenyl; a 3-7 membered saturated or partially unsaturated monocyclic carbocyclic ring; a 3-7 membered saturated or partially unsaturated monocyclic
heterocyclic ring having 1-2 heteroatoms independently selected from nitrogen, oxygen, and sulfur, a −6 membered monocyclic heteroaryl ring having 1-4 heteroatoms independently selected from nitrogen, oxygen, and sulfur; an 8-14) membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur, a 6-10 membered saturated or partially unsaturated spirocyclic ring having 0-3 heteroatom, independently selected from nitrogen, oxygen, and sulfur, a 6-11 membered saturated m partially unsaturated bicyclic carbocyclic ring having 1-2 heteroatoms independently selected from nitrogen, oxygen, and sulfur, or
two R groups are taken together with the atoms to which each R is attached, to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur, or
m is 0, 1, or 2
n is 0, 1; or 2.
p is 0, 1; or 2,
each q is independently 01, 2, 3, or 4.
each r is independently 0, 1, 2, 3, or 4; and
each s is independently 0, 1, 2, 3, or 4;
provided that when:
   R¹ is C₁₋₆ aliphatic or a 3-7 membered saturated or partially unsaturated monocylic carbocyclic ring;
   the R group of the sulfonamide moiety

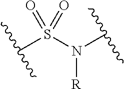

is hydrogen or para-methoxybenzyl;
L is

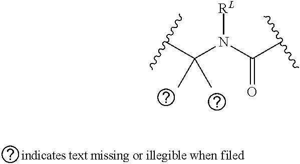

and the R^{L} R^{L′}, and R^{L″} and R^{L′} groups are not taken together with the atoms to which each is attached to form an optionally substituted 3-7 membered saturated or partially unsaturated monocyclic heterocyclic ring having 1-2 heteroatoms independently selected from nitrogen, oxygen, and sulfur, or L is

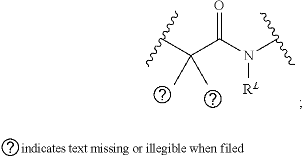

Ring B is phenyl or a 6-membered monocyclic heteraryl ring having 1-4 heteroatoms independently selected from nitrogen, oxygen, and sulfur; and
Ring C is phenyl or a 6-membered monocyclic heteroaryl ring having 1-4 heteroatoms independently selected from nitrogen, oxygen, and sulfur and is attached to Ring B in the parm position relative to the group;
then Ring A and its R^{A} substituents are other than

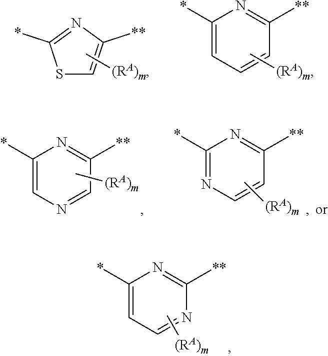

where * denote attachment to the

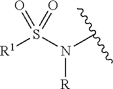

moiety and ** denotes attachment to the

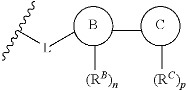

moiety or a pharmaceutically acceptable salt and/or pharmaceutically acceptable solvate thereof.

Such CTPS1 inhibitors are disclosed in PCT publication number WO2022087634 which is incorporated by reference in its entirety for the purpose of the CTPS1 inhibitors disclosed therein. In particular a CTPS1 inhibitor may be a compound described in any one of claims 1 to 31 of WO2022087634 or a pharmaceutically acceptable salt and/or pharmaceutically acceptable solvate thereof. A CTPS1 inhibitor may be a compound selected from compounds I-1 to 1-286 of WO2022087634, or a pharmaceutically acceptable salt and/or pharmaceutically acceptable solvate thereof. A CTPS1 inhibitor may be a compound selected from compounds Z-1 to Z-10 of WO2022087634, or a pharmaceutically acceptable salt and/or pharmaceutically acceptable solvate thereof.

Depending on the nature of the specific CTPS1 inhibitor, the CTPS1 inhibitor may be provided in the form of a pharmaceutically acceptable salt and/or pharmaceutically acceptable solvate. In some embodiments the CTPS1 inhibitor is provided in the form of a pharmaceutically acceptable salt and pharmaceutically acceptable solvate (i.e. a pharmaceutically acceptable solvate of a pharmaceutically acceptable salt). In other embodiments the CTPS1 inhibitor is provided in the form of a pharmaceutically acceptable salt. In further embodiments the CTPS1 inhibitor is provided in the form of a pharmaceutically acceptable solvate. In some embodiments the CTPS1 inhibitor is provided in free form (i.e. not a salt or solvate).

Suitable pharmaceutically acceptable salts will be apparent to those skilled in the art. Pharmaceutically acceptable salts include those Remington's Pharmaceutical Sciences, 17th ed., Mack Publishing Company, Easton, PA, 1985, p.1418. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Pharmaceutically acceptable salts may also be formed with metal ions such as metal salts, such as sodium or potassium salts, and organic bases such as basic amines e.g. with ammonia, meglumine, tromethamine, piperazine, arginine, choline, diethylamine, benzathine or lysine.

The CTPS1 inhibitor may form acid or base addition salts with one or more equivalents of the acid or base. The present invention includes within its scope all possible stoichiometric and non-stoichiometric forms.

The CTPS1 inhibitor may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water).

Unless defined for as part of a formula or compound structure, the CTPS1 inhibitor encompasses all isomers of the CTPS1 inhibitors disclosed herein including all geometric, tautomeric and optical forms, and mixtures thereof (e.g. racemic mixtures). Where additional chiral centres are present, the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The CTPS1 inhibitor encompasses all isotopic forms of the CTPS1 inhibitors provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exist as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

Unnatural variant isotopic forms comprising radioisotopes may, for example, be used for drug and/or substrate tissue distribution studies.

In one embodiment, the CTPS1 inhibitor is provided in a natural isotopic form.

In one embodiment, the CTPS1 inhibitor is provided in an unnatural variant isotopic form. In one embodiment, the CTPS1 inhibitor is provided whereby a single atom of the compound exists in an unnatural variant isotopic form. In another embodiment, the CTPS1 inhibitor is provided whereby two or more atoms exist in an unnatural variant isotopic form.

### Therapeutic Uses and Applications

The invention may be useful in the treatment of cancers which are deficient in CTPS2. Deficient in CTPS2, as used herein, refers to a substantial deficiency in CTPS2 function, in particular a complete loss of CTPS2 function. Deficiency in CTPS2 function may result from a genomic alteration and/or an epigenic change.

Deficiency in CTPS2 function may result from a genomic alteration or alterations e.g.:
(i) complete loss of the CTPS2 gene by genomic deletion;
(ii) partial loss of the CTPS2 gene by genomic deletion;
(iii) disruption of the CTPS2 gene by a structural DNA variant such as an inversion, duplication or translocation falling within the footprint of the gene; or
(iv) mutation of the CTPS2 gene such that CTPS2 expression is substantially reduced, or most suitably completely lost.

Deficiency in CTPS2 function may result from an epigenic change or changes such as altered expression of the CTPS2 gene, for example due to changes in regulatory elements due to altered methylation and/or histone modification, such that CTPS2 expression is substantially reduced, or most suitably completely lost.

Suitably, deficiency in CTPS2 function may result from complete loss of the CTPS2 gene by genomic deletion (homozygous deletion in tumours arising in females or hemizygous deletion in tumours arising in males).

The existence of CTPS2 deficiency will typically be determined by analysis of a sample from a subject. A sample, as used herein, will be understood to be an appropriate sample for the intended analysis methodology i.e. a sample which allows the determination of CTPS2 deficiency or otherwise. For example, the sample may be a biopsy containing cancer cells (e.g. a tumour biopsy) or may be a sample containing circulating cancer cells or may be a sample containing cell free cancer DNA. Samples may be obtained or provided by any suitable method known in the art.

### Methods for Determining CTPS2 Deficiency Include:

(i) methods which detect DNA changes, including analysis of tumour derived DNA, obtained from tumour tissue, circulating tumour cells, cell free DNA or cell free exosomes, using a suitable technique for the detection of genomic alterations likely to result in a substantial deficiency in CTPS2 function, in particular a complete loss of CTPS2 function, including whole genome sequencing, whole exome sequencing, targeted gene sequencing using capture based enrichment, targeted gene sequencing using PCR-based enrichment, real-time quantitative PCR, digital droplet PCR, in situ hybridization or fluorescence in situ hybridization;
(ii) methods which detect RNA changes, including analysis of a suitable sample of RNA, such as acquired from tumour tissue, circulating tumour cells or cell free exosomes, using a suitable technique for the detection of CTPS2 RNA such as RNA sequencing, gene expression array, real-time quantitative PCR, digital droplet PCR or in situ hybridization;
(iii) methods which detect epigenetic changes, including (a) analysis of tumour derived DNA, obtained from tumour tissue, circulating tumour cells or cell free DNA, using a suitable technique for the detection of epigenic changes likely to result in loss of CTPS2 expression, including DNA methylation analysis or characterisation of histone modification or (b) analysis of a suitable sample of RNA, such as acquired from tumour tissue, circulating tumour cells or cell free exosomes, and analysed by RNA sequencing, gene expression array, real-time quantitative PCR, digital droplet PCR or in situ hybridization; and/or
(iv) methods which detect protein changes, including analysis of tumour tissue or circulating tumour cells using a suitable technique for the detection of CTPS2 protein such as immunohistochemistry, flow cytometry or mass cytometry.

It will be appreciated that a plurality of analysis techniques may be applied in combination.

Suitably, the step of identifying that a subject has a cancer which is deficient in CTPS2 comprises (i) applying a suitable method for determining CTPS2 deficiency, such as a technique to detect relevant alteration of DNA, RNA or protein, (ii) analysis of data produced by the method, and (iii) interpretation of the data produced by the method to determine the likelihood of a substantial deficiency in CTPS2 function, in particular a complete loss of CTPS2 function.

For a cancer harbouring a genomic alteration likely to result in a substantial deficiency in CTPS2 function, in particular a complete loss of CTPS2 function, detection may use a suitable sample of DNA acquired from a tissue biopsy, circulating tumour cells or cell free DNA. Suitable techniques for the detection of a genomic alteration likely to result in a substantial deficiency in CTPS2 function include whole genome sequencing, whole exome sequencing, targeted gene sequencing using capture-based enrichment, targeted gene sequencing using PCR-based enrichment, real-time quantitative PCR, digital droplet PCR, in situ hybridization or fluorescence in situ hybridization. In addition, a genomic alteration likely to result in a substantial deficiency in CTPS2 function could be detected from a suitable sample of RNA acquired from a tissue biopsy, circulating tumour cells or cell free exosomes, and analysed by RNA sequencing, gene expression array, real-time quantitative PCR, digital droplet PCR or in situ hybridization. In addition, a genomic alteration likely to result in a substantial deficiency in CTPS2 function could be detected from a suitable sample of tumour cells acquired from a tissue biopsy or circulating tumour cells, and analysed by immunohistochemistry, flow cytometry or mass cytometry.

For a cancer harbouring epigenic changes likely to result in a substantial deficiency of CTPS2, detection will require a suitable sample of DNA acquired from a tissue biopsy, circulating tumour cells or cell free DNA. Suitable techniques for the detection of epigenic changes likely to result in a substantial deficiency of CTPS2 include DNA methylation analysis or characterisation of histone modification. In addition, epigenic changes likely to result in a substantial deficiency of CTPS2 function could be detected from a suitable sample of RNA acquired from a tissue biopsy, circulating tumour cells or cell free exosomes, and analysed by RNA sequencing, gene expression array, real-time quantitative PCR, digital droplet PCR or in situ hybridization. In addition, epigenic changes likely to result in a substantial deficiency of CTPS2 function could be detected from a suitable sample of tumour cells acquired from a tissue biopsy or circulating tumour cells, and analysed by immunohistochemistry, flow cytometry or mass cytometry.

### Susceptibility to CTPS1 Treatment

The present invention may be used to determine if cancer cells or cancers may be susceptible to treatment with a CTPS1 inhibitor. A ‘susceptible’ cancer or cancer cells in this context are those which have been identified as an increased probability of benefiting from treatment according to the invention (e.g. demonstrating high in vivo efficacy, reduction in the dose required for effect in vivo and/or an improved safety profile/reduced side effects) relative to a cancer or cancer cells which are not known to be susceptible to treatment with a CTPS1 inhibitor.

The susceptible cancer or cancer cells may be expected to be deficient in CTPS2 (i.e. more likely than not be deficient in CTPS2), suitably the susceptible cancer or cancer cells are deficient in CTPS2.

Those cancers or cancer cells which have been identified as having an increased probability of being deficient in CTPS2 or which have been identified as expected to be deficient in CTPS2 may, for example, be (i) identified as cancers or cancer cells of a type where deficiency in CTPS2 is more prevalent than among cancers more generally, or (ii) identified as being deficient in CTPS2, or (iii) identified as more likely being deficient in CTPS2 than not, using methodology which may not be definitive.

A susceptible cancer or cancer cells may be identified by analysis of DNA and detection of a genomic alteration of CTPS2 likely to result in substantial loss of CTPS2 activity.

A cancer or cancer cells may be expected to have a genomic alteration of CTPS2 likely to result in substantial loss of CTPS2 activity, rendering them susceptible to treatment with a CTPS1 inhibitor.

A susceptible cancer or cancer cells may be identified by analysis of RNA and detection of reduced expression of CTPS2 likely to result in substantial loss of CTPS2 activity.

A susceptible cancer or cancer cells may be identified by analysis of protein and detection of reduced expression of CTPS2 likely to result in substantial loss of CTPS2 activity.

A cancer or cancer cells may be expected to have reduced expression of CTPS2 likely to result in substantial loss of CTPS2 activity, rendering them susceptible to treatment with a CTPS1 inhibitor.

A susceptible cancer or cancer cells may be identified by analysis of epigenic patterns and detection of changes in DNA methylation or histone modification likely to result in substantial loss of CTPS2 activity.

A cancer or cancer cells may be expected to have changes in DNA methylation or histone modification likely to result in substantial loss of CTPS2 activity, rendering them susceptible to treatment with a CTPS1 inhibitor.

A susceptible cancer or cancer cells may be identified by culture of cancer cells (e.g. human cancer cells) in the presence of a selective CTPS1 inhibitor where the growth patterns of the cancer cells shows sensitivity to CTPS1 inhibition. Sensitivity to CTPS1 inhibition may be due to substantial loss of CTPS2 activity. Selectivity is suitably at least 2-fold, such as at least 30-fold, especially at least 60-fold and in particular at least 1000-fold, especially for human CTPS1 over human CTPS2.

A cancer or cancer cells may have growth patterns of the cancer cells suggesting sensitivity to CTPS1 inhibition which is expected to be due to substantial loss of CTPS2 activity.

The CTPS1 inhibitor may be for administration to a subject identified as having a cancer expected to be susceptible to treatment by a CTPS1 inhibitor (e.g. the cancer is deficient in CTPS2). The CTPS1 inhibitor may be for administration to a subject from whom a sample of cancer cells has been shown to be susceptible to treatment by a CTPS1 inhibitor (e.g. the cancer is deficient in CTPS2).

Suitably, the cancer is selected from the group consisting of ovarian cancer, oesophageal cancer, bladder cancer, non-small cell lung cancer, gastric cancer, sarcoma, head and neck cancer, pancreatic adenocarcinoma, pancreatic neuroendocrine tumours, biliary tract cancer, melanoma, endometrial cancer, hepatocellular cancer, cervical cancer, bone cancer, central nervous system cancer, breast cancer, prostate cancer, colorectal cancer and renal cancer.

Suitably, the cancer is a cancer for which there is no other suitable therapy available.

Suitably, the cancer is selected from the group consisting of ovarian cancer, oesophageal cancer, bladder cancer, non-small cell lung cancer and gastric cancer.

Suitably, the cancer is ovarian cancer, such as serous ovarian cancer.

The cancer may be a lung cancer, such as non-small cell lung cancer.

The cancer may be gastrooesophageal cancer. The cancer may be oesophageal cancer. Alternatively, the cancer may be gastric cancer.

The cancer may be head and neck cancer.

The cancer may be bladder cancer.

The cancer may be sarcoma.

In one embodiment the cancer is a haematological cancer. In one embodiment the cancer is a non-haematological cancer. Suitably, the cancer is not a T-cell leukemia, such as T-cell acute lymphoblastic leukemia.

Suitably, the cancer or cancer cells are from a cancer type in which at least 10% are deficient in CTPS2, such as at least 15% of are deficient in CTPS2, especially as at least 18% are deficient in CTPS2. Deficiency in CTPS2 function may be due to homozygous deletion arising in females or hemizygous deletion arising in males.

Suitably, the cancer or cancer cells are from a cancer type in which at least 10% are deficient in CTPS2, such as at least 15% of are deficient in CTPS2, especially as at least 20% are deficient in CTPS2, in particular at least 40% are deficient in CTPS2. Deficiency in CTPS2 function may be determined by immunohistochemistry, such as absence of staining for CTPS2.

### Administration

The invention is typically intended for use with mammalian subjects, in particular human subjects. The CTPS1 inhibitor will typically be administered to a subject in need thereof, in particular a mammalian subject in need thereof, in particular a human subject in need thereof. A human subject may be an adult, such as aged 18 to 65. Alternatively, a human subject may be 66 years old or older. Alternatively, a human subject may be less than 18 years of age, such as 4 to 17 years old. A human subject may be male. Alternatively, a human subject may be female.

The CTPS2 gene is located on the human X chromosome and is not imprinted in human females, and thus the level of CTPS2 expression is expected to be higher in human female subjects (i.e. those with XX chromosomes) compared to human male subjects (i.e. those with XY chromosomes), as CTPS2 is expressed from each of the X chromosomes in human female subjects. In human female subjects, CTPS2 deficiency may result from genomic alteration(s) and/or epigenic change(s) in one or both XX chromosomes, suitably both XX chromosomes.

Suitably, when the human subject is male, deficiency in CTPS2 function may result from (i) complete loss of the CTPS2 gene by genomic deletion; (ii) partial loss of the CTPS2 gene by genomic deletion; (iii) disruption of the CTPS2 gene by a structural DNA variant such as an inversion, duplication or translocation falling within the footprint of the gene; (iv) mutation of the CTPS2 gene such that CTPS2 expression is substantially reduced, or completely lost; or (v) altered expression of the CTPS2 gene, for example due to changes in regulatory elements due to altered methylation and/or histone modification, such that CTPS2 expression is substantially reduced, or completely lost, particularly complete loss of the CTPS2 gene by genomic deletion. In particular, when the human subject is male, deficiency in CTPS2 function may result from the complete loss of the CTPS2 gene by genomic deletion.

Suitably, when the human subject is female (i.e. those with two X chromosomes), deficiency in CTPS2 function may result from complete loss of the CTPS2 gene on a first X chromosome by genomic deletion, and (i) complete loss of the CTPS2 gene on a second X chromosome by genomic deletion; (ii) partial loss of the CTPS2 gene on a second X chromosome by genomic deletion; (iii) disruption of the CTPS2 gene on a second X chromosome by a structural DNA variant such as an inversion, duplication or translocation falling within the footprint of the gene; (iv) mutation of the CTPS2 gene on a second X chromosome such that CTPS2 expression is substantially reduced, or completely lost; or (v) altered expression of the CTPS2 gene on a second X chromosome, for example due to changes in regulatory elements due to altered methylation and/or histone modification, such that CTPS2 expression is substantially reduced, or completely lost.

Suitably, when the human subject is female, deficiency in CTPS2 function may result from complete loss of the CTPS2 gene on a first X chromosome by genomic deletion and complete loss of the CTPS2 gene on a second X chromosome by genomic deletion.

Administration of the CTPS1 inhibitor The CTPS1 inhibitor may be administered by any suitable route, which may depend on the nature of the specific agent. Exemplary routes include oral, parenteral, buccal, sublingual, nasal or rectal administration. Conveniently, the CTPS1 inhibitor is administered orally.

The CTPS1 inhibitor may be provided in the form of a pharmaceutical composition comprising the CTPS1 inhibitor and a pharmaceutically acceptable carrier or excipient.

If delivered orally, the CTPS1 inhibitor may suitably be delivered in a solid pharmaceutical composition (such as a tablet, capsule or lozenge) or in a liquid pharmaceutical composition (such as a suspension, emulsion or solution).

A liquid formulation will generally consist of a suspension or solution of the CTPS1 inhibitor in a suitable liquid carrier e.g. an aqueous solvent such as water, ethanol or glycerine, or a non-aqueous solvent, such as polyethylene glycol or an oil. The formulation may also contain a suspending agent, preservative, flavouring and/or colouring agent.

A tablet formulation can be prepared using any suitable pharmaceutical carrier(s) routinely used for preparing solid formulations, such as magnesium stearate, starch, lactose, sucrose and cellulose.

Suitably, the pharmaceutical composition is in unit dose form, such as a tablet, capsule or ampoule. Suitably the unit dose form is for oral delivery.

The pharmaceutical composition may for example contain from 0.1% to 99.99% by weight, for example from 10 to 60% by weight, of the active material, depending on the method of administration. The pharmaceutical composition may contain from 0.01% to 99% by weight, for example 40% to 90% by weight, of the carrier, depending on the method of administration.

The pharmaceutical composition may contain from 0.05 mg to 2000 mg of the active material, for example from 1.0 mg to 500 mg, depending on the method of administration. The pharmaceutical composition may contain from 50 mg to 1000 mg of the carrier, for example from 100 mg to 400 mg, depending on the method of administration.

The dose of the compound used will vary in the usual way with the seriousness of the cancer, the weight of the sufferer, and other similar factors. However, as a general guide, suitable unit doses may be 0.05 mg to 1000 mg, more suitably 1.0 mg to 500 mg, and such unit doses may be administered more than once a day, for example two or three a day. Such therapy may extend for a number of weeks, months or longer. A plurality of unit does, such as a plurality of tablets, may be taken together.

Suitably, the CTPS1 inhibitor is administered orally, such as administered orally in a solid pharmaceutical composition.

The dose provided to a subject will typically be a safe and effective dose, i.e. an amount providing an acceptable balance of desired benefits and undesired side effects. A “safe and effective amount” is intended to include an amount of a compound that is effective to achieve a desirable effect in treatment of a disease-state. A desirable effect is typically clinically significant and/or measurable, for instance in the context of (a) inhibiting the disease-state, i.e., slowing or arresting its development; and/or (b) relieving the disease-state, i.e., causing regression of the disease state or a reduction in associated symptoms. The safe and effective amount is one that is sufficient to achieve the desirable effect when the CTPS1 inhibitor is administered.

For avoidance of doubt, a “safe and effective amount” as recited herein can be achieved by any suitable dosage regimen. Hence, for example, references herein to administering a safe and effective amount of a compound, such as by a particular administration route, include achieving the safe and effective amount via a single dose or by plural doses, such as administered by the specified administration route. For instance, orally administering a safe and effective amount includes both orally administering a single dose and orally administering any plural number of doses, provided that a safe and effective amount is thereby achieved by oral administration.

Administration of a CTPS1 inhibitor may typically be once or twice per day. Administration may be continuous, e.g. at least daily for a period of multiple weeks or discontinuous e.g. at least daily for one week, followed by a week without administration, followed at least daily for one further week.

Combinations with Further Agents

Treatment with the CTPS1 inhibitor may be combined with one or more further pharmaceutically acceptable active ingredients, which may be selected from: anti-mitotic agents such as vinblastine, paclitaxel and docetaxel; alkylating and DNA-cross linking agents, for example cisplatin, carboplatin, dacarbazine and cyclophosphamide; antimetabolites, for example 5-fluorouracil, cytosine arabinoside and hydroxyurea; intercalating agents for example adriamycin and bleomycin; topoisomerase inhibitors for example etoposide, topotecan and irinotecan; thymidylate synthase inhibitors for example raltitrexed; P13-kinase inhibitors for example idelalisib; MTOR inhibitors for example everolimus and temsirolimus; proteasome inhibitors for example bortezomib; histone deacetylase inhibitors for example panobinostat or vorinostat; hedgehog pathway blockers such as vismodegib; IAP inhibitors, such as LCL161; VEGF inhibitors such as bevacizumab; or pan-kinase inhibitors such as sorafenib and sunitinib The CTPS1 inhibitor and the additional pharmaceutically acceptable active ingredients may each be administered in any combination of separate, sequential or simultaneous dosing.

If administered simultaneously, the CTPS1 inhibitor may be e.g. (a) formulated separately from the further pharmaceutically acceptable active ingredient, or (b) formulated together with the further pharmaceutically acceptable active ingredient.

The further pharmaceutically acceptable active ingredient may be selected from tyrosine kinase inhibitors such as, for example, axitinib, dasatinib, erlotinib, imatinib, nilotinib, pazopanib and sunitinib. Alternatively, the further pharmaceutically acceptable active ingredient may be selected from azacitidine, decitabine, or cytarabine.

Further pharmaceutically acceptable active ingredients also include anticancer antibodies, such as those selected from the group consisting of anti-CD20 antibodies (such as obinutuzumab, ofatumumab, tositumomab or rituximab) or other antibodies such as olaratumab, daratumumab, necitumumab, dinutuximab, traztuzumab emtansine, pertuzumab, brentuximab, panitumumab, catumaxomab, bevacizumab, cetuximab, traztuzumab and gentuzumab ozogamycin.

The CTPS1 inhibitor may also be administered in combination with radiotherapy, surgery, hyperthermia therapy and/or cryotherapy.

In some embodiments the subject received chemotherapy, radiotherapy, surgery, hyperthermia therapy and/or cryotherapy prior to CTPS1 administration.

In some embodiments the subject concurrently receives chemotherapy, radiotherapy, surgery, hyperthermia therapy and/or cryotherapy with CTPS1 administration.

In some embodiments the subject subsequently receives chemotherapy, radiotherapy, surgery, hyperthermia therapy and/or cryotherapy after CTPS1 administration.

The invention is further exemplified by the following non-limiting examples.

### EXAMPLES

### Example 1: Human CTPS1 Enzyme Inhibition

The enzyme inhibitory activities of compounds against CTPS1 may be determined using the ADP-GIo™ Max assay (Promega, UK).

Assays for human CTPS1 were performed in 1× assay buffer containing 50 mM Tris, 10 mM MgCl₂, 0.01% Tween-20, pH to 8.0 accordingly. Finally, immediately before use, L-cysteine was added to the 1× assay buffer to a final concentration of 2 mM. All reagents are from Sigma-Aldrich unless specified otherwise. Human full length active C-terminal FLAG-Hiss-tag CTPS1 (UniProtKB-P17812, CTPS[1-591]-GGDYKDDDDKGGHHHHHHHH, SEQ ID NO: 1) was obtained from Proteros biostructures GmbH.

### Assay Procedure

3× human CTPS1 protein was prepared in 1× assay buffer to the final working protein concentration required for the reaction. A 2 uL volume per well of 3× human CTPS1 protein was mixed with 2 uL per well of 3× test compound (compound prepared in 1× assay buffer to an appropriate final 3× compound concentration respective to the concentration response curve designed for the compounds under test) for 10 minutes at 25° C. The enzymatic reaction was then initiated by addition of a 2 uL per well volume of a pre-mixed substrate mix (UltraPure ATP from ADP-GIo™ Max kit (0.31 mM), GTP (0.034 mM), UTP (0.48 mM) and L-glutamine (0.186 mM)) and the mixture was incubated for an appropriate amount of time within the determined linear phase of the reaction at 25° C. under sealed plate conditions with constant agitation at 500 revolutions per minute (rpm). ADP-GIo™ Max reagent was added for 60 minutes (6 μL per well) and subsequently ADP-GIo™ Max development reagent was added for 60 minutes (12 uL per well) prior to signal detection in a microplate reader (EnVision® Multilabel Reader, Perkin Elmer). Following each reagent addition over the course of the assay, assay plates were pulse centrifuged for 30 seconds at 500 rpm.

In all cases, the enzyme converts ATP to ADP and the ADP-Glo™ Max reagent subsequently depletes any remaining endogenous ATP in the reaction system. The ADP-Glo™ Max detection reagent converts the ADP that has been enzymatically produced back into ATP and using ATP as a substrate together with luciferin for the enzyme luciferase, light is generated which produces a detectable luminescence. The luminescent signal measured is directly proportional to the amount of ADP produced by the enzyme reaction and a reduction in this signal upon compound treatment demonstrates enzyme inhibition. The percentage inhibition produced by each concentration of compound was calculated using the equation shown below:

$$\% Inhibition = 1 - \frac{( {Mean}_{Min} - {Mean}_{Inh} )}{( {Mean}_{Min} - {Mean}_{Max} )} \times 100$$

Percentage inhibition was then plotted against compound concentration, and the 50% inhibitory concentration (IC₅₀) was determined from the resultant concentration-response curve.

### Example 2: RapidFire/MS-based CTPS1 Enzyme Selectivity Assays

Human CTPS1 versus CTPS2 Selectivity Assessment by RapidFire/MS Analysis.

The enzyme inhibitory activities against each target isoform of interest were determined for compounds using an optimised RapidFire high-throughput mass spectrometry (RF/MS) assay format. RF/MS assays for both human CTPS1 and CTPS2 were performed in assay buffer consisting of 50 mM HEPES (Merck), 20 mM MgCl₂, 5 mM KCl, 1 mM DTT, 0.01% Tween-20, pH to 8.0 accordingly. Human full-length active C-terminal FLAG-His-tag CTPS1 (UniProtKB-P17812, CTPS[1-591]-GGDYKDDDDKGGHHHHHHHH, SEQ ID NO: 1) was obtained from Proteros biostructures GmbH. Human full length active C-terminal FLAG-His-Avi tagged CTPS2 (UniProtKB-Q9NRF8, CTPS2 [1-586]-DYKDDDDKHHHHHHGLNDIFEAQKIEWHE, SEQ ID NO: 2) was obtained from Harker Bio.

### Assay Procedure

Human CTPS (1 or 2) protein was prepared in 1× assay buffer to the final working protein concentration required for the reaction. A 2 uL volume per well of 2×CTPS (1 or 2) protein was mixed with 40 nL of compound using acoustic (ECHO) delivery and incubated for 10 minutes at 25° C. Each isoform enzymatic reaction was subsequently initiated by addition of 2 uL per well of a 2× substrate mix in assay buffer. For hCTPS1; ATP (0.3 mM), UTP (0.2 mM), GTP (0.07 mM) and L-glutamine (0.1 mM). For hCTPS2; ATP (0.1 mM), UTP (0.04 mM), GTP (0.03 mM) and L-glutamine (0.1 mM). Each mixture was incubated for an appropriate amount of time per isoform within the determined linear phase of the reaction at 25° C. A 60 uL volume of stop solution (1% formic acid with 0.5 uM ¹³C₉—¹⁵N₃—CTP in H₂O) was added and the plate immediately heat-sealed and centrifuged for 10 minutes at 4,000 rpm. Following centrifugation, plates were loaded onto the Agilent RapidFire microfluidic solid phase extraction system coupled to an API4000 triple quadrupole mass spectrometer (RF/MS) for analysis.

In all cases, the enzyme converts UTP to CTP. Highly specific and sensitive multiple reaction monitoring (MRM) MS methods may be optimised for the detection of the enzymatic reaction product, CTP, and the stable isotope labelled product standard ¹³C₉—¹⁵N₃-CTP. Readout for data analysis was calculated as the ratio between the peak area of the product CTP and the internal standard ¹³C₉—¹⁵N₃-CTP. For data reporting, the following equation was used:

$$R = \frac{P}{IS}$$

(R=ratio/readout, P=product signal area, IS=internal standard signal area)

For each screening plate, the means of the negative (DMSO) and positive control values were used for the calculation of the respective assay window (S/B) and Z′ values. The median of the respective control values was used for calculation of percent inhibition according to the following equation:

$$I = \frac{{R}_{neg} - {R}_{sample} \%}{[ {R}_{neg} - {R}_{pos} ]}$$

(I=Inhibition, R_{neg}=median of negative control readout values, Rₚₒₛ=median of positive control readout values, Rₛₐₘₚₗₑ=sample readout value) Percentage inhibition was then plotted against compound concentration, and the 50% inhibitory concentration (IC₅₀) was determined from the resultant concentration-response curve.

Fold selectivity between CTPS1 and CTPS2 was subsequently calculated according to the following equation:

$$Fold selectivity = \frac{CTPS 2 {IC}_{50}}{CTPS 1 {IC}_{50}}$$

### Example 3: Cells Require CTPS Enzymatic Activity in Order to Proliferate

In vitro cell proliferation assays have been used to establish an essential role for CTP synthase activity, and to assess the differential activity of the CTPS1 and CTPS2 isoforms.

Cells engineered to express neither CTPS isoform (i.e. lacking CTPS1 and CTPS2) undergo rapid cell death by apoptosis (Martin 2014, Martin 2020, Minet 2022).

Human embryonic kidney (HEK) cells lacking either CTPS1 or CTPS2 were generated using CRISPR technology, with lack of expression of the relevant protein confirmed by western blot (Minet 2022). Cells were cultured at 37° C. with 5% CO₂; viable cells were enumerated using the CellTiter-Glo 2.0 reagent (Promega). HEK cells expressing only CTPS2 proliferated at a slower rate than HEK cells expressing only CTPS1, implying greater enzymatic activity for the CTPS1 isoform (Minet 2022). This is consistent with recent work also demonstrating that the CTPS1 isoform has higher enzymatic activity than CTPS2 (Lynch 2021).

Together, these findings indicate that CTP synthase activity is a requirement for cell survival, and indicate that the CTPS1 isoform has higher enzymatic activity than CTPS2.

### Example 4: CTPS1 Involvement in the Proliferation of Cancer Cells

Pathways involved in providing the key building blocks for nucleic acid replication are the purine and pyrimidine synthesis pathways, and pyrimidine biosynthesis has been observed to be up-regulated in tumors and neoplastic cells. CTPS activity is upregulated in a range of tumour types of both haematological and non-haematological origin, although heterogeneity is observed among patients. Linkages have also been made between high enzyme levels and resistance to chemotherapeutic agents.

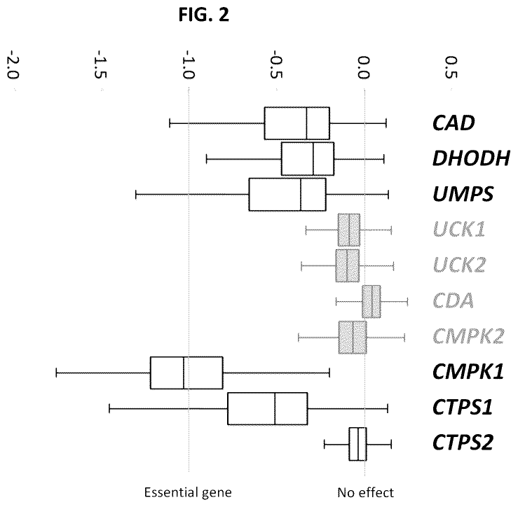

In an analysis of published data, CTPS1 was found by the present inventors to be essential for the proliferation of human cancer cells derived from a broad range of haematological and solid tumour types, whereas CTPS2 was invariably redundant. This analysis used data from the Achilles project where every gene in the human genome was independently deleted using CRIPR technology in each of 324 human cancer cell lines, and the effects of each gene deletion was assessed using an in vitro proliferation assay (Behan 2019). This dataset has subsequently been expanded to include data from 1,032 human cancer cell lines (Cancer Dependency Map:p.org/). The effects of deletion of different genes in the pyrimidine synthesis pathway were assessed (see FIG. 2). Deletion of CTPS2 had no effect on cancer cell proliferation. Deletion of genes in the salvage pathway (CDA, UCK1, UCK2 or CMPK2) had minimal effect on cell proliferation. Deletion of CMPK1 had a marked effect on cell proliferation, consistent with CMPK1 being an essential gene. Deletion of CTPS1, UMPS, DHODH or CAD inhibited cancer cell proliferation with an effect that is consistent with dependency of cancer cells on the products of these genes; inhibition of CTPS1 produced the greatest impairment of cancer cell proliferation. These findings indicate that the majority of cancer cells are dependent on CTPS1 for cell proliferation, likely due to its enhanced enzymatic activity, whereas CTPS2 is not required.

### Example 5: CTPS2 is Lost in a Subset of Cancers

Studies outlined above indicate an essential role for CTP synthase activity, with lack of CTP synthase resulting in cell death by apoptosis. Studies also indicate that many cancer cells are dependent on the activity of CTPS1, the more active of the two CTPS isoforms. Cancer cells that are less sensitive to loss or inhibition of CTPS1 may be able to utilise CTPS2 activity in order to survive and proliferate. Consistent with this notion, the expression of CTPS2 can be increased in certain physiological situations (Martin 2014), raising the further possibility that increased CTPS2 expression may be associated with acquired resistance to therapy with a CTPS1 inhibitor. Cancer cells lacking CTPS2 expression due to genetic deletion of the CTPS2 gene could therefore be highly sensitive to CTPS1 inhibition. It also follows from the studies outlined above that genetic deletion of CTPS2 would not in itself be deleterious to cancer cells due to the continued availability of CTPS1 enzymatic activity.

Loss of genetic material, due to genomic deletions, is a common phenomenon in cancer and may confer a selective advantage due to deletion of tumour suppressor genes. It has also been recognised that such genomic deletions may result in bystander loss of genes that do not confer a selective advantage to the cancer cells; this collateral damage may, however, expose therapeutic vulnerabilities in the cancer cells (Achreja 2022).

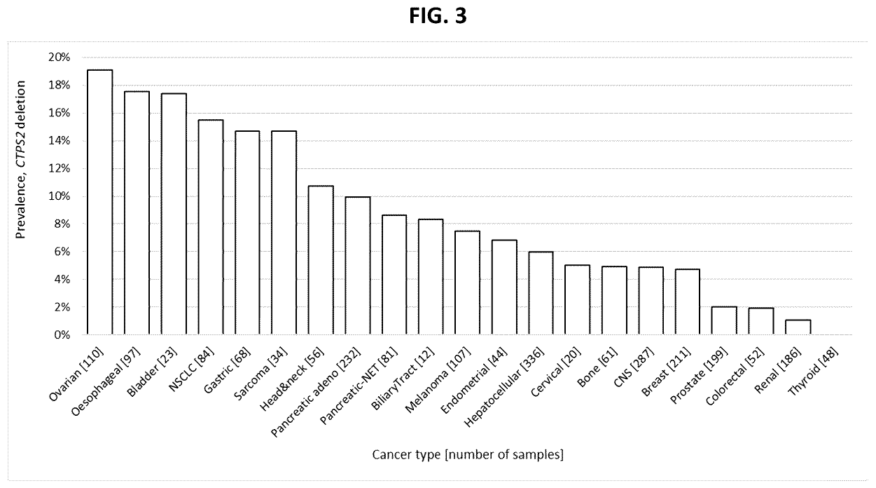

In an analysis of publicly available data (ICGC 2020), CTPS2 was found to be deleted in a subset of cancer samples, with the highest prevalence seen in ovarian, oesophageal and bladder cancers (see FIG. 3). These findings identify a mechanism to identify patients with cancers that are predicted to be highly sensitive to therapy with a CTPS1 inhibitor.

### Example 6: Level of CTPS2 expression predicts cellular response to CTPS2 loss

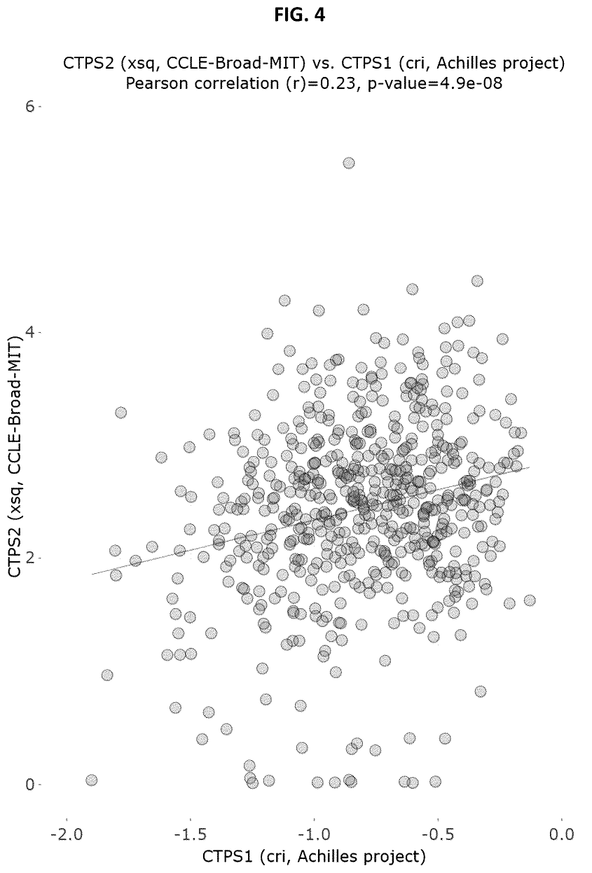

In an analysis of publicly available data, CTPS2 expression levels were found by the present inventors to be correlated with sensitivity to CTPS1 loss by CRISPR knockout. In this analysis, which combined data from the Achilles project and the Cancer Cell Line Encyclopedia for 573 human cancer cells lines, lower CTPS2 expression (measured by RNA abundance) was associated with increased sensitivity to CTPS1 loss by CRISPR (FIG. 4, Pearson correlation r=0.23, P-value=4.9×10⁻⁸). No association was found between CTPS1 expression and sensitivity to CTPS1 loss by CRISPR (P-value=0.075).

CTPS2 expression levels were also found to correlate with IC₅₀ values for CTPS-IA. Human cancer cells lines were cultured in triplicate at 37° C. with 5% CO₂ in the presence of different concentrations of CTPS-IA or in the presence of vehicle alone and viable cells were enumerated after 72 hours using the CellTiter-Glo 2.0 reagent (Promega) or a comparable reagent. A nonlinear regression model was used to calculate half maximal inhibitory concentration (IC₅₀). CTPS1 and CTPS2 expression levels, measured by RNA abundance, were either generated by RNA sequencing or retrieved from the publicly available Cancer Cell Line Encyclopedia. In a set of 19 human cancer cell lines derived from haematological cancers, lower CTPS2 expression was associated with increased sensitivity to CTPS-IA (Pearson correlation r=0.50, P-value=0.0007). In a set of 60 human cancer cell lines covering common cancer types, lower CTPS2 expression was associated with increased sensitivity to CTPS-IA (Pearson correlation r=0.24, P-value=<0.0001). In an independent set of 103 human cancer cell lines covering common cancer types, lower CTPS2 expression was associated with increased sensitivity to CTPS-IA (Pearson correlation r=0.14, P-value=<0.0001). The level of CTPS1 expression was not associated with sensitivity to CTPS-IA in any of these three experiments (P-value>0.05 in all cases).

Together, these data indicated that the level of expression of CTPS2, but not CPTS1, is associated with sensitivity to CTPS1 loss or inhibition, by CRISPR or by pharmacological inhibition, respectively. Cancer cell lines with low expression of CTPS2 show enhanced sensitivity to CTPS1 loss or inhibition.

### Example 7: CTPS2 protein is absent in a subset of human cancers

Primary human cancer samples were analysed for CTPS2 protein expression by immunohistochemistry. A CTPS2 monoclonal antibody was validated using CTPS1 and CTPS2 knockout cell lines and normal human tissues. Having demonstrated selective staining of CTPS2 in these experiments, the antibody was used to examine tissue microarrays of primary human cancer samples. CTPS2 negative samples are defined by the absence of staining for CTPS2 (an isotype control antibody was used to detect non-specific staining).

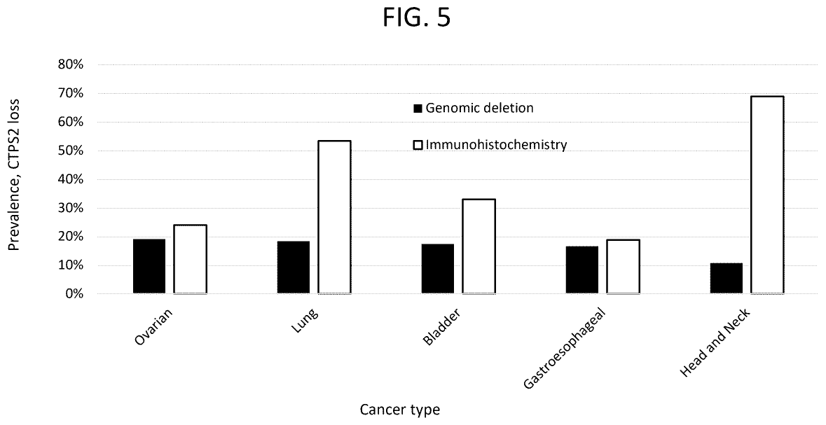

Analysis of samples from 117 patients diagnosed with high grade serous ovarian cancer identified a prevalence of 24% for tumours showing loss of CTPS2 protein expression by immunohistochemistry. Analysis of samples from 80 patients diagnosed with gastroesophageal adenocarcinoma identified a prevalence of 18% for tumours showing loss of CTPS2 protein expression by immunohistochemistry. For both of these cancer types, the prevalence of CTPS2 loss by genomic studies is well correlated with the prevalence of CTPS2 loss as assessed by immunohistochemistry (FIG. 5).

Analysis of samples from 208 patients diagnosed with lung cancer identified a prevalence of 65% for tumours showing loss of CTPS2 protein expression by immunohistochemistry. Analysis of samples from 208 patients diagnosed with bladder cancer identified a prevalence of 37% for tumours showing loss of CTPS2 protein expression by immunohistochemistry. Analysis of samples from 208 patients diagnosed with head and neck squamous cell cancer identified a prevalence of 66% for tumours showing loss of CTPS2 protein expression by immunohistochemistry. For these three cancer types, whilst both immunohistochemistry and genomic studies confirmed that a notable proportion of cancers are deficient in CTPS2, the prevalence of CTPS2 loss assessed by immunohistochemistry was higher than the prevalence of CTPS2 loss observed in genomic studies (FIG. 5). Possible explanations for this discrepancy include partial gene deletion of CTPS2 that is not detected by genomic studies but results in loss of protein expression; differences in the stage of the cancers in the cohorts assessed by genomics and immunohistochemistry (for example, cohorts of samples undergoing whole genome sequencing are often biased towards patients with early stage disease treated by surgical resection where sufficient material for sequencing is generally available); or loss of protein expression due to epigenetic regulation such as gene methylation.

### Example 8: Further Characterisation of the Prevalence of Reduced or Absent CTPS2 Expression in Target Cancers

Further characterisation of the prevalence of reduced or absent CTPS2 expression could be undertaken in a target cancer, for example ovarian cancer, through the analysis of tissue samples from multiple patient tumours, for example using a tumour microarray, using a suitably qualified assay for the detection of CTPS2 expression, for example an immunohistochemistry assay.

Further characterisation of the cellular consequences of loss of CTPS2 expression could be examined through the generation of pairs of isogenic human cancer cells lines where CTPS2 expression is abrogated in a subclone of cells, for example using CRISPR (clustered regularly interspaced short palindromic repeats) technology to disrupt the CTPS2 gene followed by a comparison of key cellular characteristics (including proliferation and apoptosis in the presence or absence of a CTPS2 inhibitor) between isogenic cells with and without expression of CTPS2.

Further information could be obtained by assessing pairs of isogenic human cancer cell lines with and without expression of CTPS2 in an in vivo transplantation model, for example transplantation into an immunocompromise mouse, where the affects of treatment with a CTPS1 inhibitor are ascertained, for example by measuring and comparing the growth kinetics of isogenic human cancer cell lines with and without expression of CTPS2 when the transplanted mice are treated with a CTPS1 inhibitor.

Further characterisation of the cellular consequences of loss of CTPS2 expression could be examined through the comparison of primary patient derived tumour samples that either express CTPS2 protein or have lost expression of CTPS2 protein, for example by analysing primary tumour samples for CTPS2 protein expression by immunohistochemistry and comparing key cellular characteristics of CTPS2 expressing and CTPS2 null tumours, including proliferation and apoptosis, in the presence or absence of a CTPS1 inhibitor in vitro, or assessing primary tumour samples with and without expression of CTPS2 in an in vivo transplantation model, for example transplantation into an immunocompromise mouse, where the effects of treatment with a CTPS1 inhibitor are ascertained, for example by measuring and comparing the growth kinetics of primary human cancers with and without expression of CTPS2 when the transplanted mice are treated with a CTPS1 inhibitor.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the claims which follow.

All publications, including but not limited to patents and patent applications, cited in this specification are herein incorporated by reference as if each individual publication were specifically and individually indicated to be incorporated by reference herein as though fully set forth.

### Clauses of the Invention:

A series of clauses setting out embodiments of the invention are as follows.
Clause 1. A method for the treatment of CTPS2 deficient cancer in a subject, the method comprising administering a CTPS1 inhibitor to the subject.
Clause 2. A method for the treatment of cancer in a subject, the method comprising the steps of:
   i) identifying that the subject has a cancer which is deficient in CTPS2; and
   ii) administering a CTPS1 inhibitor to the subject.
Clause 3. A method for the treatment of cancer in a subject, the method comprising the steps of:
   i) providing a sample from the subject;
   ii) identifying that a subject has a cancer which is deficient in CTPS2; and
   iii) administering a CTPS1 inhibitor to the subject.
Clause 4. A method for the treatment of cancer in a subject, the method comprising the steps of:
   i) obtaining a sample from the subject;
   ii) identifying that a subject has a cancer which is deficient in CTPS2; and
   iii) administering a CTPS1 inhibitor to the subject.
Clause 5. A method for the treatment of cancer which may be susceptible to treatment with a CTPS1 inhibitor in a subject, the method comprising the steps of:
   i) identifying that the cancer is deficient in CTPS2; and
   ii) administering a CTPS1 inhibitor.
Clause 6. A method for the treatment of cancer which may be susceptible to treatment with a CTPS1 inhibitor in a subject, the method comprising the steps of:
   i) providing a sample from the subject;
   ii) identifying that the cancer is deficient in CTPS2; and
   iii) administering a CTPS1 inhibitor.
Clause 7. A method for the treatment of cancer which may be susceptible to treatment with a CTPS1 inhibitor in a subject, the method comprising the steps of:
   i) obtaining a sample from the subject;
   ii) identifying that the cancer is deficient in CTPS2; and
   iii) administering a CTPS1 inhibitor.
Clause 8. A method of determining that cancer cells may be susceptible to treatment with a CTPS1 inhibitor, the method comprising the step of identifying that the cancer cells are deficient in CTPS2.
Clause 9. A method of determining that a cancer in a subject may be susceptible to treatment with a CTPS1 inhibitor, the method comprising the step of identifying that the cancer is deficient in CTPS2.
Clause 10. A CTPS1 inhibitor for use in the treatment of CTPS2 deficient cancer.
Clause 11. A pharmaceutical composition comprising a CTPS1 inhibitor and a pharmaceutically acceptable excipient or carrier for use in the treatment of CTPS2 deficient cancer.
Clause 12. Use of a CTPS1 inhibitor in the manufacture of a medicament for the treatment of cancer CTPS2 deficient cancer.
Clause 13. The method, CTPS1 inhibitor for use, pharmaceutical composition for use or use according to any one of clauses 1 to 12, wherein the CTPS2 deficient cancer is selected from the group consisting of ovarian cancer, oesophageal cancer, bladder cancer, non-small cell lung cancer, gastric cancer, sarcoma, head and neck cancer, pancreatic adenocarcinoma, pancreatic neuroendocrine tumours, biliary tract cancer, melanoma, endometrial cancer, hepatocellular cancer, cervical cancer, bone cancer, central nervous system cancer, breast cancer, prostate cancer, colorectal cancer and renal cancer.
Clause 14. The method, CTPS1 inhibitor for use, pharmaceutical composition for use or use according to clause 13, wherein the CTPS2 deficient cancer is selected from the group consisting of ovarian cancer, oesophageal cancer, bladder cancer, non-small cell lung cancer and gastric cancer.
Clause 15. The method, CTPS1 inhibitor for use, pharmaceutical composition for use or use according to any one of clauses 1 to 12, wherein the CTPS2 deficient cancer is ovarian cancer.
Clause 16. The method, CTPS1 inhibitor for use, pharmaceutical composition for use or use according to clause 15, wherein the CTPS2 deficient cancer is serous ovarian cancer.
Clause 17. The method, CTPS1 inhibitor for use, pharmaceutical composition for use or use according to any one of clauses 1 to 12, wherein the CTPS2 deficient cancer is lung cancer.
Clause 18. The method, CTPS1 inhibitor for use, pharmaceutical composition for use or use according to clause 17, wherein the CTPS2 deficient cancer is non-small cell lung cancer.
Clause 19. The method, CTPS1 inhibitor for use, pharmaceutical composition for use or use according to any one of clauses 1 to 12, wherein the CTPS2 deficient cancer is oesophageal cancer.
Clause 20. The method, CTPS1 inhibitor for use, pharmaceutical composition for use or use according to any one of clauses 1 to 12, wherein the CTPS2 deficient cancer is gastric cancer.
Clause 21. The method, CTPS1 inhibitor for use, pharmaceutical composition for use or use according to any one of clauses 1 to 12, wherein the CTPS2 deficient cancer is head and neck cancer.
Clause 22. The method, CTPS1 inhibitor for use, pharmaceutical composition for use or use according to any one of clauses 1 to 12, wherein the CTPS2 deficient cancer is bladder cancer.
Clause 23. The method, CTPS1 inhibitor for use, pharmaceutical composition for use or use according to any one of clauses 1 to 12, wherein the CTPS2 deficient cancer is sarcoma.
Clause 24. The method, CTPS1 inhibitor for use, pharmaceutical composition for use or use according to any one of clauses 1 to 12, wherein the cancer is a haematological cancer.
Clause 25. The method, CTPS1 inhibitor for use, pharmaceutical composition for use or use according to any one of clauses 1 to 12, wherein the cancer is a non-haematological cancer Clause 26. The method, CTPS1 inhibitor for use, pharmaceutical composition for use or use according to any one of clauses 1 to 25, wherein the cancer is not a T-cell leukemia, such as T-cell acute lymphoblastic leukemia.
Clause 27. The method, CTPS1 inhibitor for use, pharmaceutical composition for use or use according to any one of clauses 1 to 26, wherein the CTPS1 inhibitor has an IC₅₀ of 10 uM or lower in respect of human CTPS1 enzyme.
Clause 28. The method, CTPS1 inhibitor for use, pharmaceutical composition for use or use according to clause 27, wherein the CTPS1 inhibitor has an IC₅₀ of 1 uM or lower in respect of human CTPS1 enzyme.
Clause 29. The method, CTPS1 inhibitor for use, pharmaceutical composition for use or use according to clause 28, wherein the CTPS1 inhibitor has an IC₅₀ of 100 nM or lower in respect of human CTPS1 enzyme.
Clause 30. The method, CTPS1 inhibitor for use, pharmaceutical composition for use or use according to any one of clauses 27 to 29, wherein the IC₅₀ of the CTPS1 inhibitor is established using the assay procedure set out in Example 1.
Clause 31. The method, CTPS1 inhibitor for use, pharmaceutical composition for use or use according to any one of clauses 1 to 30, wherein the CTPS1 inhibitor has a selectivity for human CTPS1 over human CTPS2 of at least 2-fold.
Clause 32. The method, CTPS1 inhibitor for use, pharmaceutical composition for use or use according to clause 31, wherein the CTPS1 inhibitor has a selectivity for human CTPS1 over human CTPS2 of at least 30-fold.
Clause 33. The method, CTPS1 inhibitor for use, pharmaceutical composition for use or use according to clause 32 wherein the CTPS1 inhibitor has a selectivity for human CTPS1 over human CTPS2 of at least 60-fold, such as at least 1000-fold.
Clause 34. The method, CTPS1 inhibitor for use, pharmaceutical composition for use or use according to any one of clauses 31 to 33, wherein the selectivity of the CTPS1 inhibitor is established using the assay procedure set out in Example 2.
Clause 35. The method, CTPS1 inhibitor for use, pharmaceutical composition for use or use according to any one of clauses 1 to 34, wherein the CTPS1 inhibitor is a compound of formula (I)

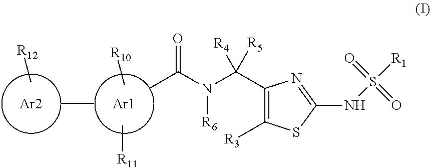

wherein
R₁ is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R₃ is H, CH₃, halo, OC₁₋₂alkyl or CF₃;
R₄ and R₅ are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl,
or R₄ and R₅ together with the carbon atom to which they are attached form a C₃₋₆cycloalkyl or C₃₋₆heterocycloalkyl ring;
R₆ is H or C₁₋₃alkyl;
Ar1 is a 6-membered aryl or heteroaryl;
Ar2 is a 6-membered aryl or heteroaryl and is attached to Ar1 in the para position relative to the amide;
R₁₀ is H, halo, C₁₋₃alkyl, OC₁₋₂alkyl, C₁₋₂haloalkyl, OC₁₋₂haloalkyl or CN; R₁₁ is H, F, Cl, CH₃, ethyl, OCH₃, CF₃, OCF₃ or CN;
R₁₂ is attached to Ar2 in the meta or ortho position relative to Ar1 and R₁₂ is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR₂₃R₂₄, SO₂CH₃, C(O)N(CH₃)₂, NHC(O)C₁₋₃alkyl, or a C₃₋₆heterocycloalkyl comprising one nitrogen located at the point of attachment to Ar2, or R₁₂ together with a nitrogen atom to which it is attached forms an N-oxide (N⁺—O—);
R₂₃ is H or C₁₋₂alkyl;
R₂₄ is H or C₁₋₂alkyl;
or a pharmaceutically acceptable salt and/or pharmaceutically acceptable solvate thereof.
Clause 36. The method, CTPS1 inhibitor for use, pharmaceutical composition for use or use according to clause 35, wherein the CTPS1 inhibitor is selected from the compounds disclosed in List A or a pharmaceutically acceptable salt and/or pharmaceutically acceptable solvate thereof.
Clause 37. The method, CTPS1 inhibitor for use, pharmaceutical composition for use or use according to any one of clauses 1 to 34, wherein the CTPS1 inhibitor is a compound of formula (II):

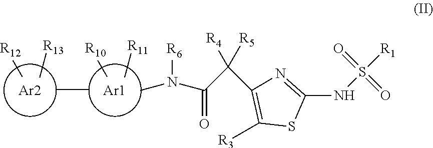

wherein
R₁ is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R₃ is H, halo, CH₃, OC₁₋₂alkyl or CF₃;
or R₃ together with R₅ forms a 5- or 6-membered cycloalkyl or 5 or 6 membered oxygen-containing heterocycloalkyl;
R₄ and R₅ are each independently H, halo, C₁₋₆alkyl, C₀₋₂alkyleneC₃₋₆cycloalkyl, C₀₋₂alkyleneC₃₋₆heterocycloalkyl, OC₁₋₆alkyl, OC₀₋₂alkyleneC₃₋₆cycloalkyl, C₁₋₃alkyleneOC₁₋₃alkyl, C₁₋₆alkylOH, C₁₋₆haloalkyl, OC₁₋₆haloalkyl or NR₂₁R₂₂, or R₄ is H and R₅ together with R₃ form a 5- or 6-membered cycloalkyl or 5 or 6 membered oxygen-containing heterocycloalkyl,
or R₄ and R₅ together with the carbon atom to which they are attached form a C₃₋₆cycloalkyl or C₃₋₆heterocycloalkyl,
or R₄ is H and R₅ and R₆ are a C₂₋₃alkylene chain forming a 5- or 6-membered ring;
or R₄ is O and R₅ is absent;
R₆ is H or C₁₋₃alkyl,
or R₆ together with R₁₁ when in the ortho-position to the amide are a C₂alkylene chain forming a 5-membered ring,
or R₅ and R₆ are a C₂₋₃alkylene chain forming a 5- or 6-membered ring and R₄ is H;
Ar1 is 6-membered aryl or heteroaryl;
Ar2 is a 6-membered aryl or heteroaryl and is attached to Ar1 in the para position relative to the amide;
R₁₀ is H, halo, C₁₋₃alkyl, OC₁₋₂alkyl, C₁₋₂haloalkyl, OC₁₋₂haloalkyl or CN;
R₁₁ is H, F, Cl, CH₃, ethyl, OCH₃, CF₃, OCF₃ or CN,
or R₁₁, when in the ortho-position to the amide, together with R₆ are a C₂alkylene chain forming a 5-membered ring;
R₁₂ is attached to Ar2 in the ortho or meta position relative to Ar1 and R₁₂ is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR₂₃R₂₄, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2, or R₁₂ together with a nitrogen atom to which it is attached forms an N-oxide (N^{+—O—);}
R₁₃ is H, halo, CH₃ or OCH₃;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
or a pharmaceutically acceptable salt and/or pharmaceutically acceptable solvate thereof.
Clause 38. The method, CTPS1 inhibitor for use, pharmaceutical composition for use or use according to clause 37, wherein the CTPS1 inhibitor is selected from the compounds disclosed in List B or a pharmaceutically acceptable salt and/or pharmaceutically acceptable solvate thereof.
Clause 39. The method, CTPS1 inhibitor for use, pharmaceutical composition for use or use according to any one of clauses 1 to 34, wherein the CTPS1 inhibitor is a compound of formula (III):

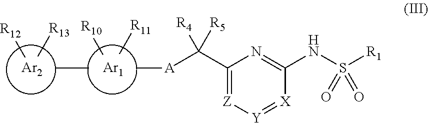

wherein
A is an amide linker having the following structure: —C(═O)NH— or —NHC(═O)—;
X is N or CH;
Y is N or CR₂;
Z is N or CR₃,
   with the proviso that when at least one of X or Z is N, Y cannot be N;
R₁ is C₁₋₅alkyl, C₀₋₂alkyleneC₃₋₅cycloalkyl which cycloalkyl is optionally substituted by CH₃, or CF₃;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
wherein at least one of R₂ and R₃ is H;
R₄ and R₅ are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R₄ and R₅ together with the carbon atom to which they are attached form a C₃₋₆cycloalkyl or C₃₋₆heterocycloalkyl; and
when A is —NHC(═O)—:
R₄ and R₅ may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to the amide;
R₁₀ is H, halo, C₁₋₃alkyl, C₁₋₂haloalkyl, OC₁₋₂alkyl, OC₁₋₂haloalkyl or CN; R₁₁ is H, F, C, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and when A is —NHC(═O)—:
R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O—);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
or a pharmaceutically acceptable salt and/or pharmaceutically acceptable solvate thereof.
Clause 40. The method, CTPS1 inhibitor for use, pharmaceutical composition for use or use according to clause 37, wherein the CTPS1 inhibitor is selected from the compounds disclosed in List C or a pharmaceutically acceptable salt and/or pharmaceutically acceptable solvate thereof.
Clause 41. The method, CTPS1 inhibitor for use, pharmaceutical composition for use or use according to clause 37, wherein the CTPS1 inhibitor is a compound of formula (IV):

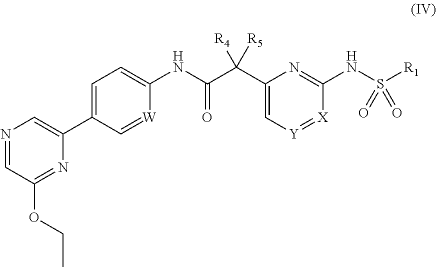

wherein:
(a) when R₄, R₅, X, Y and R₁ are as follows:

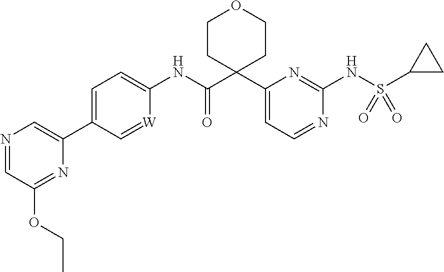

then W is N, CH or CF;
(b) when R₄, R₅, X, W and R₁ are as follows:

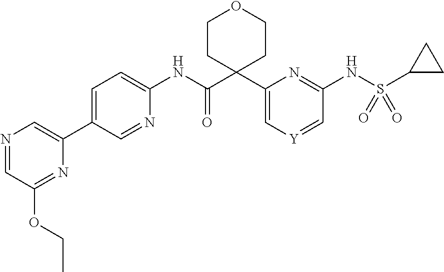

then Y is CH or N;
(c) when W, X, Y and R₁ are as follows:

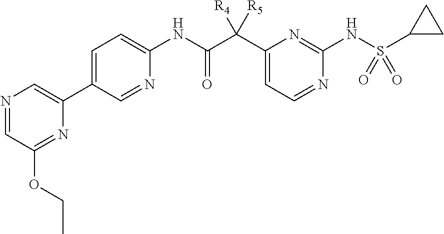

then R₄ and R₅ are joined to form the following structures:

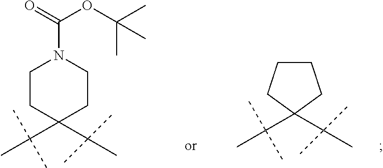

(d) when W, R₄, R₅, X and Y are as follows:

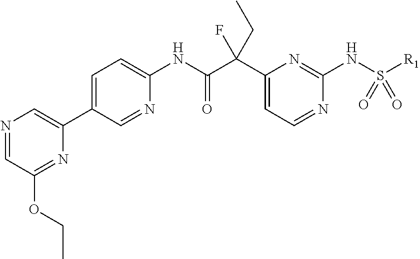

then R₁ is methyl or cyclopropyl; and
(e) the compound is selected from the group consisting of:

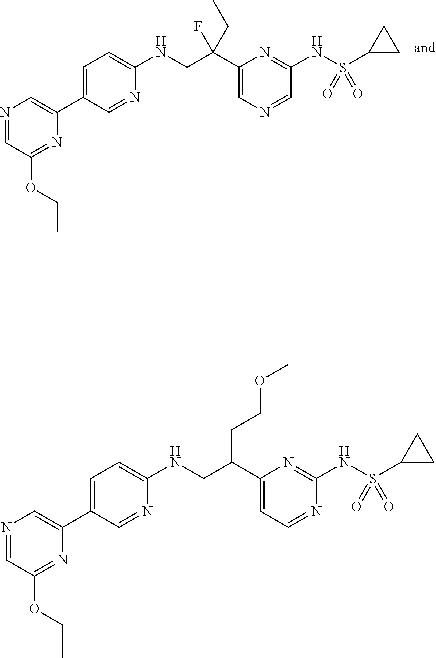 [SMILES: CCOc1cncc(-c2ccc(NCC(F)(CC)c3cncc(NS(=O)(=O)C4CC4)n3)nc2)n1 ; CCOc1cncc(-c2ccc(NCC(CCOC)c3ccnc(NS(=O)(=O)C4CC4)n3)nc2)n1]

or a pharmaceutically acceptable salt and/or pharmaceutically acceptable solvate thereof.
Clause 42. The method, CTPS1 inhibitor for use, pharmaceutical composition for use or use according to clause 41, wherein the CTPS1 inhibitor is selected from the compounds disclosed in List D or a pharmaceutically acceptable salt and/or pharmaceutically acceptable solvate thereof.
Clause 43. The method, CTPS1 inhibitor for use, pharmaceutical composition for use or use according to clause 39, wherein the CTPS1 inhibitor is a compound of formula (V):

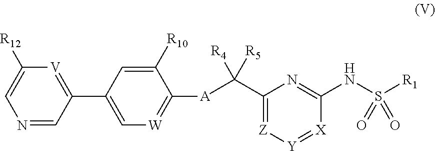

(a) when A, V, W, X, Y, Z, R₁, R₁₀ and R₁₂ are as follows:

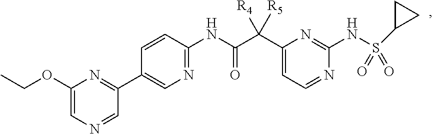

then R₄ and R₅ together with the carbon atom to which they attached form:

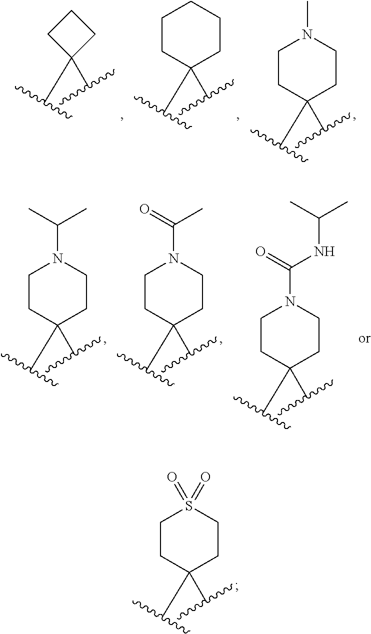

or
(b) when A, V, W, X, Y, Z, R₁, R₁₀ and R₁₂ are as follows:

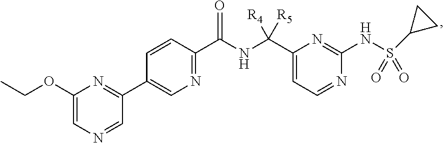

then R₄ and R₅ together with the carbon atom to which they are attached form:

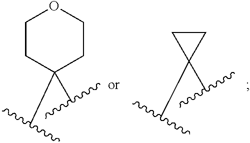 [SMILES: *C1(*)CCOCC1 ; *C1(*)CC1]

or
(c) when A, V, W, X, Y, Z, R₄, R₅, R₁₀ and R₁₂ are as follows:

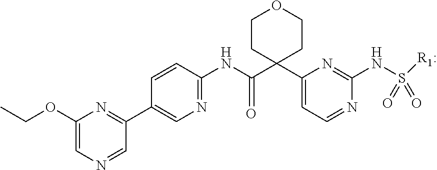

then R₁ is

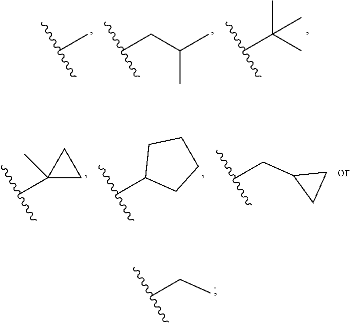

or
(d) when A, V, W, X, Y, Z, R₄, R₅, R₁₀ and R₁₂ are as follows:

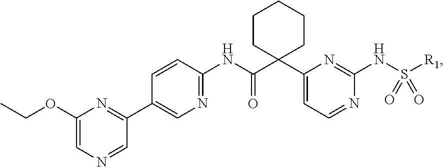

then R₁ is;

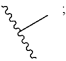

or
(e) when A, X, Y, Z, R₁, R₄ and R₅ are as follows:

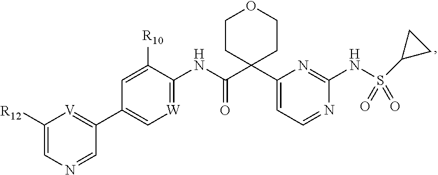

then V, W, R₁₀ and R₁₂ are:

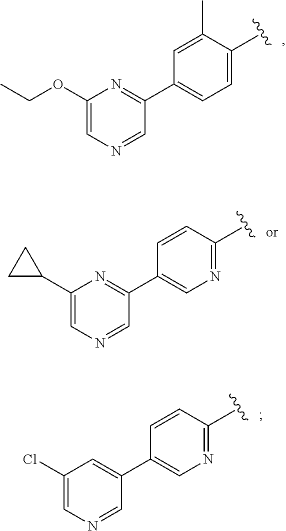

or
(f) when A, V, W, R₁, R₄, R₅, R₁₀ and R₁₂ are as follows:

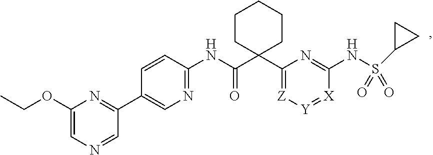

then Z, X and Y are

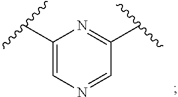 [SMILES: *c1cncc(*)n1]

or
(g) when A, V, W, R₁, R₄, R₅, R₁₀ and R₁₂ are as follows:

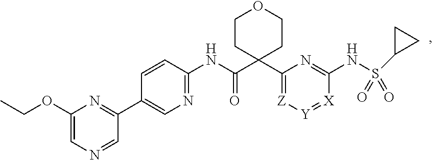

then Z, X and Y are

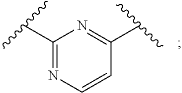 [SMILES: *c1ccnc(*)n1]

or
(h) when A, V, W, R₁, R₄, R₅, R₁₀ and R₁₂ are as follows

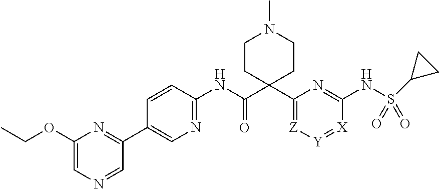

then Z, X and Y are

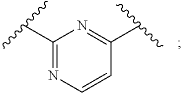 [SMILES: *c1ccnc(*)n1]

or a pharmaceutically acceptable salt and/or pharmaceutically acceptable solvate thereof.
Clause 44. The method, CTPS1 inhibitor for use, pharmaceutical composition for use or use according to clause 43, wherein the CTPS1 inhibitor is selected from the compounds disclosed in List E or a pharmaceutically acceptable salt and/or pharmaceutically acceptable solvate thereof.
Clause 45. The method, CTPS1 inhibitor for use, pharmaceutical composition for use or use according to any one of clauses 1 to 34, wherein the CTPS1 inhibitor is a compound of formula (VI):

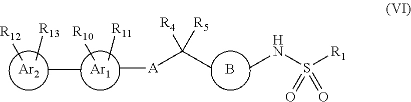

wherein ring B is selected from the group consisting of:

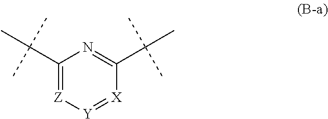

wherein X, Y and Z are as defined below; and

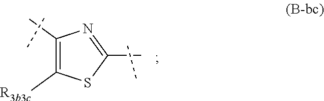

wherein R_{3b3c} is R_{3b} or R_{3c} as defined below;
wherein when B is (B-a) the compound of formula (VI) is a compound of formula (VI-a):

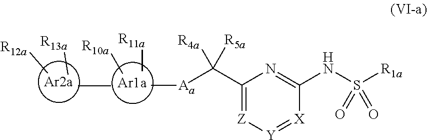

wherein:
Aₐ is Aₐₐ or A_{ba};
wherein:
   Aₐₐ is an amine linker having the following structure: —NH—, —CH₂NH—or —NHCH₂—;
   A_{ba} is an amide linker having the following structure: —C(═O)NH—
or —NHC(═O)—;
X is N or CH;
Y is N or CR₂ₐ;
Z is N or CR₃ₐ,
   with the proviso that when at least one of X or Z is N, Y cannot be N;
R₂ₐ is H, halo, C₁₋₂alkyl, OC₁₋₂alkyl, C₁₋₂haloalkyl or OC₁₋₂haloalkyl; and
R₃ₐ is H, halo, CH₃, OCH₃, CF₃ or OCF₃;
   wherein at least one of R₂ₐ and R₃ₐ is H;
R₁ₐ is R₁ₐₐ or R_{1ba};
wherein:
   R₁ₐₐ is NR₃₂ₐR₃₃ₐ;
R_{1ba} is C₁₋₅alkyl, C₀₋₂alkyleneC₃₋₅cycloalkyl which cycloalkyl is optionally substituted by CH₃, or CF₃;
R₄ₐ and R₅ₐ are R₄ₐₐ and R₅ₐₐ, or R_{4ba} and R_{5ba};
wherein:
R₄ₐₐ and R₅ₐₐ together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ; or
one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
R_{4ba} and R_{5ba} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl; and
when Aₐ is —NHC(═O)— or —NHCH₂—; R_{4ba} and R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN; R₁₁ₐ is H, F, Cl, C₁₋₂alkyl, CF₃, OCH₃ or CN;
R₁₂ₐ is attached to Ar2 in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ; and when Aₐ is —NHC(═O)—, —NH— or —NHCH₂—:
R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O—);
R₁₃ₐ is H or halo;
R₂₁ₐ is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R₂₂ₐ is H or CH₃;
R₂₃ₐ is H or C₁₋₂alkyl; and
R₂₄ₐ is H or C₁₋₂alkyl
R₂₉ₐ is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl;
R₃₂ₐ is C₁₋₃alkyl and R₃₃ is C₁₋₃alkyl; or
R₃₂ₐ and R₃₃ₐ together with the nitrogen atom to which they are attached form a C₃₋₅heterocycloalkyl;
wherein
   R₁ₐ is R₁ₐₐ; and/or
   R₄ₐ and R₅ₐ are R₄ₐₐ and R₅ₐₐ; and/or
   Aₐ is Aₐₐ; and
wherein when B is (B-bc) and R_{3b3c} is R_{3b}, the compound of formula (VI) is a compound of formula (VI-b):

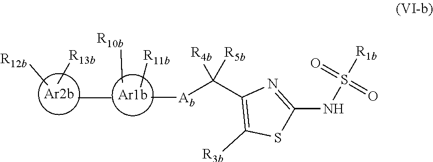

wherein:
A_{b} is A_{ab} or A_{bb};
wherein:
   A_{ab} is —NR_{6b}CH₂— or —NR_{6b}—;
   A_{bb} is —NR_{6b}C(═O)—;
R_{1b} is R_{1ab} or R_{1bb};
   wherein:
      R_{1ab} is NR_{32b}R_{33b};
R_{1bb} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃; R_{3b} is H, halo, CH₃, OC₁₋₂alkyl or CF₃;
or R_{3b} together with R_{5bb} forms a 5- or 6-membered cycloalkyl or 5 or 6 membered oxygen-containing heterocycloalkyl;
R_{4b} and R_{5b} are either R_{4ab} and R_{5ab} or R_{4bb} and R_{5bb};
wherein:
R_{4ab} and R_{5ab} together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃ haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21b}R_{22b}; or one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆cheterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
R_{4ab} and R_{5ab} together with the carbon atom to which they are attached form a C₃₋₆heterocycloalkyl comprising one nitrogen atom, wherein said nitrogen atom is substituted by —S(O)₂R_{29b}; or
R_{4bb} and R_{5bb} are each independently H, halo, C₁₋₆alkyl, C₀₋₂alkyleneC₃₋₆ cycloalkyl, C₀₋₂alkyleneC₃₋₆heterocycloalkyl, OC₁₋₆alkyl, OC₀₋₂alkyleneC₃₋₆ cycloalkyl, C₁₋₃alkyleneOC₁₋₃alkyl, C₁₋₆alkylOH, C₁₋₆haloalkyl, OC₁₋₆haloalkyl or NR_{21b}R_{22b},
or R_{4bb} is H and R_{5bb} together with R_{3b} form a 5- or 6-membered cycloalkyl or 5 or 6 membered oxygen-containing heterocycloalkyl,
or R_{4bb} and R_{5bb} together with the carbon atom to which they are attached form a C₃₋₆cycloalkyl or C₃₋₆heterocycloalkyl,
or R_{4bb} is H and R_{5bb} and R_{6b} are a C₂₋₃alkylene chain forming a 5- or 6-membered ring;
   or R_{4bb} is O and R_{5bb} is absent;
R_{6b} is H or C₁₋₃alkyl,
or R_{6b} together with R_{11b} when in the ortho-position to group A_{b} are a C₂alkylene chain forming a 5-membered ring,
or R_{5bb} and R_{6b} are a C₂₋₃alkylene chain forming a 5- or 6-membered ring and R_{4bb} is H;
Ar1 b is 6-membered aryl or heteroaryl;
Ar2b is a 6-membered aryl or heteroaryl and is attached to Ar1 b in the para position relative to group A_{b};
R_{10b} is H, halo, C₁₋₃alkyl, OC₁₋₂alkyl, C₁₋₂haloalkyl, OC₁₋₂haloalkyl or CN; R_{11b} is H, F, Cl, CH₃, ethyl, OCH₃, CF₃, OCF₃ or CN, or R_{1b}, when in the ortho-position to group A_{b}, together with R_{6b} are a C₂alkylene chain forming a 5-membered ring;
R_{12b} is attached to Ar2b in the ortho or meta position relative to Ar1 b and R_{12b} is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR_{23b}R_{24b}, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2b, or R_{12b} together with a nitrogen atom to which it is attached forms an N-oxide (N⁺—O—);
R_{13b} is H, halo, CH₃ or OCH₃; R_{21b} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22b} is H or CH₃;
R_{23b} is H or C₁₋₂alkyl;
R_{24b} is H or C₁₋₂alkyl;
R_{29b} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and R_{32b} is C₁₋₃alkyl and R_{33b} is C₁₋₃alkyl; or
R_{32b} and R_{33b} together with the nitrogen atom to which they are attached form a C₃₋₅heterocycloalkyl;
wherein:
R_{1b} is R_{1ab}; and/or
   R_{4b} and R_{5b} are R_{4ab} and R_{5ab}; and/or
   A is A_{ab}; or
wherein when B is (B-bc) and R_{3b3c} is R_{3c}, the compound of formula (VI) is a compound of formula (VI-c):

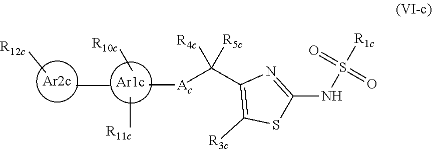

wherein:
A_{c} is A_{ac} or A_{bc};
   wherein:
A_{ac} is —CH₂NR_{6c}—;
A_{bc} is —C(═O)NR_{6c}—;
R_{1c} is R_{1ac} or R_{1bc};
   wherein:
   R_{1ac} Is NR_{32c} R_{33c};
R_{1bc} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃; R_{3c} is H, CH₃, halo, OC₁₋₂alkyl or CF₃;
R_{4c} and R_{5c} are either R_{4ac} and R_{5ac} or R_{4bc} and R_{5bc};
wherein:
R_{4ac} and R_{5ac} together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c} R_{22c}; or one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and
R_{5ac} together with the carbon atom to which they are attached may be substituted by one or two substituents, each substituent being independently selected from the group consisting of C₁₋₃alkyl or OC₁₋₃alkyl; or
R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆cheterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
R_{4ac} and R_{5ac} together with the carbon atom to which they are attached form a C₃₋₆heterocycloalkyl comprising one nitrogen atom, wherein said nitrogen atom is substituted by —S(O)₂R_{29c}; or
R_{4bc} and R_{5bc} are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl,
or R_{4bc} and R_{5bc} together with the carbon atom to which they are attached form a C₃₋₆cycloalkyl or C₃₋₆heterocycloalkyl ring; R_{6c} is H or C₁₋₃alkyl;
Ar1c is a 6-membered aryl or heteroaryl;
Ar2c is a 6-membered aryl or heteroaryl and is attached to Ar1c in the para position relative to group A_{c};
R_{10c} is H, halo, C₁₋₃alkyl, OC₁₋₂alkyl, C₁₋₂haloalkyl, OC₁₋₂haloalkyl or CN; R_{11c} is H, F, Cl, CH₃, ethyl, OCH₃, CF₃, OCF₃ or CN; R_{12c} is attached to Ar2c in the meta or ortho position relative to Ar1c and R_{12c} is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR_{23c} R_{24c}, SO₂CH₃,
C(O)N(CH₃)₂, NHC(O)C₁₋₃alkyl, or a C₃₋₆heterocycloalkyl comprising one nitrogen located at the point of attachment to Ar2c, or R_{12c} together with a nitrogen atom to which it is attached forms an N-oxide (N⁺—O—); R_{21c} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22c} is H or CH₃;
R_{23c} is H or C₁₋₂alkyl;
R_{24c} is H or C₁₋₂alkyl;
R_{29c} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32c} is C₁₋₃alkyl and R_{33c} is C₁₋₃alkyl; or
R_{32c} and R_{33c} together with the nitrogen atom to which they are attached form a C₃₋₅heterocycloalkyl;
wherein:
R_{1c} is R_{1ac}; and/or
   R_{4c} and R_{5c} are R_{4ac} and R_{5ac}; and/or
   A_{c} is A_{ac};
or a pharmaceutically acceptable salt and/or pharmaceutically acceptable solvate thereof.
Clause 46. The method, CTPS1 inhibitor for use, pharmaceutical composition for use or use according to clause 45, wherein the CTPS1 inhibitor is selected from the compounds disclosed in List F or a pharmaceutically acceptable salt and/or pharmaceutically acceptable solvate thereof.
Clause 47. The method, CTPS1 inhibitor for use, pharmaceutical composition for use or use according to any one of clauses 1 to 34, wherein the CTPS1 inhibitor is a compound of formula (VII):

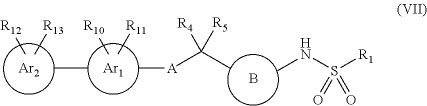

wherein
A is A, or A_{b};
wherein
   Aₐ is an amine linker having the following structure: —NH—, —
CH₂NH—or —NHCH₂—;
   A_{b} is an amide linker having the following structure: —C(═O)NH— or —NHC(═O)—; B is

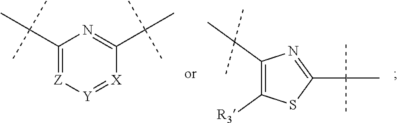

X is N or CH;
Y is N or CR₂;
Z is N or CR₃,
   with the proviso that when at least one of X or Z is N, Y cannot be N;
R₁ is C₁₋₅fluoroalkyl, with the proviso that R₁ is not CF₃;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
   wherein at least one of R₂ and R₃ is H;
R_{3′} is H, halo, CH₃, OC₁₋₂alkyl or CF₃; and
when A is —NHC(═O)—, additionally R_{3′} together with R₅ forms a 5- or 6-membered cycloalkyl or 5 or 6 membered oxygen-containing heterocycloalkyl;
R₄ and R₅ are R₄ₐ and R₅ₐ, or R_{4b} and R_{5b};
wherein
R₄ₐ and R₅ₐ together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁R₂₂; or one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐ and
R₅ₐ together with the carbon atom to which they are attached may be substituted by one or two substituents, each substituent being independently selected from the group consisting of C₁₋₃alkyl or OC₁₋₃alkyl; or R₄ₐ and R₅ₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
R₄ₐ and R₅ₐ together with the carbon atom to which they are attached form a C₃₋₆heterocycloalkyl comprising one nitrogen atom, wherein said nitrogen atom is substituted by —S(O)₂R₂₉; or
R_{4b} and R_{5b} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4b} and R_{5b} together with the carbon atom to which they are attached form a C₃₋₆cycloalkyl or C₃₋₆heterocycloalkyl; and
when A is —NHC(═O)— or —NHCH₂—;
R_{4b} and R_{5b} may additionally be selected from halo, OCl₆haloalkyl, OC₀₋₂alkyleneC₃₋₆cycloalkyl, OC₀₋₂alkyleneC₃₋₆heterocycloalkyl, OCl₆alkyl and NR₂₁R₂₂;
Ar1 is a 6-membered aryl or heteroaryl;
Ar2 is a 6-membered aryl or heteroaryl and is attached to Ar1 in the para position relative to group A;
R₁₀ is H, halo, C₁₋₃alkyl, C₁₋₂haloalkyl, OC₁₋₂alkyl, OC₁₋₂haloalkyl or CN;
Ru is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
   when A is —NHC(═O)—, —NH— or —NHCH₂—:
R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O—);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
R₂₉ is C₁₋₃alkyl, C₀₋₂alkyleneC₃₋₅cycloalkyl which cycloalkyl is optionally substituted by CH₃, or CF₃;
R₃₂ is C₁₋₃alkyl and R₃₃ is C₁₋₃alkyl; or
R₃₂ and R₃₃ together with the nitrogen atom to which they are attached form a C₃₋₅heterocycloalkyl;
or a pharmaceutically acceptable salt and/or pharmaceutically acceptable solvate thereof.
Clause 48. The method, CTPS1 inhibitor for use, pharmaceutical composition for use or use according to clause 47, wherein the CTPS1 inhibitor is selected from the compounds disclosed in List G or a pharmaceutically acceptable salt and/or pharmaceutically acceptable solvate thereof.
Clause 49. The method, CTPS1 inhibitor for use, pharmaceutical composition for use or use according to any one of clauses 1 to 34, wherein the CTPS1 inhibitor is compound of formula (VIII):

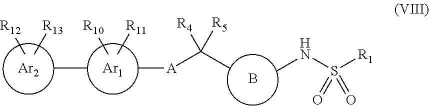

wherein
A is A, or A_{b};
wherein
   Aₐ is an amine linker having the following structure: —NH—, —
CH₂NH—or —NHCH₂—;
   A_{b} is an amide linker having the following structure: —C(═O)NH— or —NHC(═O)—;
B is

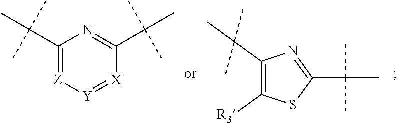

X is N or CH;
Y is N or CR₂;
Z is N or CR₃,
   with the proviso that when at least one of X or Z is N, Y cannot be N;
R₁ is C₁₋₅alkyl or C₀₋₂alkyleneC₃₋₅cycloalkyl, which alkyl or (alkylene)cycloalkyl is substituted by CN;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
   wherein at least one of R₂ and R₃ is H;
R₃, is H, halo, CH₃, OC₁₋₂alkyl or CF₃; and
when A is —NHC(═O)—, additionally R₃, together with R₅ forms a 5- or 6-membered cycloalkyl or 5 or 6 membered oxygen-containing heterocycloalkyl; R₄ and R₅ are R₄ₐ and R₅ₐ, or R_{4b} and R_{5b};
wherein
R₄ₐ and R₅ₐ together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁R₂₂; or one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐ and
R₅ₐ together with the carbon atom to which they are attached may be substituted by one or two substituents, each substituent being independently selected from the group consisting of C₁₋₃alkyl or OC₁₋₃alkyl; or
R₄ₐ and R₅ₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐ and R₅ₐ together with the carbon atom to which they are attached may be substituted by one or two substituents, each substituent being independently selected from the group consisting of C₁₋₃alkyl or OC₁₋₃alkyl; or
R₄ₐ and R₅ₐ together with the carbon atom to which they are attached form a C₃₋₆heterocycloalkyl comprising one nitrogen atom, wherein said nitrogen atom is substituted by —S(O)₂R₂₉; or
R_{4b} and R_{5b} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4b} and R_{5b} together with the carbon atom to which they are attached form a C₃₋₆cycloalkyl or C₃₋₆heterocycloalkyl; and
when A is —NHC(═O)— or —NHCH₂—;
R_{4b} and R_{5b} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to group A;
R₁₀ is H, halo, C₁₋₃alkyl, C₁₋₂haloalkyl, OC₁₋₂alkyl, OC₁₋₂haloalkyl or CN; R₁₁ is H, F, Cl, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OCl₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and when A is —NHC(═O)—, —NH— or —NHCH₂—:
R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O—);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
R₂₉ is C₁₋₃alkyl, C₀₋₂alkyleneC₃₋₅cycloalkyl which cycloalkyl is optionally substituted by CH₃, or CF₃;
R₃₂ is C₁₋₃alkyl and R₃₃ is C₁₋₃alkyl; or
R₃₂ and R₃₃ together with the nitrogen atom to which they are attached form a C₃₋₅heterocycloalkyl;
or a pharmaceutically acceptable salt and/or pharmaceutically acceptable solvate thereof.
Clause 50. The method, CTPS1 inhibitor for use, pharmaceutical composition for use or use according to clause 49, wherein the CTPS1 inhibitor is selected from the compounds disclosed in List H or a pharmaceutically acceptable salt and/or pharmaceutically acceptable solvate thereof.
Clause 51. The method, CTPS1 inhibitor for use, pharmaceutical composition for use or use according to any one of clauses 1 to 34, wherein the CTPS1 inhibitor is 4-(2-(cyclopropanesulfonamido)pyrimidin-4-yl)-N-(5-(6-ethoxypyrazin-2-yl)pyridin-2-yl)tetrahydro-2H-pyran-4-carboxamide:

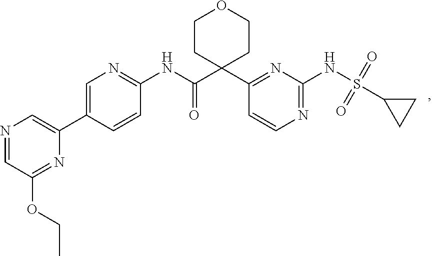 [SMILES: CCOc1cncc(-c2ccc(NC(=O)C3(c4ccnc(NS(=O)(=O)C5CC5)n4)CCOCC3)nc2)n1]

or a pharmaceutically acceptable salt and/or pharmaceutically acceptable solvate thereof.
Clause 52. The method, CTPS1 inhibitor for use, pharmaceutical composition for use or use according to any one of clauses 1 to 31, wherein the CTPS1 inhibitor is N-(5-(6-ethoxypyrazin-2-yl)pyridin-2-yl)-4-(2-(ethylsulfonamido)pyrimidin-4-yl)tetrahydro-2H-pyran-4-carboxamide:

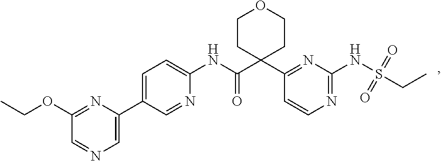 [SMILES: CCOc1cncc(-c2ccc(NC(=O)C3(c4ccnc(NS(=O)(=O)CC)n4)CCOCC3)nc2)n1]

or a pharmaceutically acceptable salt and/or pharmaceutically acceptable solvate thereof.
Clause 53. The method, CTPS1 inhibitor for use, pharmaceutical composition for use or use according to any one of clauses 1 to 52, wherein the CTPS1 inhibitor is in its free form.
Clause 54. The method, CTPS1 inhibitor for use, pharmaceutical composition for use or use according to any one of clauses 1 to 52, wherein the CTPS1 inhibitor is a pharmaceutically acceptable salt.
Clause 55. The method, CTPS1 inhibitor for use, pharmaceutical composition for use or use according to any one of clauses 1 to 52, wherein the CTPS1 inhibitor is a pharmaceutically acceptable solvate.
Clause 56. The method, CTPS1 inhibitor for use, pharmaceutical composition for use or use according to any one of clauses 1 to 52, wherein the CTPS1 inhibitor is a pharmaceutically acceptable salt and a pharmaceutically acceptable solvate.
Clause 57. The method, CTPS1 inhibitor for use, pharmaceutical composition for use or use according to any one of clauses 1 to 34, wherein the CTPS1 inhibitor is 4-(2-(cyclopropanesulfonamido)pyrimidin-4-yl)-N-(5-(6-ethoxypyrazin-2-yl)pyridin-2-yl)tetrahydro-2H-pyran-4-carboxamide (‘CTPS1-IA’):

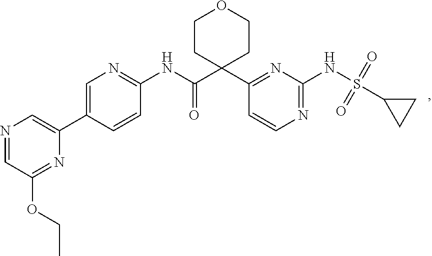 [SMILES: CCOc1cncc(-c2ccc(NC(=O)C3(c4ccnc(NS(=O)(=O)C5CC5)n4)CCOCC3)nc2)n1]

or a pharmaceutically acceptable salt thereof.
Clause 58. The method, CTPS1 inhibitor for use, pharmaceutical composition for use or use according to any one of clauses 1 to 34, wherein the CTPS1 inhibitor is N-(5-(6-ethoxypyrazin-2-yl)pyridin-2-yl)-4-(2-(ethylsulfonamido)pyrimidin-4-yl)tetrahydro-2H-pyran-4-carboxamide (‘CTPS1-IB’):

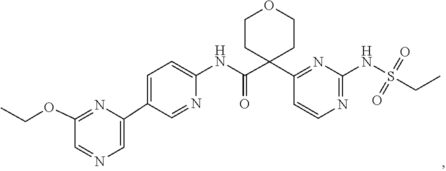 [SMILES: CCOc1cncc(-c2ccc(NC(=O)C3(c4ccnc(NS(=O)(=O)CC)n4)CCOCC3)nc2)n1]

or a pharmaceutically acceptable salt thereof.
Clause 59. The method, CTPS1 inhibitor for use, pharmaceutical composition for use or use according to any one of clauses 1 to 58, wherein the CTPS1 inhibitor is provided in a natural isotopic form.
Clause 60. The method, CTPS1 inhibitor for use, pharmaceutical composition for use or use according to any one of clauses 1 to 59, wherein the CTPS1 inhibitor is a CTPS1 inhibitor as defined in claim 1 of WO2022/087634.
Clause 61. The method, CTPS1 inhibitor for use, pharmaceutical composition for use or use according to any one of clauses 1 to 59, wherein the CTPS1 inhibitor is a CTPS1 inhibitor as defined in WO2022/087634.
Clause 62. The method, CTPS1 inhibitor for use, pharmaceutical composition for use or use according to any one of clauses 1 to 59, wherein the CTPS1 inhibitor is not a CTPS1 inhibitor as defined in claim 1 of WO2022/087634.
Clause 63. The method, CTPS1 inhibitor for use, pharmaceutical composition for use or use according to any one of clauses 1 to 59, wherein the CTPS1 inhibitor is not a CTPS1 inhibitor as defined in WO2022/087634.
Clause 64. The method, CTPS1 inhibitor for use, pharmaceutical composition for use or use according to any one of clauses 1 to 63, wherein deficiency in CTPS2 results from a genomic alteration or alterations and/or an epigenic change or changes.
Clause 65. The method, CTPS1 inhibitor for use, pharmaceutical composition for use or use according to any one of clauses 1 to 64, wherein deficiency in CTPS2 results from a genomic alteration or alterations.
Clause 66. The method, CTPS1 inhibitor for use, pharmaceutical composition for use or use according to clause 64 or 65, wherein the genomic alteration or alterations are complete loss of the CTPS2 gene by genomic deletion.
Clause 67. The method, CTPS1 inhibitor for use, pharmaceutical composition for use or use according to any one of clauses 64 to 66, wherein the genomic alteration or alterations are partial loss of the CTPS2 gene by genomic deletion.
Clause 68. The method, CTPS1 inhibitor for use, pharmaceutical composition for use or use according to any one of clauses 64 to 67, wherein the genomic alteration or alterations are disruption of the CTPS2 gene by a structural DNA variant such as an inversion, duplication or translocation falling within the footprint of the gene.
Clause 69. The method, CTPS1 inhibitor for use, pharmaceutical composition for use or use according to any one of clauses 64 to 68, wherein the genomic alteration or alterations are mutation of the CTPS2 gene such that CTPS2 expression is substantially reduced, or most suitably completely lost.
Clause 70. The method, CTPS1 inhibitor for use, pharmaceutical composition for use or use according to any one of clauses 64 to 68, wherein deficiency in CTPS2 results from an epigenic change or changes.
Clause 71. The method, CTPS1 inhibitor for use, pharmaceutical composition for use or use according to clause 70, wherein the epigenic change or changes are altered expression of the CTPS2 gene, for example due to changes in regulatory elements due to altered methylation and/or histone modification, such that CTPS2 expression is substantially reduced, or most suitably completely lost.
Clause 72. The method, CTPS1 inhibitor for use, pharmaceutical composition for use or use according to any one of clauses 1 to 71, wherein the sample is a biopsy containing cancer cells (e.g. a tumour biopsy), a sample containing circulating cancer cells or a sample containing cell free cancer DNA.
Clause 73. The method, CTPS1 inhibitor for use, pharmaceutical composition for use or use according to any one of clauses 1 to 72, wherein the step identifying that a subject has a cancer which is deficient in CTPS2 comprises:
   (i) applying a suitable method for determining CTPS2 deficiency, such as a technique to detect relevant alteration of DNA, RNA or protein,
   (ii) analysis of data produced by the method, and
   (iii) interpretation of the data produced by the method to determine the likelihood of a substantial deficiency in CTPS2 function, in particular a complete loss of CTPS2 function.
Clause 74. The method, CTPS1 inhibitor for use, pharmaceutical composition for use or use according to clause 73, wherein the method for determining CTPS2 deficiency is a method which detects DNA changes e.g. whole genome sequencing, whole exome sequencing, targeted gene sequencing using capture based enrichment, targeted gene sequencing using PCR-based enrichment, real-time quantitative PCR, digital droplet PCR, in situ hybridization or fluorescence in situ hybridization.
Clause 75. The method, CTPS1 inhibitor for use, pharmaceutical composition for use or use according to clause 73 or 74, wherein the method for determining CTPS2 deficiency is a method which detects RNA changes e.g. RNA sequencing, gene expression array, real-time quantitative PCR, digital droplet PCR or in situ hybridization.
Clause 76. The method, CTPS1 inhibitor for use, pharmaceutical composition for use or use according to any one of clauses 73 to 75, wherein the method for determining CTPS2 deficiency is a method which detects epigenic changes e.g. DNA methylation analysis, characterisation of histone modification, RNA sequencing, gene expression array, real-time quantitative PCR, digital droplet PCR or in situ hybridization.
Clause 77. The method, CTPS1 inhibitor for use, pharmaceutical composition for use or use according to any one of clauses 73 to 76, wherein the method for determining CTPS2 deficiency is a method which detects protein changes e.g. immunohistochemistry, flow cytometry or mass cytometry.
Clause 78. The method, CTPS1 inhibitor for use, pharmaceutical composition for use or use according to clauses 77, wherein the method for determining CTPS2 deficiency is immunohistochemistry, such as absence of staining for CTPS2.
Clause 79. The method, CTPS1 inhibitor for use, pharmaceutical composition for use or use according to any one of clauses 1 to 78, wherein the subject is a human subject.
Clause 80. The method, CTPS1 inhibitor for use, pharmaceutical composition for use or use according to clause 79, wherein the human subject is adult, such as aged 18 to 65.
Clause 81. The method, CTPS1 inhibitor for use, pharmaceutical composition for use or use according to clause 79, wherein the human subject is less than 18 years of age, such as 4 to 17 years old.
Clause 82. The method, CTPS1 inhibitor for use, pharmaceutical composition for use or use according to clause 79, wherein the human subject is 66 years or older.
Clause 83. The method, CTPS1 inhibitor for use, pharmaceutical composition for use or use according to any one of clauses 79 to 82, wherein the human subject is male.
Clause 84. The method, CTPS1 inhibitor for use, pharmaceutical composition for use or use according to clause 83, wherein deficiency in CTPS2 results from the complete loss of the CTPS2 gene by genomic deletion.
Clause 85. The method, CTPS1 inhibitor for use, pharmaceutical composition for use or use according to any one of clauses 79 to 82, wherein the human subject is female.
Clause 86. The method, CTPS1 inhibitor for use, pharmaceutical composition for use or use according to clause 85, wherein CTPS2 deficiency results from genomic alteration(s) and/or epigenic change(s) in one or both XX chromosomes, suitably both XX chromosomes.
Clause 87. The method, CTPS1 inhibitor for use, pharmaceutical composition for use or use according to clause 85 or 86, wherein deficiency in CTPS2 results from complete loss of the CTPS2 gene on a first X chromosome by genomic deletion, and:
   (i) complete loss of the CTPS2 gene on a second X chromosome by genomic deletion; (ii) partial loss of the CTPS2 gene on a second X chromosome by genomic deletion; (iii) disruption of the CTPS2 gene on a second X chromosome by a structural DNA variant such as an inversion, duplication or translocation falling within the footprint of the gene;
   (iv) mutation of the CTPS2 gene on a second X chromosome such that CTPS2 expression is substantially reduced, or completely lost; or (v) altered expression of the CTPS2 gene on a second X chromosome, for example due to changes in regulatory elements due to altered methylation and/or histone modification, such that CTPS2 expression is substantially reduced, or completely lost.
Clause 88. The method, CTPS1 inhibitor for use, pharmaceutical composition for use or use according to clause 86 or 87, wherein deficiency in CTPS2 results from complete loss of the CTPS2 gene on a first X chromosome by genomic deletion and complete loss of the CTPS2 gene on a second X chromosome by genomic deletion.
Clause 89. The method, CTPS1 inhibitor for use, pharmaceutical composition for use or use according to clause any one of clauses 1 to 88, wherein deficiency in CTPS2 results from homozygous deletion in cancer cells.
Clause 90. The method, CTPS1 inhibitor for use, pharmaceutical composition for use or use according to any one of clauses 1 to 89, wherein the CTPS1 inhibitor is administered orally.
Clause 91. The method, CTPS1 inhibitor for use, pharmaceutical composition for use or use according to clause 90, wherein the CTPS1 inhibitor is administered in a solid pharmaceutical composition (such as a tablet, capsule or lozenge).
Clause 92. The method, CTPS1 inhibitor for use, pharmaceutical composition for use or use according to clause 90, wherein the CTPS1 inhibitor is administered in a liquid pharmaceutical composition (such as a suspension, emulsion or solution).
Clause 93. The method, CTPS1 inhibitor for use, pharmaceutical composition for use or use according to any one of clauses 1 to 92, wherein the CTPS1 inhibitor is administered at a unit dose of 0.05 mg to 1000 mg, more suitably 1.0 mg to 500 mg.
Clause 94. The method, CTPS1 inhibitor for use, pharmaceutical composition for use or use according to any one of clauses 1 to 93, wherein the CTPS1 inhibitor is administered more than once or twice a day e.g. two or three times a day.
Clause 95. The method, CTPS1 inhibitor for use, pharmaceutical composition for use or use according to any one of clauses 1 to 94, wherein the CTPS1 inhibitor is administered continuously e.g. at least daily for a period of multiple weeks.
Clause 96. The method, CTPS1 inhibitor for use, pharmaceutical composition for use or use according to any one of clauses 1 to 94, wherein the CTPS1 inhibitor is administered discontinuously e.g. at least daily for one week, followed by a week without administration, followed at least daily for one further week.
Clause 97. The method, CTPS1 inhibitor for use, pharmaceutical composition for use, pharmaceutical composition for use or use according to any one of clauses 1 to 96, wherein the CTPS1 inhibitor is administered in combination with one or more further pharmaceutically acceptable active ingredients selected from the group consisting of: anti-mitotic agents such as vinblastine, paclitaxel and docetaxel; alkylating and DNA-cross linking agents, for example cisplatin, carboplatin, dacarbazine and cyclophosphamide; antimetabolites, for example 5-fluorouracil, cytosine arabinoside and hydroxyurea; intercalating agents for example adriamycin and bleomycin; topoisomerase inhibitors for example etoposide, topotecan and irinotecan; thymidylate synthase inhibitors for example raltitrexed; P13-kinase inhibitors for example idelalisib; MTOR inhibitors for example everolimus and temsirolimus; proteasome inhibitors for example bortezomib; histone deacetylase inhibitors for example panobinostat or vorinostat; hedgehog pathway blockers such as vismodegib; IAP inhibitors such as LCL161; VEGF inhibitors such as bevacizumab; pan-kinase inhibitors such as sorafenib and sunitinib; tyrosine kinase inhibitors such as, for example, axitinib, dasatinib, erlotinib, imatinib, nilotinib, pazopanib and sunitinib; azacitidine, decitabine, and cytarabine.
Clause 98. The method, CTPS1 inhibitor for use, pharmaceutical composition for use, pharmaceutical composition for use or use according to any one of clauses 1 to 97, wherein the CTPS1 inhibitor is administered in combination with one or more anticancer antibodies, such as (such as obinutuzumab, ofatumumab, tositumomab or rituximab), olaratumab, daratumumab, necitumumab, dinutuximab, traztuzumab emtansine, pertuzumab, brentuximab, panitumumab, catumaxomab, bevacizumab, cetuximab, traztuzumab and gentuzumab ozogamycin.
Clause 99. The method, CTPS1 inhibitor for use, pharmaceutical composition for use, pharmaceutical composition for use or use according to any one of clauses 1 to 98, wherein the CTPS1 inhibitor is administered in combination with radiotherapy, surgery, hyperthermia therapy and/or cryotherapy.
Clause 100. The method, CTPS1 inhibitor for use, pharmaceutical composition for use, pharmaceutical composition for use or use according to any one of clauses 1 to 99, wherein the subject received chemotherapy, radiotherapy, surgery, hyperthermia therapy and/or cryotherapy prior to CTPS1 administration.
Clause 101. The method, CTPS1 inhibitor for use, pharmaceutical composition for use, pharmaceutical composition for use or use according to any one of clauses 1 to 100, wherein the subject receives chemotherapy, radiotherapy, surgery, hyperthermia therapy and/or cryotherapy concurrently with CTPS1 administration.
Clause 102. The method, CTPS1 inhibitor for use, pharmaceutical composition for use, pharmaceutical composition for use or use according to any one of clauses 1 to 101, wherein the subject receives chemotherapy, radiotherapy, surgery, hyperthermia therapy and/or cryotherapy after to CTPS1 administration.
Clause 103. The method, CTPS1 inhibitor for use, pharmaceutical composition for use, pharmaceutical composition for use or use according to any one of clauses 1 to 102, wherein the cancer or cancer cells are from a cancer type in which at least 10% are deficient in CTPS2, such as at least 15% of are deficient in CTPS2, especially as at least 18% are deficient in CTPS2.
Clause 104. The method, CTPS1 inhibitor for use, pharmaceutical composition for use, pharmaceutical composition for use or use according to clause 103, wherein deficiency in CTPS2 function is due to homozygous deletion arising in females or hemizygous deletion arising in males.
Clause 105. The method, CTPS1 inhibitor for use, pharmaceutical composition for use, pharmaceutical composition for use or use according to any one of clauses 1 to 104, wherein the cancer or cancer cells are from a cancer type in which at least 10% are deficient in CTPS2, such as at least 15% of are deficient in CTPS2, especially as at least 20% are deficient in CTPS2, in particular in particular at least 40% are deficient in CTPS2.
Clause 106. The method, CTPS1 inhibitor for use, pharmaceutical composition for use, pharmaceutical composition for use or use according to clause 105, wherein deficiency in CTPS2 function is be determined by immunohistochemistry, such as absence of staining for CTPS2.

### REFERENCES

Anderson et al. Blood. 2016 Jun. 23; 127(25):3215-3224.
Behan et al. Nature. 2019 April; 568(7753):511-516.
B/biogps.org/, EMBL-EBI Expression s://www.ebi.ac.uk/gxa/home
Bliss Ann. Appl. Biol. 1939 26, 585-615.
Cancer Dependenc/depmap.org/
Casara et al. Oncotarget. 2018 Apr. 13; 9(28):20075-20088.
CellMineiscover.nci.nih.gov/rsconnect/cellminercdb/
Diepstraten et al. Nat Rev Cancer. 2022 January;22(1):45-64.
Doepner et al. Nature. 2019 April; 568(7753):511-516.
EMBL-EBI Expression s://www.ebi.ac.uk/gxa/home
Li et al. Cancer Chemother Pharmacol. 2008 March;61(3):525-34.
Martin et al. Nature. 2014 Jun. 12; 510(7504):288-292.
Martin et al. JCI Insight. 2020 Mar. 12; 5(5):e133880.
Minet et al. Life Science Alliance 2023 6(9):e202302066 DOI 10.26508/lsa.202302066
Park et al. J Med Chem. 2008 Nov. 13; 51(21):6902-6915.
Punnoose et al. Mol Cancer Ther. 2016 May; 15(5):1132-1144.
Roberts et al. Blood. 2021 Sep. 30; 138(13):1120-1136.
Souers et al. Nat Med. 2013 February;19(2):202-208.
Tse et al. Cancer Res. 2008 May 1;68(9):3421-3428.
Tutusaus et al. Oncotarget. 2018 Mar. 30; 9(24):16701-16717.
van Kuilenburg et al. Biochim Biophys Acta. 2000 Jul. 24; 1492(2-3):548-552.
Wang et al. J Med Chem. 2006 Oct. 19; 49(21):6139-6142.
Warren et al. Cell Death Dis. 2019 Feb. 21; 10(3):177.
Zhang et al. Anal Biochem. 2002 Aug. 1; 307(1):70-75.
Zheng et al. bioRxiv, 2021.06.01.446564.
WO2019106146
WO2019106156
WO2019179652
WO2019180244
WO2020083975
WO2020245664
WO2020245665
WO2021053402
WO2021053403
WO2022087634
Minet et al. Differential roles of CTP1 and CTPS2 in cell proliferation, submitted for publication. Lynch E M, DiMattia M A, Albanese S, van Zundert G C P, Hansen J M, Quispe J D, Kennedy M A, Verras A, Borrelli K, Toms A V, Kaila N, Kreutter K D, McElwee J J, Kollman J M. Structural basis for isoform-specific inhibition of human CTPS1. Proc Natl Acad Sci USA. 2021 Oct. 5; 118(40):e2107968118.
ICGC/TCGA Pan-Cancer Analysis of Whole Genomes Consortium. Pan-cancer analysis of whole genomes. Nature. 2020 February;578(7793):82-93.
Achreja A, Yu T, Mittal A, Choppara S, Animasahun 0, Nenwani M, Wuchu F, Meurs N, Mohan A, Jeon J H, Sarangi I, Jayaraman A, Owen S, Kulkarni R, Cusato M, Weinberg F, Kweon H K, Subramanian C, Wicha M S, Merajver S D, Nagrath S, Cho K R, DiFeo A, Lu X, Nagrath D. Metabolic collateral lethal target identification reveals MTHFD2 paralogue dependency in ovarian cancer. Nat Metab. 2022 September;4(9):1119-1137.

What is claimed is:

1-31. (canceled)

32. A method for the treatment of CTPS2 deficient cancer in a subject, the method comprising administering a CTPS1 inhibitor to the subject.

33. A method for the treatment of cancer in a subject, the method comprising the steps of:
i) identifying that the subject has a cancer which is deficient in CTPS2; and
ii) administering a CTPS1 inhibitor to the subject.

34. A method for the treatment of cancer which may be susceptible to treatment with a CTPS1 inhibitor in a subject, the method comprising the steps of:
i) identifying that the cancer is deficient in CTPS2; and
ii) administering a CTPS1 inhibitor.

35. A method of determining that cancer cells may be susceptible to treatment with a CTPS1 inhibitor, the method comprising the step of identifying that the cancer cells are deficient in CTPS2.

36. A method of determining that a cancer in a subject may be susceptible to treatment with a CTPS1 inhibitor, the method comprising the step of identifying that the cancer is deficient in CTPS2.

37. The method according to claim 32, wherein the CTPS2 deficient cancer is selected from ovarian cancer, oesophageal cancer, bladder cancer, non-small cell lung cancer, gastric cancer, sarcoma, head and neck cancer, pancreatic adeno cancer, pancreatic neuroendocrine tumours, biliary tract cancer, melanoma, endometrial cancer, hepatocellular cancer, cervical cancer, bone cancer, central nervous system cancer, breast cancer, prostate cancer, colorectal cancer and renal cancer.

38. The method according to claim 37, wherein the cancer is ovarian cancer, such as serous ovarian cancer.

39. The method according to claim 37, wherein the cancer is lung cancer, such as non-small cell lung cancer.

40. The method according to claim 37, wherein the cancer is oesophageal cancer.

41. The method according to claim 37, wherein the cancer is gastric cancer.

42. The method according to claim 37, wherein the cancer is head and neck cancer.

43. The method according to claim 37, wherein the cancer is bladder cancer.

44. The method according to claim 37, wherein the cancer is sarcoma.

45. The method according to claim 32, wherein the CTPS1 inhibitor is a compound of formula (III): 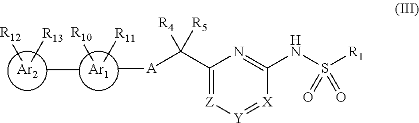
wherein
A is an amide linker having the following structure: —C(═O)NH— or —NHC(═O)—;
X is N or CH;
Y is N or CR₂;
Z is N or CR₃,
with the proviso that when at least one of X or Z is N, Y cannot be N;
R₁ is C₁₋₅alkyl, C₀₋₂alkyleneC₃₋₅cycloalkyl which cycloalkyl is optionally substituted by CH₃, or CF₃;
R₂ is H, halo, C₁₋₂alkyl, OC₁₋₂alkyl, C₁₋₂haloalkyl or OC₁₋₂haloalkyl;
R₃ is H, halo, CH₃, OCH₃, CF₃ or OCF₃;
wherein at least one of R₂ and R₃ is H;
R₄ and R₅ are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R₄ and R₅ together with the carbon atom to which they are attached form a C₃₋₆cycloalkyl or C₃₋₆-heterocycloalkyl; and
when A is —NHC(═O)—:
R₄ and R₅ may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁R₂₂;
Ar1 is a 6-membered aryl or heteroaryl;
Ar2 is a 6-membered aryl or heteroaryl and is attached to Ar1 in the para position relative to the amide;
R₁₀ is H, halo, C₁₋₃alkyl, C₁₋₂haloalkyl, OC₁₋₂alkyl, OC₁₋₂haloalkyl or CN;
R₁₁ is H, F, C, C₁₋₂alkyl, CF₃, OCH₃ or CN;
R₁₂ is attached to Ar2 in the ortho or meta position relative to Ar1 and R₁₂ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃R₂₄; and
when A is —NHC(═O)—:
R₁₂ may additionally be selected from CN, OCH₂CH₂N(CH₃)₂ and a C₃₋₆heterocycloalkyl comprising one nitrogen located at the point of attachment to Ar2, or R₁₂ together with a nitrogen atom to which it is attached forms an N-oxide (N⁺—O—);
R₁₃ is H or halo;
R₂₁ is H, C₁₋₅alkyl, C(O)C₁₋₅alkyl, C(O)OC₁₋₅alkyl;
R₂₂ is H or CH₃;
R₂₃ is H or C₁₋₂alkyl; and
R₂₄ is H or C₁₋₂alkyl;
or a pharmaceutically acceptable salt and/or pharmaceutically acceptable solvate thereof.

46. The method according to claim 45, wherein the CTPS1 inhibitor is 4-(2-(cyclopropanesulfonamido)pyrimidin-4-yl)-N-(5-(6-ethoxypyrazin-2-yl)pyridin-2-yl)tetrahydro-2H-pyran-4-carboxamide: 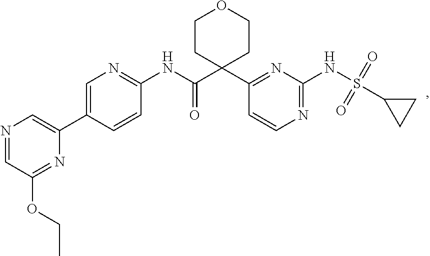 [SMILES: CCOc1cncc(-c2ccc(NC(=O)C3(c4ccnc(NS(=O)(=O)C5CC5)n4)CCOCC3)nc2)n1]
or a pharmaceutically acceptable salt and/or pharmaceutically acceptable solvate thereof.

47. The method according to claim 32, wherein the deficiency in CTPS2 results from a genomic alteration or alterations and/or an epigenic change or changes.

48. The method according to claim 47, wherein the genomic alteration or alterations are complete loss of the CTPS2 gene by genomic deletion.

49. The method according to claim 32, wherein the subject is a human male.

50. The method according to claim 32, wherein the subject is a human female.

51. The method according to claim 32, wherein the deficiency in CTPS2 results from homozygous deletion in cancer cells.

52. The method according to claim 37, wherein the deficiency in CTPS2 results from homozygous deletion in cancer cells.

53. The method according to claim 32, wherein the deficiency in CTPS2 is determined by methods which detect DNA changes.

54. The method according to claim 32, wherein the deficiency in CTPS2 is determined by methods which detect RNA changes.

55. The method according to claim 32, wherein the deficiency in CTPS2 is determined by methods which detect epigenetic changes.

56. The method according to claim 32, wherein the deficiency in CTPS2 is determined by methods which detect protein changes, such as immunohistochemistry.

57. The method according to claim 32, wherein the CTPS1 inhibitor is 4-(2-(cyclopropanesulfonamido)pyrimidin-4-yl)-N-(5-(6-ethoxypyrazin-2-yl)pyridin-2-yl)tetrahydro-2H-pyran-4-carboxamide: 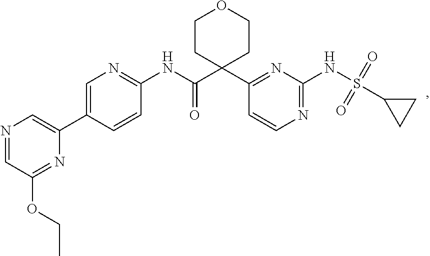 [SMILES: CCOc1cncc(-c2ccc(NC(=O)C3(c4ccnc(NS(=O)(=O)C5CC5)n4)CCOCC3)nc2)n1]
or a pharmaceutically acceptable salt and/or pharmaceutically acceptable solvate thereof.

58. The method according to claim 52, wherein the CTPS1 inhibitor is 4-(2-(cyclopropanesulfonamido)pyrimidin-4-yl)-N-(5-(6-ethoxypyrazin-2-yl)pyridin-2-yl)tetrahydro-2H-pyran-4-carboxamide: 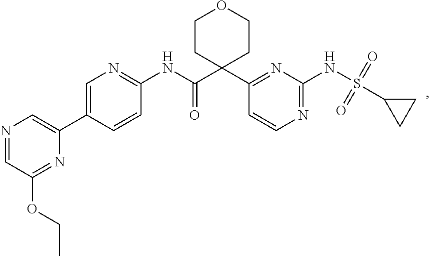 [SMILES: CCOc1cncc(-c2ccc(NC(=O)C3(c4ccnc(NS(=O)(=O)C5CC5)n4)CCOCC3)nc2)n1]
or a pharmaceutically acceptable salt and/or pharmaceutically acceptable solvate thereof.


SEQUENCE LISTING (2 sequences)

SEQ ID NO: 1 - protein, 20 aa, synthetic construct
GGDYKDDDDKGGHHHHHHHH

SEQ ID NO: 2 - protein, 29 aa, synthetic construct
DYKDDDDKHHHHHHGLNDIFEAQKIEWHE